SEQUENCE LISTING (167 sequences)

SEQ ID NO: 1 - protein, 8 aa, synthetic construct
  REGION 1..8: note = Based on llima sequence
GSTWSINT

SEQ ID NO: 2 - protein, 7 aa, synthetic construct
  REGION 1..7: note = Based on llima sequence
ISSGGST

SEQ ID NO: 3 - protein, 10 aa, synthetic construct
  REGION 1..10: note = Based on llima sequence
YAQSTWYPPS

SEQ ID NO: 4 - protein, 25 aa, Homo sapiens [sequence omitted]

SEQ ID NO: 5 - protein, 17 aa, Homo sapiens
LAWYRQAPGKQRDLVAR

SEQ ID NO: 6 - protein, 38 aa, Homo sapiens
YYADSVKGRFTISRDNSKNTLYLQMNSLRAEDTAVYYC

SEQ ID NO: 7 - protein, 11 aa, Homo sapiens
WGQGTLVTVSS

SEQ ID NO: 8 - protein, 116 aa, synthetic construct
  REGION 1..116: note = Based on llima sequence
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSS

SEQ ID NO: 9 - protein, 8 aa, synthetic construct
  REGION 1..8: note = Based on llima sequence
GFAFRGFG

SEQ ID NO: 10 - protein, 8 aa, synthetic construct
  REGION 1..8: note = Based on llima sequence
INNGGSDT

SEQ ID NO: 11 - protein, 9 aa, synthetic construct
  REGION 1..9: note = Based on llima sequence
AIGGPGASP

SEQ ID NO: 12 - protein, 25 aa, Homo sapiens
QVQLVESGGGVVQPGGSLRLSCAAS

SEQ ID NO: 13 - protein, 17 aa, Homo sapiens
MSWVRQAPGKGLEWVSS

SEQ ID NO: 14 - protein, 11 aa, Homo sapiens
SGQGTQVTVSS

SEQ ID NO: 15 - protein, 116 aa, synthetic construct
  REGION 1..116: note = Based on llima sequence
QVQLVESGGGVVQPGGSLRLSCAASGFAFRGFGMSWVRQAPGKGLEWVSSINNGGSDTYYADSVKGRFTISRDNSKNTLYLQMNSLRAEDTAVYYCAIGGPGASPSGQGTQVTVSS

SEQ ID NO: 16 - protein, 6 aa, synthetic construct
  REGION 1..6: note = Linker
GSGGSG

SEQ ID NO: 17 - protein, 9 aa, synthetic construct
  REGION 1..9: note = Linker
GSGGSGGSG

SEQ ID NO: 18 - protein, 12 aa, synthetic construct
  REGION 1..12: note = Linker
GSGGSGGSGGSG

SEQ ID NO: 19 - protein, 4 aa, synthetic construct
  REGION 1..4: note = Linker
GGGS

SEQ ID NO: 20 - protein, 8 aa, synthetic construct
  REGION 1..8: note = Linker
GGGSGGGS

SEQ ID NO: 21 - protein, 12 aa, synthetic construct
  REGION 1..12: note = Linker
GGGSGGGSGGGS

SEQ ID NO: 22 - protein, 16 aa, synthetic construct
  REGION 1..16: note = Linker
GGGSGGGSGGGSGGGS

SEQ ID NO: 23 - protein, 5 aa, synthetic construct
  REGION 1..5: note = Linker
GGGGS

SEQ ID NO: 24 - protein, 10 aa, synthetic construct
  REGION 1..10: note = Linker
GGGGSGGGGS

SEQ ID NO: 25 - protein, 15 aa, synthetic construct
  REGION 1..15: note = Linker
GGGGSGGGGSGGGGS

SEQ ID NO: 26 - protein, 20 aa, synthetic construct
  REGION 1..20: note = Linker
GGGGSGGGGSGGGGSGGGGS

SEQ ID NO: 27 - protein, 4 aa, synthetic construct
  REGION 1..4: note = Linker
SGSG

SEQ ID NO: 28 - protein, 7 aa, synthetic construct
  REGION 1..7: note = Linker
SGSGGSG

SEQ ID NO: 29 - protein, 10 aa, synthetic construct
  REGION 1..10: note = Linker
SGSGGSGGSG

SEQ ID NO: 30 - protein, 13 aa, synthetic construct
  REGION 1..13: note = Linker
SGSGGSGGSGGSG

SEQ ID NO: 31 - protein, 5 aa, synthetic construct
  REGION 1..5: note = Linker
SGGGS

SEQ ID NO: 32 - protein, 9 aa, synthetic construct
  REGION 1..9: note = Linker
SGGGSGGGS

SEQ ID NO: 33 - protein, 13 aa, synthetic construct
  REGION 1..13: note = Linker
SGGGSGGGSGGGS

SEQ ID NO: 34 - protein, 17 aa, synthetic construct
  REGION 1..17: note = Linker
SGGGSGGGSGGGSGGGS

SEQ ID NO: 35 - protein, 6 aa, synthetic construct
  REGION 1..6: note = Linker
SGGGGS

SEQ ID NO: 36 - protein, 11 aa, synthetic construct
  REGION 1..11: note = Linker
SGGGGSGGGGS

SEQ ID NO: 37 - protein, 16 aa, synthetic construct
  REGION 1..16: note = Linker
SGGGGSGGGGSGGGGS

SEQ ID NO: 38 - protein, 21 aa, synthetic construct
  REGION 1..21: note = Linker
SGGGGSGGGGSGGGGSGGGGS

SEQ ID NO: 39 - protein, 5 aa, synthetic construct
  REGION 1..5: note = Linker
EAAAK

SEQ ID NO: 40 - protein, 10 aa, synthetic construct
  REGION 1..10: note = Linker
EAAAKEAAAK

SEQ ID NO: 41 - protein, 15 aa, synthetic construct
  REGION 1..15: note = Linker
EAAAKEAAAKEAAAK

SEQ ID NO: 42 - protein, 20 aa, synthetic construct
  REGION 1..20: note = Linker
EAAAKEAAAKEAAAKEAAAK

SEQ ID NO: 43 - protein, 5 aa, synthetic construct
  REGION 1..5: note = Linker
PAPAP

SEQ ID NO: 44 - protein, 10 aa, synthetic construct
  REGION 1..10: note = Linker
PAPAPPAPAP

SEQ ID NO: 45 - protein, 15 aa, synthetic construct
  REGION 1..15: note = Linker
PAPAPPAPAPPAPAP

SEQ ID NO: 46 - protein, 20 aa, synthetic construct
  REGION 1..20: note = Linker
PAPAPPAPAPPAPAPPAPAP

SEQ ID NO: 47 - protein, 8 aa, synthetic construct
  REGION 1..8: note = Linker
IKRTVAAP

SEQ ID NO: 48 - protein, 5 aa, synthetic construct
  REGION 1..5: note = Linker
RAKPS

SEQ ID NO: 49 - protein, 10 aa, synthetic construct
  REGION 1..10: note = Linker
RAKPSRAKPS

SEQ ID NO: 50 - protein, 5 aa, synthetic construct
  REGION 1..5: note = Linker
ASTKG

SEQ ID NO: 51 - protein, 10 aa, synthetic construct
  REGION 1..10: note = Linker
ASTKGASTKG

SEQ ID NO: 52 - protein, 5 aa, synthetic construct
  REGION 1..5: note = Linker
AKTHT

SEQ ID NO: 53 - protein, 10 aa, synthetic construct
  REGION 1..10: note = Linker
AKTHTAKTHT

SEQ ID NO: 54 - protein, 28 aa, synthetic construct
  REGION 1..28: note = Linker
RRKRAPVKQTLNFDLLKLAGDVESNPGP

SEQ ID NO: 55 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 62 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 67 - protein, 9 aa, Homo sapiens
QRVSSSYLA

SEQ ID NO: 68 - protein, 7 aa, Homo sapiens
DASSRAT

SEQ ID NO: 69 - protein, 9 aa, Homo sapiens
QQYGSLPWT

SEQ ID NO: 70 - protein, 7 aa, Homo sapiens
GFTFSRY

SEQ ID NO: 71 - protein, 6 aa, Homo sapiens
KQDGSE

SEQ ID NO: 72 - protein, 12 aa, Homo sapiens
EGGWFGELAFDY

SEQ ID NO: 73 - protein, 108 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 74 - protein, 121 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 75 - protein, 215 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 451 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 8 aa, Homo sapiens
QDVSTAVA

SEQ ID NO: 78 - protein, 7 aa, Homo sapiens
SASFLYS

SEQ ID NO: 79 - protein, 9 aa, Homo sapiens
QQYLYHPAT

SEQ ID NO: 80 - protein, 7 aa, Homo sapiens
GFTFSDS

SEQ ID NO: 81 - protein, 6 aa, Homo sapiens
SPYGGS

SEQ ID NO: 82 - protein, 9 aa, Homo sapiens
RHWPGGFDY

SEQ ID NO: 83 - protein, 107 aa, Homo sapiens [sequence omitted]

SEQ ID NO: 84 - protein, 118 aa, Homo sapiens [sequence omitted]

SEQ ID NO: 85 - protein, 214 aa, Homo sapiens
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 448 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 11 aa, Homo sapiens
SSDVGGYNYVS

SEQ ID NO: 88 - protein, 7 aa, Homo sapiens
DVSNRPS

SEQ ID NO: 89 - protein, 10 aa, Homo sapiens
SSYTSSSTRV

SEQ ID NO: 90 - protein, 7 aa, Homo sapiens
GFTFSSY

SEQ ID NO: 91 - protein, 6 aa, Homo sapiens
YPSGGI

SEQ ID NO: 92 - protein, 11 aa, Homo sapiens
IKLGTVTTVDY

SEQ ID NO: 93 - protein, 110 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 94 - protein, 120 aa, Homo sapiens
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 95 - protein, 216 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 96 - protein, 450 aa, Homo sapiens
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 10 aa, Homo sapiens
ESVSIHGTHL

SEQ ID NO: 98 - protein, 7 aa, Homo sapiens
AASNLES

SEQ ID NO: 99 - protein, 9 aa, Homo sapiens
QQSFEDPLT

SEQ ID NO: 100 - protein, 8 aa, Homo sapiens
GYTFTSYW

SEQ ID NO: 101 - protein, 8 aa, Homo sapiens
IGPNSGFT

SEQ ID NO: 102 - protein, 12 aa, Homo sapiens
ARGGSSYDYFDY

SEQ ID NO: 103 - protein, 111 aa, Homo sapiens
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIK

SEQ ID NO: 104 - protein, 119 aa, Homo sapiens
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSS

SEQ ID NO: 105 - protein, 218 aa, Homo sapiens
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 446 aa, Homo sapiens
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 213 aa, Homo sapiens
YVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 108 - protein, 448 aa, Homo sapiens
EVQLLESGGGLVQPGGSLRLSCAASGFTFSSYAMSWVRQAPGKGLEWVSGISGSGGFTYYADSVKGRFTISRDNSKNTLYLQMNSLRAEDTAVYYCAKPPRGYNYGPFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 218 aa, Homo sapiens
NFMLTQPHSVSESPGKTVTISCTRSSGSIDSNYVQWYQQRPGSAPTTVIYEDNQRPSGVPDRFSGSIDSSSNSASLTISGLKTEDEADYYCQSYDSNNRHVIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 110 - protein, 450 aa, Homo sapiens
EVQLVQSGAEVKKPGSSVKVSCKASGGTFSRSAISWVRQAPGQGLEWMGVIIPAFGEANYAQKFQGRVTITADESTSTAYMELSSLRSEDTAVYYCARGRQMFGAGIDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 264 aa, Homo sapiens
QGQSGSGIALCPSHFCQLPQTGGGSSGGSGGSGGISSGLLSGRSDNHGGSDIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 443 aa, Homo sapiens
EVQLLESGGGLVQPGGSLRLSCAASGFTFSSYAMSWVRQAPGKGLEWVSSIWRNGIVTVYADSVKGRFTISRDNSKNTLYLQMNSLRAEDTAVYYCAKWSAAFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 113 - protein, 8 aa, Homo sapiens
GKMSSRRC

SEQ ID NO: 114 - protein, 8 aa, Homo sapiens
LLTTSGST

SEQ ID NO: 115 - protein, 21 aa, Homo sapiens
AADSFEDPTCTLVTSSGAFQY

SEQ ID NO: 116 - protein, 360 aa, Homo sapiens
QVQLVESGGGLVQPGGSLRLSCAASGKMSSRRCMAWFRQAPGKERERVAKLLTTSGSTYLADSVKGRFTISRDNSKNTVYLQMNSLRAEDTAVYYCAADSFEDPTCTLVTSSGAFQYWGQGTLVTVSSEPKSSDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAGIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 8 aa, Homo sapiens
GITFPVNA

SEQ ID NO: 118 - protein, 7 aa, Homo sapiens
ISAGGTT

SEQ ID NO: 119 - protein, 12 aa, Homo sapiens
YLQRRIGMLRDY

SEQ ID NO: 120 - protein, 25 aa, Homo sapiens
QVQLVESGGGLVQPGGSLRLSCAAS

SEQ ID NO: 121 - protein, 17 aa, Homo sapiens
YGWYRQAPGKQRDLVAI

SEQ ID NO: 122 - protein, 38 aa, Homo sapiens
NYADSVKGRFTISRDNSKNTLYLQMNSLRAEDTAVYYC

SEQ ID NO: 123 - protein, 11 aa, Homo sapiens
WGQGTQVTVSS

SEQ ID NO: 124 - protein, 118 aa, Homo sapiens
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSS

SEQ ID NO: 125 - protein, 270 aa, Homo sapiens
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 126 - protein, 176 aa, Homo sapiens
AFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 127 - protein, 176 aa, Homo sapiens
AFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 128 - protein, 159 aa, Homo sapiens
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 129 - protein, 159 aa, Homo sapiens
SITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 130 - protein, 303 aa, Homo sapiens
KFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRK

SEQ ID NO: 131 - protein, 179 aa, Homo sapiens
KFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTAT

SEQ ID NO: 132 - protein, 292 aa, Homo sapiens
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 133 - protein, 292 aa, Homo sapiens
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 134 - protein, 626 aa, Homo sapiens
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 135 - protein, 626 aa, Homo sapiens
EVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 136 - protein, 633 aa, Homo sapiens
KFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGGSGGGGSEVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSRRKRAPVKQTLNFDLLKLAGDVESNPGPAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 137 - protein, 633 aa, Homo sapiens
KFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGGSGGGGSEVQLVESGGGLVQPGGSLRLSCAASGSTWSINTLAWYRQAPGKQRDLVARISSGGSTYYADSVKGRFTISRDNSKNTLYLQMNSLRAEDTAVYYCYAQSTWYPPSWGQGTLVTVSSRRKRAPVKQTLNFDLLKLAGDVESNPGPAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 138 - protein, 419 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 139 - protein, 419 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 140 - protein, 535 aa, Homo sapiens
KFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 737 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 142 - protein, 737 aa, Homo sapiens; identical to SEQ ID NO: 141

SEQ ID NO: 143 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGA

SEQ ID NO: 144 - protein, 737 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 145 - protein, 239 aa, Homo sapiens
PRTHMSAPIENSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 419 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 147 - protein, 419 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 148 - protein, 545 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRK

SEQ ID NO: 149 - protein, 737 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 150 - protein, 737 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 151 - protein, 639 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 152 - protein, 639 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 153 - protein, 766 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRK

SEQ ID NO: 154 - protein, 958 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 155 - protein, 958 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 156 - protein, 448 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGA

SEQ ID NO: 157 - protein, 958 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 158 - protein, 958 aa, Homo sapiens
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 159 - protein, 214 aa, Homo sapiens; identical to SEQ ID NO: 85

SEQ ID NO: 160 - protein, 536 aa, Homo sapiens
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 161 - protein, 536 aa, Homo sapiens
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 162 - protein, 663 aa, Homo sapiens
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRK

SEQ ID NO: 163 - protein, 855 aa, Homo sapiens
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 164 - protein, 345 aa, Homo sapiens
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 855 aa, Homo sapiens
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 166 - protein, 345 aa, Homo sapiens
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGA

SEQ ID NO: 167 - protein, 855 aa, Homo sapiens
QVQLVESGGGLVQPGGSLRLSCAASGITFPVNAYGWYRQAPGKQRDLVAIISAGGTTNYADSVKGRFTISRDNSKNTLYLQMNSLRAEDTAVYYCYLQRRIGMLRDYWGQGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSKFSKQSWGLENEALIVRCPRQGKPSYTVDWYYSQTNKSIPTQERNRVFASGQLLKFLPAAVADSGIYTCIVRSPTFNRTGYANVTIYKKQSDCNVPDYLMYSTVSGSEKNSKIYCPTIDLYNWTAPLEWFKNCQALQGSRYRAHKSFLVIDNVMTEDAGDYTCKFIHNENGANYSVTATRSFTVKDEQGFSLFPVIGAPAQNEIKEVEIGKNANLTCSACFGKGTQFLAAVLWQLNGTKITDFGEPRIQQEEGQNQSFSNGLACLDMVLRIADVKEEDLLLQYDCLALNLHGLRRHTVRLSRKGGGSGGGSGGGSGGGSAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

(19) United States
(12) Patent Application Publication
(10) Pub. No.: US 2024/0327483 A1
(43) Pub. Date: Oct. 3, 2024

(54) FUSION PROTEINS COMPRISING SUPPRESSION OF TUMORIGENICITY 2 OR INTERLEUKIN-33, PHARMACEUTICAL COMPOSITIONS, AND THERAPEUTIC APPLICATIONS

(71) Applicant: Anwita Biosciences, Inc., San Carlos, CA (US)

(72) Inventors: Ziyang Zhong, Belmont, CA (US); Fan Ye, Mountain View, CA (US); Jianing Huang, San Mateo, CA (US)

(21) Appl. No.: 18/290,962

(22) PCT Filed: Jul. 23, 2022
(86) PCT No.: PCT/US2022/074089

§ 371 Date: Jan. 22, 2024

Related U.S. Application Data
(60) Provisional application No. 63/225,468, filed on Jul. 24, 2021.

(51) Int. Cl. C07K 14/54; A61K 38/00; A61K 39/00; A61P 35/00; C07K 14/82; C07K 16/28; C07K 16/30

(52) U.S. Cl. CPC C07K 14/54; A61P 35/00; C07K 14/82; C07K 16/2827; C07K 16/30; A61K 38/00; A61K 2039/505; C07K 2317/565; C07K 2317/569; C07K 2317/94; C07K 2319/30; C07K 2319/31

(57) ABSTRACT

Provided herein are fusion proteins comprising an ST2 domain or IL-33 domain or an ST2 domain, and a half-life-extension domain. Also provided herein are their pharmaceutical compositions and methods of use for treating, preventing, or ameliorating one or more symptoms of an IL-33-mediated disorder, disease, or condition.

### CROSS REFERENCE TO RELATED APPLICATION

This application claims the benefit of the priority of U.S. Provisional Application No. 63/225,468, filed Jul. 24, 2021; the disclosure of which is incorporated herein by reference in its entirety.

### FIELD

Provided herein are fusion proteins comprising an ST2 domain or IL-33 domain, and a half-life-extension domain. Also provided herein are their pharmaceutical compositions and methods of use for treating, preventing, or ameliorating one or more symptoms of an IL-33-mediated disorder, disease, or condition.

### REFERENCE TO A SEQUENCE LISTING

The present specification is being filed with a Sequence Listing entitled 216A014WO01_SEQLIST_ST26.XML of 195,027 bytes in size and created Jul. 23, 2022; the content of which is incorporated herein by reference in its entirety.

### BACKGROUND

Interleukin-33 (IL-33) is an interleukin-1 family cytokine. Sonnenberg et al., Nat. Immunol. 2011, 12, 383-90; Cohen et al., Nat. Commun. 2015, 6, 8327; Afferni et al., Front. Immunol. 2018, 9, Article 2601. IL-33 signals through a heterodimeric transmembrane receptor complex of IL-1 receptor-like 1 (IL1RL1), which is also known as suppression of tumorigenicity 2 (ST2), and IL-1 receptor accessory protein (IL1RAcP). Ali et al., Proc. Natl Acad. Sci. USA 2007, 104, 18660-5; Chackerian et al., J. Immunol. 2007, 179, 2551-5. The biological activity of IL-33 is regulated by several distinct mechanisms, including IL-33 inactivation by cysteine oxidation or proteolytic cleavage during apoptosis. Cohen et al., Nat. Commun. 2015, 6, 8327; Afferni et al., Front. Immunol. 2018, 9, Article 2601. Extracellular IL-33 is susceptible to cysteine oxidation that leads to the formation of disulfide, resulting in conformational changes that inhibit binding to an ST2 receptor. Cohen et al., Nat. Commun. 2015, 6, 8327.

Activation of ST2 signaling by IL-33 triggers pleiotropic immune responses in multiple ST2-expressing immune cells, including basophils, CD8⁺ T cells, eosinophils, macrophages, mast cells, neutrophils, NK cells, regulatory T cells, type 1 helper T cells, and type 2 helper T cells. Chen et al., Cell. Physiol. Biochem. 2018, 49, 349-58; Afferni et al., Front. Immunol. 2018, 9, Article 2601. IL-33 has been implicated in a wide variety of diseases, including allergic diseases, infectious diseases, cardiovascular diseases, chronic obstructive pulmonary disease (COPD), fibrotic diseases, musculoskeletal diseases, inflammatory bowel diseases, Alzheimer, obesity, diabetes, and cancer. Liew et al., Nat. Rev. Immunol. 2016, 16, 676-89; Afferni et al., Front. Immunol. 2018, 9, Article 2601. However, there is currently no FDA-approved drug that modulates IL-33 directly. Therefore, there is a need for an effective immunotherapy for treating an IL-33-mediated disorder, disease, or condition.

### SUMMARY OF THE DISCLOSURE

Provided herein is a fusion protein comprising a suppression of tumorigenicity 2 (ST2) domain or interleukin-33 (IL-33) domain, and a half-life-extension domain.

Also provided herein is a fusion protein comprising an ST2 domain or IL-33 domain, and an albumin binding domain.

Additionally provided herein is a fusion protein comprising an IL-33 domain, an albumin binding domain, and optionally a peptide linker; wherein the carboxy-terminus (C-terminus) of the IL-33 domain is connected to an amino-terminus (N-terminus) of the albumin binding domain directly or via the peptide linker; or wherein the N-terminus of the IL-33 domain is connected to a C-terminus of the albumin binding domain directly or via the peptide linker.

Furthermore, provided herein is a fusion protein comprising an ST2 domain, an albumin binding domain, and optionally a peptide linker; wherein the C-terminus of the ST2 domain is connected to an N-terminus of the albumin binding domain directly or via the peptide linker; or wherein the N-terminus of the ST2 domain is connected to a C-terminus of the albumin binding domain directly or via the peptide linker.

Provided herein is a fusion protein comprising an IL-33 domain, an ST2 domain, and an albumin binding domain.

Provided herein is a fusion protein comprising an IL-33 domain, an ST2 domain, an albumin binding domain, and optionally first and second peptide linkers; wherein the C-terminus of the ST2 domain is connected to an N-terminus of the albumin binding domain directly or via the first peptide linker, and a C-terminus of the albumin binding domain is connected to the N-terminus of the IL-33 domain directly or via the second peptide linker.

Provided herein is a fusion protein comprising an IL-33 domain, an ST2 domain, an albumin binding domain, and optionally first and second peptide linkers; wherein the C-terminus of the ST2 domain is connected to an N-terminus of the IL-33 domain directly or via the first peptide linker, and the C-terminus of the IL-33 domain is connected to an N-terminus of the albumin binding domain directly or via the second peptide linker.

Provided herein is a fusion protein comprising an IL-33 domain, an ST2 domain, an albumin binding domain, and optionally first and second peptide linkers; wherein a C-terminus of the albumin binding domain is connected to an N-terminus of the IL-33 domain directly or via the first peptide linker, and the C-terminus of the IL-33 domain is connected to the N-terminus of the ST2 domain directly or via the second peptide linker.

Provided herein is a fusion protein comprising an IL-33 domain, an ST2 domain, an albumin binding domain, and optionally first and second peptide linkers; wherein a C-terminus of the albumin binding domain is connected to the N-terminus of the ST2 domain directly or via the first peptide linker, and the C-terminus of the ST2 domain is connected to the N-terminus of the IL-33 domain directly or via the second peptide linker.

Provided herein is a fusion protein comprising an IL-33 domain, an ST2 domain, an albumin binding domain, and optionally first and second peptide linkers; wherein the C-terminus of the IL-33 is connected to an N-terminus of the albumin binding domain directly or via the first peptide linker, and a C-terminus of the albumin binding domain is connected to the N-terminus of the ST2 domain directly or via the second peptide linker.

Provided herein is a fusion protein comprising an IL-33 domain, an ST2 domain, an albumin binding domain, and optionally first and second peptide linkers; wherein the C-terminus of the IL-33 is connected to the N-terminus of the ST2 domain directly or via the first peptide linker, and the C-terminus of the ST2 domain is connected to an N-terminus of the albumin binding domain directly or via the second peptide linker.

Provided herein is a fusion protein comprising an ST2 domain or IL-33 domain, and a fragment crystallizable (Fc) domain.

Provided herein is a fusion protein comprising an IL-33 domain, an Fc domain comprising first and second peptide chains, and optionally a peptide linker; wherein the C-terminus of the IL-33 domain is connected to the N-terminus of the first peptide chain of the Fc domain directly or via the peptide linker, or the C-terminus of the first peptide chain of the Fc domain is connected to the N-terminus of the IL-33 domain directly or via the peptide linker.

Provided herein is a fusion protein comprising an ST2 domain, an Fc domain comprising first and second peptide chains, and optionally a peptide linker; wherein the C-terminus of the ST2 domain is connected to the N-terminus of the first peptide chain of the Fc domain directly or via the peptide linker, or the C-terminus of the first peptide chain of the Fc domain is connected to the N-terminus of the ST2 domain directly or via the peptide linker.

Provided herein is a fusion protein comprising first and second IL-33 domains, an Fc domain comprising first and second peptide chains, and optionally first and second peptide linkers; wherein the N-terminus of the first IL-33 domain is connected to the C-terminus of the first peptide chain of the Fc domain directly or via the first peptide linker, and the N-terminus of the second IL-33 domain is connected to the C-terminus of the second peptide chain of the Fc domain directly or via the second peptide linker.

Provided herein is a fusion protein comprising first and second ST2 domains, an Fc domain comprising first and second peptide chains, and optionally first and second peptide linkers; wherein the C-terminus of the first ST2 domain is connected to the N-terminus of the first peptide chain of the Fc domain directly or via the first peptide linker, and the C-terminus of the second ST2 domain is connected to the N-terminus of the second peptide chain of the Fc domain directly or via the second peptide linker.

Provided herein is a fusion protein comprising first and second ST2 domains, an Fc domain comprising first and second peptide chains, and optionally first and second peptide linkers; wherein the N-terminus of the first ST2 domain is connected to the C-terminus of the first peptide chain of the Fc domain directly or via the first peptide linker, and the N-terminus of the second ST2 domain is connected to the C-terminus of the second peptide chain of the Fc domain directly or via the second peptide linker.

Provided herein is a fusion protein comprising an IL-33 domain, an ST2 domain, and an Fc domain.

Provided herein is a fusion protein comprising an IL-33 domain, an ST2 domain, an Fc domain comprising first and second peptide chains, and optionally first and second peptide linkers; wherein the C-terminus of the first peptide chain of the Fc domain is connected to the N-terminus of the ST2 domain directly or via the first peptide linker, and the C-terminus of the ST2 domain is connected to the N-terminus of the IL-33 domain directly or via the second peptide linker.

Provided herein is a fusion protein comprising an IL-33 domain, an ST2 domain, an Fc domain comprising first and second peptide chains, and optionally first and second peptide linkers; wherein the C-terminus of the first peptide chain of the Fc domain is connected to the N-terminus of the IL-33 domain directly or via the first peptide linker, and the C-terminus of the second peptide chain of the Fc domain is connected to the N-terminus of the ST2 domain directly or via the second peptide linker.

Provided herein is a fusion protein comprising first and second IL-33 domains, first and second ST2 domains, an Fc domain comprising first and second peptide chains, and optionally first, second, third, and fourth peptide linkers; wherein the C-terminus of the first peptide chain of the Fc domain is connected to the N-terminus of the first ST2 domain directly or via the first peptide linker, and the C-terminus of the N-terminus of the first ST2 domain is connected to the N-terminus of the first IL-33 domain directly or via the second peptide linker; and wherein the C-terminus of the second peptide chain of the Fc domain is connected to the N-terminus of the second ST2 domain directly or via the third peptide linker, and the C-terminus of the N-terminus of the second ST2 domain is connected to the N-terminus of the second IL-33 domain directly or via the fourth peptide linker.

Provided herein is a fusion protein comprising an IL-33 domain, an ST2 domain, a programmed death-ligand 1 (PD-L1) binding domain, and an Fc domain.

Provided herein is a fusion protein comprising an IL-33 domain, an ST2 domain, first and second PD-L1 binding domains, an Fc domain comprising a first and second peptide chains, and optionally first and second peptide linkers, wherein a C-terminus of the first PD-L1 binding domain is connected to the N-terminus of the first peptide chain of the Fc domain directly or via a peptide linker, a C-terminus of the second PD-L1 binding domain is connected to the N-terminus of the second peptide chain of the Fc domain directly or via a peptide linker, the N-terminus of the ST2 domain is connected to the C-terminus of the first peptide chain of the Fc domain directly or via the first peptide linker, and the N-terminus of the IL-33 domain is connected to the C-terminus of the ST2 domain directly or via the second peptide linker.

Provided herein is a fusion protein comprising an IL-33 domain, an ST2 domain, first and second PD-L1 binding domains, an Fc domain comprising a first and second peptide chains, and optionally first and second peptide linkers; wherein a C-terminus of the first PD-L1 binding domain is connected to the N-terminus of the first peptide chain of the Fc domain directly or via a peptide linker, a C-terminus of the second PD-L1 binding domain is connected to the N-terminus of the second peptide chain of the Fc domain directly or via a peptide linker, the N-terminus of the IL-33 domain is connected to the C-terminus of the first peptide chain of the Fc domain directly or via the first peptide linker, and the N-terminus of the ST2 domain is connected to the C-terminus of the second peptide chain of the Fc domain directly or via the second peptide linker.

Provided herein is a fusion protein comprising first and second IL-33 domains, first and second ST2 domains, first and second PD-L1 binding domains, an Fc domain comprising a first and second peptide chains, and optionally first, second, third, and fourth peptide linkers; wherein a C-terminus of the first PD-L1 binding domain is connected to the N-terminus of the first peptide chain of the Fc domain directly or via a peptide linker, a C′-terminus of the second PD-L1 binding domain is connected to the N-terminus of the second peptide chain of the Fc domain directly or via a peptide linker; wherein the N-terminus of the first ST2 domain is connected to the C-terminus of the first peptide chain of the Fc domain directly or via the first peptide linker, and the N-terminus of the first IL-33 domain is connected to the C-terminus of the first ST2 domain directly or via the second peptide linker; and wherein the N-terminus of the second ST2 domain is connected to the C-terminus of the second peptide chain of the Fc domain directly or via the third peptide linker, and the N-terminus of the second IL-33 domain is connected to the C-terminus of the second ST2 domain directly or via the fourth peptide linker.

Provided herein is a fusion protein comprising an IL-33 domain, an ST2 domain, a mesothelin (MSLN) binding domain, and an Fc domain.

Provided herein is a fusion protein comprising an IL-33 domain, an ST2 domain, first and second MSLN binding domains, an Fc domain comprising a first and second peptide chains, and optionally first and second peptide linkers, wherein a C-terminus of the first MSLN binding domain is connected to the N-terminus of the first peptide chain of the Fc domain directly or via a peptide linker, a C-terminus of the second MSLN binding domain is connected to the N-terminus of the second peptide chain of the Fc domain directly or via a peptide linker, the N-terminus of the ST2 domain is connected to the C-terminus of the first peptide chain of the Fc domain directly or via the first peptide linker, and the N-terminus of the IL-33 domain is connected to the C-terminus of the ST2 domain directly or via the second peptide linker.

Provided herein is a fusion protein comprising an IL-33 domain, an ST2 domain, first and second MSLN binding domains, an Fc domain comprising a first and second peptide chains, and optionally first and second peptide linkers; wherein a C′-terminus of the first MSLN binding domain is connected to the N-terminus of the first peptide chain of the Fc domain directly or via a peptide linker, a C-terminus of the second MSLN binding domain is connected to the N-terminus of the second peptide chain of the Fc domain directly or via a peptide linker, the N-terminus of the IL-33 domain is connected to the C-terminus of the first peptide chain of the Fc domain directly or via the first peptide linker, and the N-terminus of the ST2 domain is connected to the C-terminus of the second peptide chain of the Fc domain directly or via the second peptide linker.

Provided herein is a fusion protein comprising first and second IL-33 domains, first and second ST2 domains, first and second MSLN binding domains, an Fc domain comprising a first and second peptide chains, and optionally first, second, third, and fourth peptide linkers; wherein a C-terminus of the first MSLN binding domain is connected to the N-terminus of the first peptide chain of the Fc domain directly or via a peptide linker, a C-terminus of the second MSLN binding domain is connected to the N-terminus of the second peptide chain of the Fc domain directly or via a peptide linker; wherein the N-terminus of the first ST2 domain is connected to the C-terminus of the first peptide chain of the Fc domain directly or via the first peptide linker, and the N-terminus of the first IL-33 domain is connected to the C-terminus of the first ST2 domain directly or via the second peptide linker; and wherein the N-terminus of the second ST2 domain is connected to the C-terminus of the second peptide chain of the Fc domain directly or via the third peptide linker, and the N-terminus of the second IL-33 domain is connected to the C-terminus of the second ST2 domain directly or via the fourth peptide linker.

Provided herein is an IL-33/ST2 complex comprising an IL-33 fusion protein provided herein and an ST2; wherein each IL-33 domain in the IL-33 fusion protein is complexed with an ST2.

Provided herein is an IL-33/ST2 complex comprising an ST2 fusion protein provided herein and an IL-33; wherein each ST2 domain in the ST2 fusion protein is complexed with an IL-33.

Provided herein is a pharmaceutical composition comprising a fusion protein or a fusion protein complex provided herein, and a pharmaceutically acceptable excipient.

Provided herein is a method of treating, preventing, or ameliorating one or more symptoms of an IL-33-mediated disorder, disease, or condition in a subject, comprising administering to the subject in need thereof a therapeutically effective amount of a fusion protein or fusion protein complex provided herein.

Provided herein is a method for treating, preventing, or ameliorating one or more symptoms of a proliferative disease in a subject, comprising administering to the subject in need thereof a therapeutically effective amount of a fusion protein or fusion protein complex provided herein.

Provided herein is a method for inhibiting the growth of a cell, comprising contacting the cell with an effective amount of a fusion protein or fusion protein complex provided herein.

### BRIEF DESCRIPTION OF THE DRAWINGS

FIG. 1 shows the configurations of exemplary fusion proteins: (i) an IL-33 fusion protein comprising an IL-33 domain and an albumin binding domain, e.g., an anti-HSA V_{H}H single domain antibody (sdAb), wherein the N-terminus of the IL-33 domain is connected to the C-terminus of the anti-HSA V_{H}H sdAb via a peptide linker; and (ii) an IL-33 fusion protein comprising first and second IL-33 domains and an Fc domain comprising first and second peptide chains, wherein the N-terminus of the first IL-33 domain is connected to the C-terminus of the first peptide chain of the Fc domain via a peptide linker, and the N-terminus of the second IL-33 domain is connected to the C-terminus of the second peptide chain of the Fc domain via a peptide linker.

FIG. 2 shows the configurations of exemplary fusion proteins and fusion protein complexes: (i) an IL-33/ST2 fusion protein comprising an IL-33 domain, an ST2 domain, and an albumin binding domain, e.g., an anti-HSA V_{H}H sdAb, wherein the N-terminus of IL-33 domain is connected to the C-terminus of the anti-HSA V_{H}H sdAb via a cleavable peptide linker, and the C-terminus of the ST2 domain is connected to the N-terminus of the anti-HSA V_{H}H sdAb via a non-cleavable peptide linker; and wherein, upon cleaved, the ST2 fusion protein formed forms an IL-33/ST2 complex with the cleaved IL-33 domain; (ii) an IL-33/ST2 fusion protein comprising an IL-33 domain, an ST2 domain, and an albumin binding domain, e.g., an anti-HSA V_{H}H sdAb, wherein the N-terminus of the IL-33 domain is connected to the C-terminus of the anti-HSA V_{H}H sdAb via a non-cleavable peptide linker, and the C-terminus of the ST2 domain is connected to the N-terminus of the anti-HSA V_{H}H sdAb via a cleavable peptide linker; and wherein, upon cleaved, the IL-33 fusion protein formed forms an IL-33/ST2 complex with the cleaved ST2 domain; (iii) an IL-33/ST2 fusion protein complex comprising: (a) an ST2 fusion protein that comprises an ST2 domain and an albumin binding domain, e.g., an anti-HSA V_{H}H sdAb, wherein the C-terminus of the ST2 domain is connected to the N-terminus of the anti-HSA V_{H}H sdAb via a peptide linker; and (b) a free IL-33; wherein the ST2 fusion protein forms an IL-33/ST2 complex with the free IL-33; (iv) an IL-33/ST2 fusion protein complex comprising: (a) an IL-33 fusion protein that comprises an IL-33 domain and an albumin binding domain, e.g., an anti-HSA V_{H}H sdAb, wherein the N-terminus of the IL-33 domain is connected to the C-terminus of the anti-HSA V_{H}H sdAb via a peptide linker; and (b) a free ST2; wherein the IL-33 fusion protein forms an IL-33/ST2 complex with the free ST2; and (v) an IL-33/ST2 fusion protein comprising an IL-33 domain, an ST2 domain, and an albumin binding domain, e.g., an anti-HSA V_{H}H sdAb, wherein the N-terminus of the IL-33 domain is connected to the C-terminus of the ST2-domain via a peptide linker, and the N-terminus of the ST2 domain is connected to the C-terminus of the anti-HSA V_{H}H sdAb via a peptide linker.

FIG. 3 shows the configurations of exemplary fusion proteins and a fusion protein complex: (i) an IL-33/ST2 fusion protein comprising an IL-33 domain, an ST2 domain, and an Fc domain comprising first and second peptide chains; wherein the N-terminus of the IL-33 domain is connected to the C-terminus of the ST2-domain via a peptide linker, and the N-terminus of the ST2 domain is connected to the C-terminus of the first peptide chain of the Fc domain via a peptide linker; (ii) an IL-33/ST2 fusion protein comprising an IL-33 domain, an ST2 domain, and an Fc domain comprising first and second peptide chains; wherein the N-terminus of the IL-33 domain is connected to the C-terminus of the first chain of the Fc domain via a peptide linker, and the N-terminus of the ST2 domain is connected to the C-terminus of the second chain of the Fc domain via a peptide linker; (iii) an IL-33/ST2 fusion protein comprising first and second IL-33 domains, first and second ST2 domains, and an Fc domain comprising first and second peptide chains; wherein the N-terminus of the first IL-33 domain is connected to the C-terminus of the first ST2-domain via a peptide linker, and the N-terminus of the first ST2 domain is connected to the C-terminus of the first peptide chain of the Fc domain via a peptide linker; and wherein the N-terminus of the second IL-33 domain is connected to the C-terminus of the second ST2-domain via a peptide linker, and the N-terminus of the second ST2 domain is connected to the C-terminus of the second peptide chain of the Fc domain via a peptide linker; (iv) an IL-33/ST2 fusion protein complex comprising: (a) an ST2 fusion protein that comprises first and second ST2 domains, and an Fc domain comprising first and second peptide chains; wherein the C-terminus of the first ST2 domain is connected to the N-terminus of the first peptide chain of the Fc domain directly or via a peptide linker, and the C-terminus of the second ST2 domain is connected to the N-terminus of the second peptide chain of the Fc domain directly or via a peptide linker; and (b) two free IL-33; wherein each ST2 domain is complexed with a free IL-33; and (v) an IL-33/ST2 fusion protein complex comprising: (a) an IL-33 fusion protein that comprises first and second IL-33 domains, and an Fc domain comprising first and second peptide chains; wherein the C-terminus of the first peptide chain of the Fc domain is connected to the N-terminus of the first IL-33 domain directly or via a peptide linker, and the C-terminus of the second peptide chain of the Fc domain is connected to the N-terminus of the second IL-33 domain directly or via a peptide linker; and (b) two free ST2; wherein each IL-33 domain is complexed with a free ST2.

FIG. 4 shows the configurations of exemplary fusion proteins: (i) an IL-33/ST2 fusion protein comprising an IL-33 domain, an ST2 domain, and an intact anti-PD-L1 antibody comprising first and second light chains and first and second heavy chains; wherein the N-terminus of the IL-33 domain is connected to the C′-terminus of the first heavy chain of the intact anti-PD-L1 antibody via a peptide linker, and the N-terminus of the ST2 domain is connected to the C-terminus of the second heavy chain of the intact anti-PD-L1 antibody via a peptide linker; (ii) an IL-33/ST2 fusion protein comprising an IL-33 domain, an ST2 domain, and an intact anti-PD-L1 antibody comprising first and second light chains and first and second heavy chains; wherein the N-terminus of the IL-33 domain is connected to the C-terminus of the ST2-domain via a peptide linker, and the N-terminus of the ST2 domain is connected to the C-terminus of the first heavy chain of the intact anti-PD-L1 antibody via a peptide linker; and (iii) an IL-33/ST2 fusion protein comprising first and second IL-33 domains, first and second ST2 domains, an intact anti-PD-L1 antibody comprising first and second light chains and first and second heavy chains: wherein the N-terminus of the first IL-33 domain is connected to the C-terminus of the first ST2-domain via a peptide linker, and the N-terminus of the first ST2 domain is connected to the C-terminus of the first heavy chain of the intact antibody via a peptide linker; and wherein the N-terminus of the second IL-33 domain is connected to the C-terminus of the second ST2-domain via a peptide linker, and the N-terminus of the second ST2 domain is connected to the C-terminus of the second heavy chain of the intact antibody via a peptide linker.

FIG. 5 shows the configurations of exemplary fusion proteins: (i) an IL-33/ST2 fusion protein comprising an IL-33 domain, an ST2 domain, two PD-L1 binding domains, e.g., two anti-PD-L1 V_{H}H sdAbs, and an Fc domain comprising first and second peptide chains; wherein the N-terminus of the IL-33 domain is connected to the C-terminus of the first peptide chain of the Fc domain via a peptide linker, and the N-terminus of the first peptide chain of the Fc domain is connected to the C-terminus of the first anti-PD-L1 V_{H}H sdAb directly or via a peptide linker; and wherein the N-terminus of the ST2 domain is connected to the C-terminus of the second peptide chain of the Fc domain via a peptide linker, and the N-terminus of the second peptide chain of the Fc domain is connected to the C-terminus of the second anti-PD-L1 V_{H}H sdAb directly or via a peptide linker; (ii) an IL-33/ST2 fusion protein comprising an IL-33 domain, an ST2 domain, two PD-L1 binding domains, e.g., two anti-PD-L1 V_{H}H sdAbs, and an Fc domain comprising first and second peptide chains; wherein the N-terminus of the IL-33 domain is connected to the C-terminus of the ST2 domain via a peptide linker, the N-terminus of the ST2 domain is connected to the C-terminus of the first peptide chain of the Fc domain via a peptide linker, and the N-terminus of the first peptide chain of the Fc domain is connected to the C-terminus of the first anti-PD-L1 V_{H}H sdAb directly or via a peptide linker; and wherein the N-terminus of the second peptide chain of the Fc domain is connected to the C-terminus of the second anti-PD-L1 V_{H}H sdAb directly or via a peptide linker; and (iii) an IL-33/ST2 fusion protein comprising first and second IL-33 domains, first and second ST2 domains, two PD-L1 binding domains, e.g., two anti-PD-L1 V_{H}H sdAbs, and an Fc domain comprising first and second peptide chains; wherein the N-terminus of the first IL-33 domain is connected to the C-terminus of the first ST2 domain via a peptide linker, the N-terminus of the first ST2 domain is connected to the C-terminus of the first peptide chain of the Fc domain via a peptide linker, and the N-terminus of the first peptide chain of the Fc domain is connected to the C-terminus of the first anti-PD-L1 V_{H}H sdAb directly or via a peptide linker; and wherein the N-terminus of the second IL-33 domain is connected to the C-terminus of the second ST2 domain via a peptide linker, the N-terminus of the second ST2 domain is connected to the C-terminus of the second peptide chain of the Fc domain via a peptide linker, and the N-terminus of the second peptide chain of the Fc domain is connected to the C-terminus of the second anti-PD-L1 V_{H}H sdAb directly or via a peptide linker.

FIG. 6 shows the configurations of exemplary fusion proteins: (i) an IL-33/ST2 fusion protein comprising an IL-33 domain, an ST2 domain, two MSLN binding domains, e.g., two anti-MSLN V_{H}H sdAbs, and an Fc domain comprising first and second peptide chains; wherein the N-terminus of the IL-33 domain is connected to the C-terminus of the first peptide chain of the Fc domain via a peptide linker, and the N-terminus of the first peptide chain of the Fc domain is connected to the C-terminus of the first anti-MSLN V_{H}H sdAb directly or via a peptide linker; and wherein the N-terminus of the ST2 domain is connected to the C-terminus of the second peptide chain of the Fc domain via a peptide linker, and the N-terminus of the second peptide chain of the Fc domain is connected to the C-terminus of the second anti-MSLN V_{H}H sdAb directly or via a peptide linker; (ii) an IL-33/ST2 fusion protein comprising an IL-33 domain, an ST2 domain, two MSLN binding domains, e.g., two anti-MSLN V_{H}H sdAbs, and an Fc domain comprising first and second peptide chains; wherein the N-terminus of the IL-33 domain is connected to the C-terminus of the ST2 domain via a peptide linker, the N-terminus of the ST2 domain is connected to the C-terminus of the first peptide chain of the Fc domain via a peptide linker, and the N-terminus of the first peptide chain of the Fc domain is connected to the C-terminus of the first anti-MSLN V_{H}H sdAb directly or via a peptide linker; and wherein the N-terminus of the second peptide chain of the Fc domain is connected to the C-terminus of the second anti-MSLN V_{H}H sdAb directly or via a peptide linker; and (iii) an IL-33/ST2 fusion protein comprising first and second IL-33 domains, first and second ST2 domains, two MSLN binding domains, e.g., two anti-MSLN V_{H}H sdAbs, and an Fc domain comprising first and second peptide chains; wherein the N-terminus of the first IL-33 domain is connected to the C-terminus of the first ST2 domain via a peptide linker, the N-terminus of the first ST2 domain is connected to the C-terminus of the first peptide chain of the Fc domain via a peptide linker, and the N-terminus of the first peptide chain of the Fc domain is connected to the C-terminus of the first anti-MSLN V_{H}H sdAb directly or via a peptide linker; and wherein the N-terminus of the second IL-33 domain is connected to the C-terminus of the second ST2 domain via a peptide linker, the N-terminus of the second ST2 domain is connected to the C-terminus of the second peptide chain of the Fc domain via a peptide linker, and the N-terminus of the second peptide chain of the Fc domain is connected to the C-terminus of the second anti-MSLN V_{H}H sdAb directly or via a peptide linker.

FIGS. 7A, 7B, and 7C show the SDS-PAGE protein characterization of anti-HSA-hIL-33 fusion protein A1 (SEQ ID NO: 132), anti-HSA-hST2-hIL-33 fusion protein A3 (SEQ ID NO: 134), and anti-HSA-hST2-hIL-33 mutein fusion protein A4 (SEQ ID NO: 135), respectively, under non-reducing conditions.

FIG. 8 shows the SDS-PAGE protein characterization of hIL-33/hST2 fusion protein complex C3 comprising anti-HSA-hIL-33 mutein fusion protein A2 (SEQ ID NO: 133) and free hST2 of SEQ ID NO: 130 under non-reducing conditions.

FIGS. 9A, 9B, and 9C show the SDS-PAGE protein characterization of hIgG1 Fc-hIL-33 fusion protein A7 (SEQ ID NO: 18), hIgG1 Fc-hIL-33 mutein fusion protein A8 (SEQ ID NO: 139), and hIgG1 Fc-hIL-33-hST2 fusion protein A10 (SEQ ID NOs: 141 and 143), respectively, under non-reducing conditions.

FIG. 10A shows the SDS-PAGE protein characterization of (i) hIL-33/hST2 fusion protein complex C10 comprising hST2-hIgG1 Fc A9 (SEQ ID NO: 140) and free hIL-33 mutein of SEQ ID NO: 127 under non-reducing conditions (Lane 1) and reducing conditions (Lane 2); and (ii) hIL-33/hST2 fusion protein complex C9 comprising hST2-hIgG1 Fc A9 (SEQ ID NO: 140) and free hIL-33 of SEQ ID NO: 126 under non-reducing conditions (Lane 3) and reducing conditions (Lane 4).

FIG. 10B shows the size exclusion chromatography (SEC) characterization of (i) fusion protein complex C10 comprising hST2-hIgG1 Fc A9 (SEQ ID NO: 140) and free hIL-33 mutein of SEQ ID NO: 127, and (ii) fusion protein complex C9 comprising hST2-hIgG1 Fc A9 (SEQ ID NO: 140) and free hIL-33 of SEQ ID NO: 126.

FIG. 11 shows the SDS-PAGE protein characterization of hIgG1 Fc-hST2-hIL-33 fusion protein A17 (SEQ ID NOs: 151, 153, and 159) under non-reducing conditions (Lane 1) and reducing conditions (Lane 2).

FIG. 12 shows the SDS-PAGE protein characterization of hIgG1 Fc-hST2-hIL-33 mutein fusion protein A18 (SEQ ID NOs: 152, 153, and 159) under non-reducing conditions (Lane 1) and reducing conditions (Lane 2).

FIG. 13 shows the antitumor effects of hIgG1 Fc-hST2-hIL-33 fusion protein A10 (SEQ ID NOs: 141 and 143) (19 μg) and anti-MSLN-hST2-hIL-33 fusion protein A25 (SEQ ID NOs: 163 and 164) (23 μg) in combination with anti-TRP1 antibody TA99 (100 μg) in a xenograft mouse model.

FIG. 14 shows the antitumor effects of anti-PD-L1 antibody atezolizumab (SEQ ID NOs: 75 and 76) (25 μg) and its corresponding fusion protein (hIgG1 Fc-hST2-hIL-33 mutein fusion protein A18 (SEQ ID NOs: 152, 153, and 159)) (35 μg) in a xenograft mouse model.

### DETAILED DESCRIPTION

To facilitate understanding of the disclosure set forth herein, a number of terms are defined below.

Generally, the nomenclature used herein and the laboratory procedures in biochemistry, biology, cell biology, immunology, molecular biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The term “therapeutically effective amount” or “effective amount” is meant to include the amount of a compound (e.g., a polypeptide, fusion protein, or fusion protein complex) that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers to an amount, concentration, or dosage of a compound (e.g., a polypeptide, fusion protein, or fusion protein complex) that is required for 50% inhibition of a maximal response in an assay that measures such a response.

The term “pharmaceutically acceptable carrier.” “pharmaceutically acceptable excipient,” “physiologically acceptable carrier.” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human or an animal) without excessive toxicity, irritation, allergic response, immunogenicity, or other problems or complications, commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical Preformulation and Formulation, 1st ed.; Gibson Ed.; CRC Press, 2015.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “substantially pure” and “substantially homogeneous,” when referring to a compound (e.g., a polypeptide, fusion protein, or fusion protein complex), mean sufficiently homogeneous to appear free of readily detectable impurities as determined by standard analytical methods used by one of ordinary skill in the art, including, but not limited to, gel electrophoresis, high performance liquid chromatography (HPLC), and mass spectrometry (MS); or sufficiently pure such that further purification would not detectably alter the physical, chemical, biological, and/or pharmacological properties, such as enzymatic and biological activities, of the compound. In certain embodiments, “substantially pure” or “substantially homogeneous” refers to a collection of molecules, wherein at least about 50%, at least about 70%, at least about 80%, at least about 90%, at least about 95%, at least about 98%, at least about 99%, or at least about 99.5% by weight of the molecules are a single compound or a complex as determined by a standard analytical method.

### Fusion Proteins

In one embodiment, provided herein is a fusion protein comprising an interleukin-33 (IL-33) domain or a suppression of tumorigenicity 2 (ST2) domain, and a half-life-extension domain.

In another embodiment, provided herein is a fusion protein comprising an IL-33 domain and a half-life-extension domain.

In yet another embodiment, provided herein is a fusion protein comprising an ST2 domain and a half-life-extension domain.

In one embodiment, the half-life-extension domain comprises an albumin binding domain, a constant region of an antibody, a constant region of a heavy chain of an antibody, a fragment crystallizable (Fc) domain, a serum albumin, a polyethylene glycol (PEG) group, or a fatty acyl group. In another embodiment, the half-life-extension domain is an albumin binding domain. In yet another embodiment, the half-life-extension domain is a constant region of an antibody comprising two light chains and two heavy chains: C_{L} plus C_{H}1, C_{H}2, and C_{H}3. In yet another embodiment, the half-life-extension domain is a constant region of a heavy chain (C_{H}1, C_{H}2, and C_{H}3) of an antibody. In yet another embodiment, the half-life-extension domain is an Fc domain. In yet another embodiment, the half-life-extension domain is an Fc domain comprising first and second peptide chains. In yet another embodiment, the half-life-extension domain is a serum albumin. In yet another embodiment, the half-life-extension domain is a PEG group. In still another embodiment, the half-life-extension domain is a fatty acyl group.

In certain embodiments, the half-life-extension domain extends the half-life of the IL-33 domain in vivo as compared to the corresponding free IL-33 polypeptide.

In one embodiment, provided herein is a fusion protein comprising an IL-33 domain or an ST2 domain, and an albumin binding domain.

In another embodiment, provided herein is a fusion protein comprising an IL-33 domain and an albumin binding domain.

In one embodiment, the fusion protein provided herein comprises an IL-33 domain, an albumin binding domain, and optionally a peptide linker; wherein the carboxy-terminus (C-terminus) of the IL-33 domain is connected to an amino-terminus (N-terminus) of the albumin binding domain directly or via the peptide linker; or wherein the N-terminus of the IL-33 domain is connected to a C-terminus of the albumin binding domain directly or via the peptide linker.

In another embodiment, the fusion protein provided herein comprises an IL-33 domain, an albumin binding domain, and optionally a peptide linker; wherein the C-terminus of the IL-33 domain is connected to an N-terminus of the albumin binding domain directly or via the peptide linker.

In yet another embodiment, the fusion protein provided herein comprises an IL-33 domain, an albumin binding domain, and a peptide linker; wherein the C-terminus of the IL-33 domain is connected to an N-terminus of the albumin binding domain via the peptide linker.

In yet another embodiment, the fusion protein provided herein comprises an IL-33 domain, an albumin binding domain, and optionally a peptide linker; wherein the N-terminus of the IL-33 domain is connected to a C-terminus of the albumin binding domain directly or via the peptide linker.

In still another embodiment, the fusion protein provided herein comprises an IL-33 domain, an albumin binding domain, and a peptide linker; wherein the N-terminus of the IL-33 domain is connected to a C-terminus of the albumin binding domain via the peptide linker.

In yet another embodiment, provided herein is a fusion protein comprising an ST2 domain and an albumin binding domain.

In one embodiment, the fusion protein provided herein comprises an ST2 domain, an albumin binding domain, and optionally a peptide linker; wherein the C-terminus of the ST2 domain is connected to an N-terminus of the albumin binding domain directly or via the peptide linker; or wherein the N-terminus of the ST2 domain is connected to a C-terminus of the albumin binding domain directly or via the peptide linker.

In another embodiment, the fusion protein provided herein comprises an ST2 domain, an albumin binding domain, and optionally a peptide linker; wherein the C-terminus of the ST2 domain is connected to an N-terminus of the albumin binding domain directly or via the peptide linker.

In yet another embodiment, the fusion protein provided herein comprises an ST2 domain, an albumin binding domain, and a peptide linker; wherein the C-terminus of the ST2 domain is connected to an N-terminus of the albumin binding domain via the peptide linker.

In yet another embodiment, the fusion protein provided herein comprises an ST2 domain, an albumin binding domain, and optionally a peptide linker; wherein the N-terminus of the ST2 domain is connected to a C-terminus of the albumin binding domain directly or via the peptide linker.

In still another embodiment, the fusion protein provided herein comprises an ST2 domain, an albumin binding domain, and a peptide linker; wherein the N-terminus of the ST2 domain is connected to a C-terminus of the albumin binding domain via the peptide linker.

In still another embodiment, provided herein is a fusion protein comprising an IL-33 domain, an ST2 domain, and an albumin binding domain.

In one embodiment, the fusion protein provided herein comprises an IL-33 domain, an ST2 domain, an albumin binding domain, and optionally first and second peptide linkers; wherein the C-terminus of the ST2 domain is connected to an N-terminus of the albumin binding domain directly or via the first peptide linker, and a C-terminus of the albumin binding domain is connected to the N-terminus of the IL-33 domain directly or via the second peptide linker.

In another embodiment, the fusion protein provided herein comprises an IL-33 domain, an ST2 domain, an albumin binding domain, and first and second peptide linkers; wherein the C-terminus of the ST2 domain is connected to an N-terminus of the albumin binding domain via the first peptide linker, and a C-terminus of the albumin binding domain is connected to the N-terminus of the IL-33 domain via the second peptide linker.

In yet another embodiment, the fusion protein provided herein comprises an IL-33 domain, an ST2 domain, an albumin binding domain, and optionally first and second peptide linkers; wherein the C-terminus of the ST2 domain is connected to the N-terminus of the IL-33 domain directly or via the first peptide linker, and the C-terminus of the IL-33 domain is connected to an N-terminus of the albumin binding domain directly or via the second peptide linker.

In yet another embodiment, the fusion protein provided herein comprises an IL-33 domain, an ST2 domain, an albumin binding domain, and first and second peptide linkers; wherein the C-terminus of the ST2 domain is connected to the N-terminus of the IL-33 domain via the first peptide linker, and the C-terminus of the IL-33 domain is connected to an N-terminus of the albumin binding domain via the second peptide linker.

In yet another embodiment, the fusion protein provided herein comprises an IL-33 domain, an ST2 domain, an albumin binding domain, and optionally first and second peptide linkers; wherein a C-terminus of the albumin binding domain is connected to the N-terminus of the IL-33 domain directly or via the first peptide linker, and the C-terminus of the IL-33 domain is connected to the N-terminus of the ST2 domain directly or via the second peptide linker.

In yet another embodiment, the fusion protein provided herein comprises an IL-33 domain, an ST2 domain, an albumin binding domain, and first and second peptide linkers; wherein a C-terminus of the albumin binding domain is connected to the N-terminus of the IL-33 domain via the first peptide linker, and the C-terminus of the IL-33 domain is connected to the N-terminus of the ST2 domain via the second peptide linker.

In yet another embodiment, the fusion protein provided herein comprises an IL-33 domain, an ST2 domain, an albumin binding domain, and optionally first and second peptide linkers; wherein a C-terminus of the albumin binding domain is connected to the N-terminus of the ST2 domain directly or via the first peptide linker, and the C-terminus of the ST2 domain is connected to the N-terminus of the IL-33 domain directly or via the second peptide linker.

In yet another embodiment, the fusion protein provided herein comprises an IL-33 domain, an ST2 domain, an albumin binding domain, and first and second peptide linkers; wherein a C-terminus of the albumin binding domain is connected to the N-terminus of the ST2 domain via the first peptide linker, and the C-terminus of the ST2 domain is connected to the N-terminus of the IL-33 domain via the second peptide linker.

In yet another embodiment, the fusion protein provided herein comprises an IL-33 domain, an ST2 domain, an albumin binding domain, and optionally first and second peptide linkers; wherein the C-terminus of the IL-33 is connected to an N-terminus of the albumin binding domain directly or via the first peptide linker, and a C-terminus of the albumin binding domain is connected to the N-terminus of the ST2 domain directly or via the second peptide linker.

In yet another embodiment, the fusion protein provided herein comprises an IL-33 domain, an ST2 domain, an albumin binding domain, and first and second peptide linkers; wherein the C-terminus of the IL-33 is connected to an N-terminus of the albumin binding domain via the first peptide linker, and a C-terminus of the albumin binding domain is connected to the N-terminus of the ST2 domain via the second peptide linker.

In yet another embodiment, the fusion protein provided herein comprises an IL-33 domain, an ST2 domain, an albumin binding domain, and optionally first and second peptide linkers; wherein the C-terminus of the IL-33 is connected to the N-terminus of the ST2 domain directly or via the first peptide linker, and the C-terminus of the ST2 domain is connected to an N-terminus of the albumin binding domain directly or via the second peptide linker.

In still another embodiment, the fusion protein provided herein comprises an IL-33 domain, an ST2 domain, an albumin binding domain, and optionally first and second peptide linkers; wherein the C-terminus of the IL-33 is connected to the N-terminus of the ST2 domain via the first peptide linker, and the C-terminus of the ST2 domain is connected to an N-terminus of the albumin binding domain via the second peptide linker.

In one embodiment, the albumin binding domain is an antibody or a fragment thereof that binds to an albumin. In another embodiment, the albumin binding domain is an antibody or a fragment thereof that binds to a human serum albumin (HSA). In yet another embodiment, the albumin binding domain is an antibody or a fragment thereof that binds to an HSA specifically.

In certain embodiments, the anti-HSA antibody binds to an HSA with a K_{d} ranging from about 10 pM to about 1,000 nM, from about 1 to about 500 nM, from about 1 to about 200 nM, or from about 1 to about 100 nM at a pH of about 7. In certain embodiments, the anti-HSA antibody binds to an HSA with a K_{d} ranging from about 10 pM to about 1,000 nM at a pH of about 7. In certain embodiments, the anti-HSA antibody binds to an HSA with a K_{d} ranging from about 1 nM to about 500 nM at a pH of about 7. In certain embodiments, the anti-HSA antibody binds to an HSA with a K_{d} ranging from about 1 nM to about 200 nM at a pH of about 7. In certain embodiments, the anti-HSA antibody binds to an HSA with a K_{d} ranging from about 1 nM to about 100 nM at a pH of about 7.

In one embodiment, the albumin binding domain is an anti-HSA antibody or a fragment thereof, comprising (i) a complementarity determining region 1 (CDR1) of SEQ ID NO: 1, a complementarity determining region 2 (CDR2) of SEQ ID NO: 2, and a complementarity determining region 3 (CDR3) of SEQ ID NO: 3; or (ii) a CDR1 of SEQ ID NO: 9, a CDR2 of SEQ ID NO: 10, and a CDR3 of SEQ ID NO: 11. In another embodiment, the albumin binding domain is an anti-HSA antibody or a fragment thereof, comprising a CDR1 of SEQ ID NO: 1, a CDR2 of SEQ ID NO: 2, and a CDR3 of SEQ ID NO: 3. In yet another embodiment, the albumin binding domain comprises a CDR1 of SEQ ID NO: 9, a CDR2 of SEQ ID NO: 10, and a CDR3 of SEQ ID NO: 11.

In one embodiment, the albumin binding domain is an anti-HSA antibody or a fragment thereof, comprising an amino acid sequence of SEQ ID NO: 8 or 15. In another embodiment, the albumin binding domain is an anti-HSA antibody or a fragment thereof, comprising an amino acid sequence of SEQ ID NO: 8. In still another embodiment, the albumin binding domain comprises an amino acid sequence of SEQ ID NO: 15.

In certain embodiments, the albumin binding domain is an anti-HSA antibody disclosed in WO 2019/246004 A1 or WO 2020/172528 A1, the disclosure of each of which is incorporated herein by reference in its entirety.

In certain embodiments, the anti-HSA antibody is a human antibody. In certain embodiments, the anti-HSA antibody is a humanized antibody.

In one embodiment, provided herein is a fusion protein comprising an IL-33 domain or an ST2 domain, and an anti-HSA antibody.

In another embodiment, provided herein is a fusion protein comprising an IL-33 domain and an anti-HSA antibody.

In one embodiment, the fusion protein provided herein comprises an IL-33 domain, an anti-HSA antibody, and optionally a peptide linker; wherein the C-terminus of the IL-33 domain is connected to an N-terminus of the anti-HSA antibody directly or via the peptide linker; or wherein the N-terminus of the IL-33 domain is connected to a C-terminus of the anti-HSA antibody directly or via the peptide linker.

In another embodiment, the fusion protein provided herein comprises an IL-33 domain, an anti-HSA antibody, and optionally a peptide linker; wherein the C-terminus of the IL-33 domain is connected to an N-terminus of the anti-HSA antibody directly or via the peptide linker.

In yet another embodiment, the fusion protein provided herein comprises an IL-33 domain, an anti-HSA antibody, and a peptide linker; wherein the C-terminus of the IL-33 domain is connected to an N-terminus of the anti-HSA antibody via the peptide linker.

In yet another embodiment, the fusion protein provided herein comprises an IL-33 domain, an anti-HSA antibody, and optionally a peptide linker; wherein the N-terminus of the IL-33 domain is connected to a C-terminus of the anti-HSA antibody directly or via the peptide linker.

In still another embodiment, the fusion protein provided herein comprises an IL-33 domain, an anti-HSA antibody, and a peptide linker; wherein the N-terminus of the IL-33 domain is connected to a C-terminus of the anti-HSA antibody via the peptide linker.

In yet another embodiment, provided herein is a fusion protein comprising an ST2 domain and an anti-HSA antibody.

In one embodiment, the fusion protein provided herein comprises an ST2 domain, an anti-HSA antibody, and optionally a peptide linker; wherein the C-terminus of the ST2 domain is connected to an N-terminus of the anti-HSA antibody directly or via the peptide linker; or wherein the N-terminus of the ST2 domain is connected to a C-terminus of the anti-HSA antibody directly or via the peptide linker.

In another embodiment, the fusion protein provided herein comprises an ST2 domain, an anti-HSA antibody, and optionally a peptide linker; wherein the C-terminus of the ST2 domain is connected to an N-terminus of the anti-HSA antibody directly or via the peptide linker.

In yet another embodiment, the fusion protein provided herein comprises an ST2 domain, an anti-HSA antibody, and a peptide linker; wherein the C-terminus of the ST2 domain is connected to an N-terminus of the anti-HSA antibody via the peptide linker.

In yet another embodiment, the fusion protein provided herein comprises an ST2 domain, an anti-HSA antibody, and optionally a peptide linker; wherein the N-terminus of the ST2 domain is connected to a C-terminus of the anti-HSA antibody directly or via the peptide linker.

In still another embodiment, the fusion protein provided herein comprises an ST2 domain, an anti-HSA antibody, and a peptide linker; wherein the N-terminus of the ST2 domain is connected to a C-terminus of the anti-HSA antibody via the peptide linker.

In still another embodiment, provided herein is a fusion protein comprising an IL-33 domain, an ST2 domain, and an anti-HSA antibody.

In one embodiment, the fusion protein provided herein comprises an IL-33 domain, an ST2 domain, an anti-HSA antibody, and optionally first and second peptide linkers; wherein the C-terminus of the ST2 domain is connected to an N-terminus of the anti-HSA antibody directly or via the first peptide linker, and a C-terminus of the anti-HSA antibody is connected to the N-terminus of the IL-33 domain directly or via the second peptide linker.

In another embodiment, the fusion protein provided herein comprises an IL-33 domain, an ST2 domain, an anti-HSA antibody, and first and second peptide linkers; wherein the C-terminus of the ST2 domain is connected to an N-terminus of the anti-HSA antibody via the first peptide linker, and a C-terminus of the anti-HSA antibody is connected to the N-terminus of the IL-33 domain via the second peptide linker.

In yet another embodiment, the fusion protein provided herein comprises an IL-33 domain, an ST2 domain, an anti-HSA antibody, and optionally first and second peptide linkers; wherein the C-terminus of the ST2 domain is connected to the N-terminus of the IL-33 domain directly or via the first peptide linker, and the C-terminus of the IL-33 domain is connected to an N-terminus of the anti-HSA antibody directly or via the second peptide linker.

In yet another embodiment, the fusion protein provided herein comprises an IL-33 domain, an ST2 domain, an anti-HSA antibody, and first and second peptide linkers; wherein the C-terminus of the ST2 domain is connected to the N-terminus of the IL-33 domain via the first peptide linker, and the C-terminus of the IL-33 domain is connected to an N-terminus of the anti-HSA antibody via the second peptide linker.

In yet another embodiment, the fusion protein provided herein comprises an IL-33 domain, an ST2 domain, an anti-HSA antibody, and optionally first and second peptide linkers; wherein a C-terminus of the anti-HSA antibody is connected to the N-terminus of the IL-33 domain directly or via the first peptide linker, and the C-terminus of the IL-33 domain is connected to the N-terminus of the ST2 domain directly or via the second peptide linker.

In yet another embodiment, the fusion protein provided herein comprises an IL-33 domain, an ST2 domain, an anti-HSA antibody, and first and second peptide linkers; wherein a C-terminus of the anti-HSA antibody is connected to the N-terminus of the IL-33 domain via the first peptide linker, and the C-terminus of the IL-33 domain is connected to the N-terminus of the ST2 domain via the second peptide linker.

In yet another embodiment, the fusion protein provided herein comprises an IL-33 domain, an ST2 domain, an anti-HSA antibody, and optionally first and second peptide linkers; wherein a C-terminus of the anti-HSA antibody is connected to the N-terminus of the ST2 domain directly or via the first peptide linker, and the C-terminus of the ST2 domain is connected to the N-terminus of the IL-33 domain directly or via the second peptide linker.

In yet another embodiment, the fusion protein provided herein comprises an IL-33 domain, an ST2 domain, an anti-HSA antibody, and first and second peptide linkers; wherein a C-terminus of the anti-HSA antibody is connected to the N-terminus of the ST2 domain via the first peptide linker, and the C-terminus of the ST2 domain is connected to the N-terminus of the IL-33 domain via the second peptide linker.

In yet another embodiment, the fusion protein provided herein comprises an IL-33 domain, an ST2 domain, an anti-HSA antibody, and optionally first and second peptide linkers; wherein the C-terminus of the IL-33 is connected to an N-terminus of the anti-HSA antibody directly or via the first peptide linker, and a C-terminus of the anti-HSA antibody is connected to the N-terminus of the ST2 domain directly or via the second peptide linker.

In yet another embodiment, the fusion protein provided herein comprises an IL-33 domain, an ST2 domain, an anti-HSA antibody, and first and second peptide linkers; wherein the C-terminus of the IL-33 is connected to an N-terminus of the anti-HSA antibody via the first peptide linker, and a C-terminus of the anti-HSA antibody is connected to the N-terminus of the ST2 domain via the second peptide linker.

In yet another embodiment, the fusion protein provided herein comprises an IL-33 domain, an ST2 domain, an anti-HSA antibody, and optionally first and second peptide linkers; wherein the C-terminus of the IL-33 is connected to the N-terminus of the ST2 domain directly or via the first peptide linker, and the C-terminus of the ST2 domain is connected to an N-terminus of the anti-HSA antibody directly or via the second peptide linker.

In still another embodiment, the fusion protein provided herein comprises an IL-33 domain, an ST2 domain, an anti-HSA antibody, and first and second peptide linkers; wherein the C-terminus of the IL-33 is connected to the N-terminus of the ST2 domain via the first peptide linker, and the C-terminus of the ST2 domain is connected to an N-terminus of the anti-HSA antibody via the second peptide linker.

In certain embodiments, the anti-HSA antibody is an anti-HSA single domain antibody (sdAb).

In certain embodiments, the anti-HSA sdAb binds to an HSA with a K_{d} ranging from about 10 pM to about 1,000 nM, from about 1 to about 500 nM, from about 1 to about 200 nM, or from about 1 to about 100 nM at a pH of about 7. In certain embodiments, the anti-HSA sdAb binds to an HSA with a K_{d} ranging from about 10 pM to about 1,000 nM at a pH of about 7. In certain embodiments, the anti-HSA sdAb binds to an HSA with a K_{d} ranging from about 1 to about 500 nM at a pH of about 7. In certain embodiments, the anti-HSA sdAb binds to an HSA with a K_{d} ranging from about 1 to about 200 nM at a pH of about 7. In certain embodiments, the anti-HSA sdAb binds to an HSA with a K_{d} ranging from about 1 to about 100 nM at a pH of about 7.

In one embodiment, the anti-HSA sdAb comprises (i) a CDR1 of SEQ ID NO: 1, a CDR2 of SEQ ID NO: 2, and a CDR3 of SEQ ID NO: 3; or (ii) a CDR1 of SEQ ID NO: 9, a CDR2 of SEQ ID NO: 10, and a CDR3 of SEQ ID NO: 11. In another embodiment, the anti-HSA sdAb comprises a CDR1 of SEQ ID NO: 1, a CDR2 of SEQ ID NO: 2, and a CDR3 of SEQ ID NO: 3. In yet another embodiment, the anti-HSA sdAb comprises a CDR1 of SEQ ID NO: 9, a CDR2 of SEQ ID NO: 10, and a CDR3 of SEQ ID NO: 11.

In one embodiment, the anti-HSA sdAb has the structure of FR1-CDR1-FR2-CDR2-FR3-CDR3-FR4, wherein:
CDR1, CDR2, and CDR3 are:
   (i) CDR1 of SEQ ID NO: 1, CDR2 of SEQ ID NO: 2, and CDR3 of SEQ ID NO: 3; or
   (ii) CDR1 of SEQ ID NO: 9, CDR2 of SEQ ID NO: 10, and CDR3 of SEQ ID NO: 11;
FR1 is an amino acid sequence of SEQ ID NO: 4 or 12;
FR2 is an amino acid sequence of SEQ ID NO: 5 or 13;
FR3 is an amino acid sequence of SEQ ID NO: 6; and
FR4 is an amino acid sequence of SEQ ID NO: 7 or 14.

In another embodiment, the anti-HSA sdAb has the structure of FR1-CDR1-FR2-CDR2-FR3-CDR3-FR4, wherein:
CDR1, CDR2, and CDR3 are:
   (i) CDR1 of SEQ ID NO: 1, CDR2 of SEQ ID NO: 2, and CDR3 of SEQ ID NO: 3; or
   (ii) CDR1 of SEQ ID NO: 9, CDR2 of SEQ ID NO: 10, and CDR3 of SEQ ID NO: 11;
FR1 is an amino acid sequence of SEQ ID NO: 4;
FR2 is an amino acid sequence of SEQ ID NO: 5;
FR3 is an amino acid sequence of SEQ ID NO: 6; and
FR3 is an amino acid sequence of SEQ ID NO: 7.

In yet another embodiment, the anti-HSA sdAb has the structure of FR1-CDR1-FR2-CDR2-FR3-CDR3-FR4, wherein:
CDR1, CDR2, and CDR3 are:
   (i) CDR1 of SEQ ID NO: 1, CDR2 of SEQ ID NO: 2, and CDR3 of SEQ ID NO: 3; or
   (ii) CDR1 of SEQ ID NO: 9, CDR2 of SEQ ID NO: 10, and CDR3 of SEQ ID NO: 11;
FR1 is an amino acid sequence of SEQ ID NO: 12;
FR2 is an amino acid sequence of SEQ ID NO: 13;
FR3 is an amino acid sequence of SEQ ID NO: 6; and
FR3 is an amino acid sequence of SEQ ID NO: 14.

In one embodiment, the anti-HSA sdAb has an amino acid sequence of SEQ ID NO: 8 or 15. In another embodiment, the anti-HSA sdAb has an amino acid sequence of SEQ ID NO: 8. In yet another embodiment, the anti-HSA sdAb has an amino acid sequence of SEQ ID NO: 15.

In certain embodiments, the anti-HSA sdAb is one disclosed in WO 2019/246004 A1 or WO 2020/172528 A1, the disclosure of each of which is incorporated herein by reference in its entirety.

In certain embodiments, the anti-HSA sdAb is a human antibody. In certain embodiments, the anti-HSA sdAb is a humanized antibody.

In one embodiment, provided herein is a fusion protein comprising an IL-33 domain or an ST2 domain, and an anti-HSA sdAb.

In another embodiment, provided herein is a fusion protein comprising an IL-33 domain and an anti-HSA sdAb.

In one embodiment, the fusion protein provided herein comprises an IL-33 domain, an anti-HSA sdAb, and optionally a peptide linker; wherein the C-terminus of the IL-33 domain is connected to the N-terminus of the anti-HSA sdAb directly or via the peptide linker; or wherein the N-terminus of the IL-33 domain is connected to the C-terminus of the anti-HSA sdAb directly or via the peptide linker.

In another embodiment, the fusion protein provided herein comprises an IL-33 domain, an anti-HSA sdAb, and optionally a peptide linker; wherein the C-terminus of the IL-33 domain is connected to the N-terminus of the anti-HSA sdAb directly or via the peptide linker.

In yet another embodiment, the fusion protein provided herein comprises an IL-33 domain, an anti-HSA sdAb, and a peptide linker; wherein the C-terminus of the IL-33 domain is connected to the N-terminus of the anti-HSA sdAb via the peptide linker.

In yet another embodiment, the fusion protein provided herein comprises an IL-33 domain, an anti-HSA sdAb, and optionally a peptide linker; wherein the N-terminus of the IL-33 domain is connected to the C-terminus of the anti-HSA sdAb directly or via the peptide linker.

In still another embodiment, the fusion protein provided herein comprises an IL-33 domain, an anti-HSA sdAb, and a peptide linker; wherein the N-terminus of the IL-33 domain is connected to the C-terminus of the anti-HSA sdAb via the peptide linker.

In yet another embodiment, provided herein is a fusion protein comprising an ST2 domain and an anti-HSA sdAb.

In one embodiment, the fusion protein provided herein comprises an ST2 domain, an anti-HSA sdAb, and optionally a peptide linker; wherein the C-terminus of the ST2 domain is connected to the N-terminus of the anti-HSA sdAb directly or via the peptide linker; or wherein the N-terminus of the ST2 domain is connected to the C-terminus of the anti-HSA sdAb directly or via the peptide linker.

In another embodiment, the fusion protein provided herein comprises an ST2 domain, an anti-HSA sdAb, and optionally a peptide linker; wherein the C-terminus of the ST2 domain is connected to the N-terminus of the anti-HSA sdAb directly or via the peptide linker.

In yet another embodiment, the fusion protein provided herein comprises an ST2 domain, an anti-HSA sdAb, and a peptide linker; wherein the C-terminus of the ST2 domain is connected to the N-terminus of the anti-HSA sdAb via the peptide linker.

In yet another embodiment, the fusion protein provided herein comprises an ST2 domain, an anti-HSA sdAb, and optionally a peptide linker; wherein the N-terminus of the ST2 domain is connected to the C-terminus of the anti-HSA sdAb directly or via the peptide linker.

In still another embodiment, the fusion protein provided herein comprises an ST2 domain, an anti-HSA sdAb, and a peptide linker; wherein the N-terminus of the ST2 domain is connected to the C-terminus of the anti-HSA sdAb via the peptide linker.

In still another embodiment, provided herein is a fusion protein comprising an IL-33 domain, an ST2 domain, and an anti-HSA sdAb.

In one embodiment, the fusion protein provided herein comprises an IL-33 domain, an ST2 domain, an anti-HSA sdAb, and optionally first and second peptide linkers; wherein the C-terminus of the ST2 domain is connected to the N-terminus of the anti-HSA sdAb directly or via the first peptide linker, and the C-terminus of the anti-HSA sdAb is connected to the N-terminus of the IL-33 domain directly or via the second peptide linker.

In another embodiment, the fusion protein provided herein comprises an IL-33 domain, an ST2 domain, an anti-HSA sdAb, and first and second peptide linkers; wherein the C-terminus of the ST2 domain is connected to the N-terminus of the anti-HSA sdAb via the first peptide linker, and the C-terminus of the anti-HSA sdAb is connected to the N-terminus of the IL-33 domain via the second peptide linker.

In yet another embodiment, the fusion protein provided herein comprises an IL-33 domain, an ST2 domain, an anti-HSA sdAb, and optionally first and second peptide linkers; wherein the C-terminus of the ST2 domain is connected to the N-terminus of the IL-33 domain directly or via the first peptide linker, and the C-terminus of the IL-33 domain is connected to the N-terminus of the anti-HSA sdAb directly or via the second peptide linker.

In yet another embodiment, the fusion protein provided herein comprises an IL-33 domain, an ST2 domain, an anti-HSA sdAb, and first and second peptide linkers; wherein the C-terminus of the ST2 domain is connected to the N-terminus of the IL-33 domain via the first peptide linker, and the C-terminus of the IL-33 domain is connected to the N-terminus of the anti-HSA sdAb via the second peptide linker.

In yet another embodiment, the fusion protein provided herein comprises an IL-33 domain, an ST2 domain, an anti-HSA sdAb, and optionally first and second peptide linkers; wherein the C-terminus of the anti-HSA sdAb is connected to the N-terminus of the IL-33 domain directly or via the first peptide linker, and the C-terminus of the IL-33 domain is connected to the N-terminus of the ST2 domain directly or via the second peptide linker.

In yet another embodiment, the fusion protein provided herein comprises an IL-33 domain, an ST2 domain, an anti-HSA sdAb, and first and second peptide linkers; wherein the C-terminus of the anti-HSA sdAb is connected to the N-terminus of the IL-33 domain via the first peptide linker, and the C-terminus of the IL-33 domain is connected to the N-terminus of the ST2 domain via the second peptide linker.

In yet another embodiment, the fusion protein provided herein comprises an IL-33 domain, an ST2 domain, an anti-HSA sdAb, and optionally first and second peptide linkers; wherein the C-terminus of the anti-HSA sdAb is connected to the N-terminus of the ST2 domain directly or via the first peptide linker, and the C-terminus of the ST2 domain is connected to the N-terminus of the IL-33 domain directly or via the second peptide linker.

In yet another embodiment, the fusion protein provided herein comprises an IL-33 domain, an ST2 domain, an anti-HSA sdAb, and first and second peptide linkers; wherein the C-terminus of the anti-HSA sdAb is connected to the N-terminus of the ST2 domain via the first peptide linker, and the C-terminus of the ST2 domain is connected to the N-terminus of the IL-33 domain via the second peptide linker.

In yet another embodiment, the fusion protein provided herein comprises an IL-33 domain, an ST2 domain, an anti-HSA sdAb, and optionally first and second peptide linkers; wherein the C-terminus of the IL-33 is connected to the N-terminus of the anti-HSA sdAb directly or via the first peptide linker, and the C-terminus of the anti-HSA sdAb is connected to the N-terminus of the ST2 domain directly or via the second peptide linker.

In yet another embodiment, the fusion protein provided herein comprises an IL-33 domain, an ST2 domain, an anti-HSA sdAb, and first and second peptide linkers; wherein the C-terminus of the IL-33 is connected to the N-terminus of the anti-HSA sdAb via the first peptide linker, and the C-terminus of the anti-HSA sdAb is connected to the N-terminus of the ST2 domain via the second peptide linker.

In yet another embodiment, the fusion protein provided herein comprises an IL-33 domain, an ST2 domain, an anti-HSA sdAb, and optionally first and second peptide linkers; wherein the C-terminus of the IL-33 is connected to the N-terminus of the ST2 domain directly or via the first peptide linker, and the C-terminus of the ST2 domain is connected to the N-terminus of the anti-HSA sdAb directly or via the second peptide linker.

In still another embodiment, the fusion protein provided herein comprises an IL-33 domain, an ST2 domain, an anti-HSA sdAb, and first and second peptide linkers; wherein the C-terminus of the IL-33 is connected to the N-terminus of the ST2 domain via the first peptide linker, and the C-terminus of the ST2 domain is connected to the N-terminus of the anti-HSA sdAb via the second peptide linker.

In one embodiment, the anti-HSA antibody is a V_{H}H sdAb that binds to an HSA. In certain embodiments, the anti-HSA antibody is V_{H}H sdAb that binds to an HSA specifically.

In certain embodiments, the anti-HSA V_{H}H sdAb binds to an HSA with a K_{d} ranging from about 10 pM to about 1,000 nM, from about 1 to about 500 nM, from about 1 to about 200 nM, or from about 1 to about 100 nM at a pH of about 7. In certain embodiments, the anti-HSA V_{H}H sdAb binds to an HSA with a K_{d} ranging from about 10 pM to about 1,000 nM at a pH of about 7. In certain embodiments, the anti-HSA V_{H}H sdAb binds to an HSA with a K_{d} ranging from about 1 to about 500 nM at a pH of about 7. In certain embodiments, the anti-HSA V_{H}H sdAb binds to an HSA with a K_{d} ranging from about 1 to about 200 nM at a pH of about 7. In certain embodiments, the anti-HSA V_{H}H sdAb binds to an HSA with a K_{d} ranging from about 1 to about 100 nM at a pH of about 7.

In one embodiment, the anti-HSA V_{H}H sdAb comprises (i) a heavy chain CDR1 of SEQ ID NO: 1, a heavy chain CDR2 of SEQ ID NO: 2, and a heavy chain CDR3 of SEQ ID NO: 3; or (ii) a heavy chain CDR1 of SEQ ID NO: 9, a heavy chain CDR2 of SEQ ID NO: 10, and a heavy chain CDR3 of SEQ ID NO: 11. In another embodiment, the anti-HSA V_{H}H sdAb comprises a heavy chain CDR1 of SEQ ID NO: 1, a heavy chain CDR2 of SEQ ID NO: 2, and a heavy chain CDR3 of SEQ ID NO: 3. In yet another embodiment, the anti-HSA V_{H}H sdAb comprises a heavy chain CDR1 of SEQ ID NO: 9, a heavy chain CDR2 of SEQ ID NO: 10, and a heavy chain CDR3 of SEQ ID NO: 11.

In one embodiment, the anti-HSA V_{H}H sdAb has the structure of FR1-CDR1-FR2-CDR2-FR3-CDR3-FR4, wherein:
CDR1, CDR2, and CDR3 are:
   (i) CDR1 of SEQ ID NO: 1, CDR2 of SEQ ID NO: 2, and CDR3 of SEQ ID NO: 3; or
   (ii) CDR1 of SEQ ID NO: 9, CDR2 of SEQ ID NO: 10, and CDR3 of SEQ ID NO: 11;
FR1 is an amino acid sequence of SEQ ID NO: 4 or 12;
FR2 is an amino acid sequence of SEQ ID NO: 5 or 13;
FR3 is an amino acid sequence of SEQ ID NO: 6; and
FR4 is an amino acid sequence of SEQ ID NO: 7 or 14.

In another embodiment, the anti-HSA V_{H}H sdAb has the structure of FR1-CDR1-FR2-CDR2-FR3-CDR3-FR4, wherein:
CDR1, CDR2, and CDR3 are:
   (i) CDR1 of SEQ ID NO: 1, CDR2 of SEQ ID NO: 2, and CDR3 of SEQ ID NO: 3; or
   (ii) CDR1 of SEQ ID NO: 9, CDR2 of SEQ ID NO: 10, and CDR3 of SEQ ID NO: 11;
FR1 is an amino acid sequence of SEQ ID NO: 4;
FR2 is an amino acid sequence of SEQ ID NO: 5;
FR3 is an amino acid sequence of SEQ ID NO: 6; and
FR4 is an amino acid sequence of SEQ ID NO: 7.

In yet another embodiment, the anti-HSA V_{H}H sdAb has the structure of FR1-CDR1-FR2-CDR2-FR3-CDR3-FR4, wherein:
CDR1, CDR2, and CDR3 are:
   (i) CDR1 of SEQ ID NO: 1, CDR2 of SEQ ID NO: 2, and CDR3 of SEQ ID NO: 3; or
   (ii) CDR1 of SEQ ID NO: 9, CDR2 of SEQ ID NO: 10, and CDR3 of SEQ ID NO: 11;
FR1 is an amino acid sequence of SEQ ID NO: 12;
FR2 is an amino acid sequence of SEQ ID NO: 13;
FR3 is an amino acid sequence of SEQ ID NO: 6; and
FR4 is an amino acid sequence of SEQ ID NO: 14.

In one embodiment, the anti-HSA V_{H}H sdAb has an amino acid sequence of SEQ ID NO: 8 or 15. In another embodiment, the anti-HSA V_{H}H sdAb has an amino acid sequence of SEQ ID NO: 8 or 15. In yet another embodiment, the anti-HSA V_{H}H sdAb has an amino acid sequence of SEQ ID NO: 8 or 15.

In certain embodiments, the anti-HSA V_{H}H sdAb is one disclosed in WO 2019/246004 A1 or WO 2020/172528 A1, the disclosure of each of which is incorporated herein by reference in its entirety.

In certain embodiments, the anti-HSA V_{H}H sdAb is a human antibody. In certain embodiments, the anti-HSA V_{H}H sdAb is a humanized antibody.

In one embodiment, provided herein is a fusion protein comprising an IL-33 domain or an ST2 domain, and an anti-HSA V_{H}H sdAb.

In another embodiment, provided herein is a fusion protein comprising an IL-33 domain and an anti-HSA V_{H}H sdAb.

In one embodiment, the fusion protein provided herein comprises an IL-33 domain, an anti-HSA V_{H}H sdAb, and optionally a peptide linker; wherein the C-terminus of the IL-33 domain is connected to the N-terminus of the anti-HSA V_{H}H sdAb directly or via the peptide linker; or wherein the N-terminus of the IL-33 domain is connected to the C-terminus of the anti-HSA V_{H}H sdAb directly or via the peptide linker.

In another embodiment, the fusion protein provided herein comprises an IL-33 domain, an anti-HSA V_{H}H sdAb, and optionally a peptide linker; wherein the C-terminus of the IL-33 domain is connected to the N-terminus of the anti-HSA V_{H}H sdAb directly or via the peptide linker.

In yet another embodiment, the fusion protein provided herein comprises an IL-33 domain, an anti-HSA V_{H}H sdAb, and a peptide linker; wherein the C-terminus of the IL-33 domain is connected to the N-terminus of the anti-HSA V_{H}H sdAb via the peptide linker.

In yet another embodiment, the fusion protein provided herein comprises an IL-33 domain, an anti-HSA V_{H}H sdAb, and optionally a peptide linker; wherein the N-terminus of the IL-33 domain is connected to the C-terminus of the anti-HSA V_{H}H sdAb directly or via the peptide linker.

In still another embodiment, the fusion protein provided herein comprises an IL-33 domain, an anti-HSA V_{H}H sdAb, and a peptide linker; wherein the N-terminus of the IL-33 domain is connected to the C-terminus of the anti-HSA V_{H}H sdAb via the peptide linker.

In yet another embodiment, provided herein is a fusion protein comprising an ST2 domain and an anti-HSA V_{H}H sdAb.

In one embodiment, the fusion protein provided herein comprises an ST2 domain, an anti-HSA V_{H}H sdAb, and optionally a peptide linker; wherein the C-terminus of the ST2 domain is connected to the N-terminus of the anti-HSA V_{H}H sdAb directly or via the peptide linker; or wherein the N-terminus of the ST2 domain is connected to the C-terminus of the anti-HSA V_{H}H sdAb directly or via the peptide linker.

In another embodiment, the fusion protein provided herein comprises an ST2 domain, an anti-HSA V_{H}H sdAb, and optionally a peptide linker; wherein the C-terminus of the ST2 domain is connected to the N-terminus of the anti-HSA V_{H}H sdAb directly or via the peptide linker.

In yet another embodiment, the fusion protein provided herein comprises an ST2 domain, an anti-HSA V_{H}H sdAb, and a peptide linker; wherein the C-terminus of the ST2 domain is connected to the N-terminus of the anti-HSA V_{H}H sdAb via the peptide linker.

In yet another embodiment, the fusion protein provided herein comprises an ST2 domain, an anti-HSA V_{H}H sdAb, and optionally a peptide linker; wherein the N-terminus of the ST2 domain is connected to the C-terminus of the anti-HSA V_{H}H sdAb directly or via the peptide linker.

In still another embodiment, the fusion protein provided herein comprises an ST2 domain, an anti-HSA V_{H}H sdAb, and a peptide linker; wherein the N-terminus of the ST2 domain is connected to the C-terminus of the anti-HSA V_{H}H sdAb via the peptide linker.

In still another embodiment, provided herein is a fusion protein comprising an IL-33 domain, an ST2 domain, and an anti-HSA V_{H}H sdAb.

In one embodiment, the fusion protein provided herein comprises an IL-33 domain, an ST2 domain, an anti-HSA V_{H}H sdAb, and optionally first and second peptide linkers; wherein the C-terminus of the ST2 domain is connected to the N-terminus of the anti-HSA V_{H}H sdAb directly or via the first peptide linker, and the C-terminus of the anti-HSA V_{H}H sdAb is connected to the N-terminus of the IL-33 domain directly or via the second peptide linker.

In another embodiment, the fusion protein provided herein comprises an IL-33 domain, an ST2 domain, an anti-HSA V_{H}H sdAb, and first and second peptide linkers; wherein the C-terminus of the ST2 domain is connected to the N-terminus of the anti-HSA V_{H}H sdAb via the first peptide linker, and the C-terminus of the anti-HSA V_{H}H sdAb is connected to the N-terminus of the IL-33 domain via the second peptide linker.

In yet another embodiment, the fusion protein provided herein comprises an IL-33 domain, an ST2 domain, an anti-HSA V_{H}H sdAb, and optionally first and second peptide linkers; wherein the C-terminus of the ST2 domain is connected to the N-terminus of the IL-33 domain directly or via the first peptide linker, and the C-terminus of the IL-33 domain is connected to the N-terminus of the anti-HSA V_{H}H sdAb directly or via the second peptide linker.

In yet another embodiment, the fusion protein provided herein comprises an IL-33 domain, an ST2 domain, an anti-HSA V_{H}H sdAb, and first and second peptide linkers; wherein the C-terminus of the ST2 domain is connected to the N-terminus of the IL-33 domain via the first peptide linker, and the C-terminus of the IL-33 domain is connected to the N-terminus of the anti-HSA V_{H}H sdAb via the second peptide linker.

In yet another embodiment, the fusion protein provided herein comprises an IL-33 domain, an ST2 domain, an anti-HSA V_{H}H sdAb, and optionally first and second peptide linkers; wherein the C-terminus of the anti-HSA V_{H}H sdAb is connected to the N-terminus of the IL-33 domain directly or via the first peptide linker, and the C-terminus of the IL-33 domain is connected to the N-terminus of the ST2 domain directly or via the second peptide linker.

In yet another embodiment, the fusion protein provided herein comprises an IL-33 domain, an ST2 domain, an anti-HSA V_{H}H sdAb, and first and second peptide linkers; wherein the C-terminus of the anti-HSA V_{H}H sdAb is connected to the N-terminus of the IL-33 domain via the first peptide linker, and the C-terminus of the IL-33 domain is connected to the N-terminus of the ST2 domain via the second peptide linker.

In yet another embodiment, the fusion protein provided herein comprises an IL-33 domain, an ST2 domain, an anti-HSA V_{H}H sdAb, and optionally first and second peptide linkers; wherein the C-terminus of the anti-HSA V_{H}H sdAb is connected to the N-terminus of the ST2 domain directly or via the first peptide linker, and the C-terminus of the ST2 domain is connected to the N-terminus of the IL-33 domain directly or via the second peptide linker.

In yet another embodiment, the fusion protein provided herein comprises an IL-33 domain, an ST2 domain, an anti-HSA V_{H}H sdAb, and first and second peptide linkers; wherein the C-terminus of the anti-HSA V_{H}H sdAb is connected to the N-terminus of the ST2 domain via the first peptide linker, and the C-terminus of the ST2 domain is connected to the N-terminus of the IL-33 domain via the second peptide linker.

In yet another embodiment, the fusion protein provided herein comprises an IL-33 domain, an ST2 domain, an anti-HSA V_{H}H sdAb, and optionally first and second peptide linkers; wherein the C-terminus of the IL-33 is connected to the N-terminus of the anti-HSA V_{H}H sdAb directly or via the first peptide linker, and the C-terminus of the anti-HSA V_{H}H sdAb is connected to the N-terminus of the ST2 domain directly or via the second peptide linker.

In yet another embodiment, the fusion protein provided herein comprises an IL-33 domain, an ST2 domain, an anti-HSA V_{H}H sdAb, and first and second peptide linkers; wherein the C-terminus of the IL-33 is connected to the N-terminus of the anti-HSA V_{H}H sdAb via the first peptide linker, and the C-terminus of the anti-HSA V_{H}H sdAb is connected to the N-terminus of the ST2 domain via the second peptide linker.

In yet another embodiment, the fusion protein provided herein comprises an IL-33 domain, an ST2 domain, an anti-HSA V_{H}H sdAb, and optionally first and second peptide linkers; wherein the C-terminus of the IL-33 is connected to the N-terminus of the ST2 domain directly or via the first peptide linker, and the C-terminus of the ST2 domain is connected to the N-terminus of the anti-HSA V_{H}H sdAb directly or via the second peptide linker.

In still another embodiment, the fusion protein provided herein comprises an IL-33 domain, an ST2 domain, an anti-HSA V_{H}H single domain antibody, and first and second peptide linkers; wherein the C-terminus of the IL-33 is connected to the N-terminus of the ST2 domain via the first peptide linker, and the C-terminus of the ST2 domain is connected to the N-terminus of the anti-HSA V_{H}H single domain antibody via the second peptide linker.

In one embodiment, provided herein is a fusion protein comprising an IL-33 domain or an ST2 domain, and a Fc domain.

In another embodiment, provided herein is a fusion protein comprising an IL-33 domain and an Fc domain.

In one embodiment, the fusion protein provided herein comprises an IL-33 domain, an Fc domain comprising first and second peptide chains, and optionally a peptide linker; wherein the C-terminus of the IL-33 domain is connected to the N-terminus of the first peptide chain of the Fc domain directly or via the peptide linker, or the C-terminus of the first peptide chain of the Fc domain is connected to the N-terminus of the IL-33 domain directly or via the peptide linker.

In another embodiment, the fusion protein provided herein comprises an IL-33 domain, an Fc domain comprising first and second peptide chains, and optionally a peptide linker; wherein the C-terminus of the IL-33 domain is connected to the N-terminus of the first peptide chain of the Fc domain directly or via the peptide linker.

In yet another embodiment, the fusion protein provided herein comprises an IL-33 domain, an Fc domain comprising first and second peptide chains, and a peptide linker; wherein the C-terminus of the IL-33 domain is connected to the N-terminus of the first peptide chain of the Fc domain via the peptide linker.

In yet another embodiment, the fusion protein provided herein comprises an IL-33 domain, an Fc domain comprising first and second peptide chains, and optionally a peptide linker; wherein the C-terminus of the first peptide chain of the Fc domain is connected to the N-terminus of the IL-33 domain directly or via the peptide linker.

In still another embodiment, the fusion protein provided herein comprises an IL-33 domain, an Fc domain comprising first and second peptide chains, and a peptide linker; wherein the C-terminus of the first peptide chain of the Fc domain is connected to the N-terminus of the IL-33 domain via the peptide linker.

In yet another embodiment, provided herein is a fusion protein comprising an ST2 domain and an Fc domain.

In one embodiment, the fusion protein provided herein comprises an ST2 domain, an Fc domain comprising first and second peptide chains, and optionally a peptide linker; wherein the C-terminus of the ST2 domain is connected to the N-terminus of the first peptide chain of the Fc domain directly or via the peptide linker, or the C-terminus of the first peptide chain of the Fc domain is connected to the N-terminus of the ST2 domain directly or via the peptide linker.

In another embodiment, the fusion protein provided herein comprises an ST2 domain, an Fc domain comprising first and second peptide chains, and optionally a peptide linker; wherein the C-terminus of the ST2 domain is connected to the N-terminus of the first peptide chain of the Fc domain directly or via the peptide linker.

In yet another embodiment, the fusion protein provided herein comprises an ST2 domain, an Fc domain comprising first and second peptide chains, and a peptide linker; wherein the C-terminus of the ST2 domain is connected to the N-terminus of the first peptide chain of the Fc domain via the peptide linker.

In yet another embodiment, the fusion protein provided herein comprises an ST2 domain, an Fc domain comprising first and second peptide chains, and optionally a peptide linker; wherein the C-terminus of the first peptide chain of the Fc domain is connected to the N-terminus of the ST2 domain directly or via the peptide linker.

In still another embodiment, the fusion protein provided herein comprises an ST2 domain, an Fc domain comprising first and second peptide chains, and a peptide linker; wherein the C-terminus of the first peptide chain of the Fc domain is connected to the N-terminus of the ST2 domain via the peptide linker.

In yet another embodiment, provided herein is a fusion protein comprising two IL-33 domains and an Fc domain.

In one embodiment, the fusion protein provided herein comprises first and second IL-33 domains, an Fc domain comprising first and second peptide chains, and optionally first and second peptide linkers; wherein the C-terminus of the first IL-33 domain is connected to the N-terminus of the first peptide chain of the Fc domain directly or via the first peptide linker, and the C-terminus of the second IL-33 domain is connected to the N-terminus of the second peptide chain of the Fc domain directly or via the second peptide linker.

In another embodiment, the fusion protein provided herein comprises first and second IL-33 domains, and an Fc domain comprising first and second peptide chains; wherein the C-terminus of the first IL-33 domain is connected to the N-terminus of the first peptide chain of the Fc domain directly, and the C-terminus of the second IL-33 domain is connected to the N-terminus of the second peptide chain of the Fc domain directly.

In yet another embodiment, the fusion protein provided herein comprises first and second IL-33 domains, an Fc domain comprising first and second peptide chains, and first and second peptide linkers; wherein the C-terminus of the first IL-33 domain is connected to the N-terminus of the first peptide chain of the Fc domain via the first peptide linker, and the C-terminus of the second IL-33 domain is connected to the N-terminus of the second peptide chain of the Fc domain via the second peptide linker.

In yet another embodiment, the fusion protein provided herein comprises first and second IL-33 domains, an Fc domain comprising first and second peptide chains, and optionally first and second peptide linkers; wherein the N-terminus of the first IL-33 domain is connected to the C-terminus of the first peptide chain of the Fc domain directly or via the first peptide linker, and the C-terminus of the second IL-33 domain is connected to the N-terminus of the second peptide chain of the Fc domain directly or via the second peptide linker.

In yet another embodiment, the fusion protein provided herein comprises first and second IL-33 domains, an Fc domain comprising first and second peptide chains, and first and second peptide linkers; wherein the N-terminus of the first IL-33 domain is connected to the C-terminus of the first peptide chain of the Fc domain via the first peptide linker, and the C-terminus of the second IL-33 domain is connected to the N-terminus of the second peptide chain of the Fc domain via the second peptide linker.

In yet another embodiment, the fusion protein provided herein comprises first and second IL-33 domains, an Fc domain comprising first and second peptide chains, and optionally first and second peptide linkers; wherein the N-terminus of the first IL-33 domain is connected to the C-terminus of the first peptide chain of the Fc domain directly or via the first peptide linker, and the N-terminus of the second IL-33 domain is connected to the C-terminus of the second peptide chain of the Fc domain directly or via the second peptide linker.

In still another embodiment, the fusion protein provided herein comprises first and second IL-33 domains, an Fc domain comprising first and second peptide chains, and first and second peptide linkers; wherein the N-terminus of the first IL-33 domain is connected to the C-terminus of the first peptide chain of the Fc domain via the first peptide linker, and the N-terminus of the second IL-33 domain is connected to the C-terminus of the second peptide chain of the Fc domain via the second peptide linker.

In yet another embodiment, provided herein is a fusion protein comprising two ST2 domains and an Fc domain.

In one embodiment, the fusion protein provided herein comprises first and second ST2 domains, an Fc domain comprising first and second peptide chains, and optionally first and second peptide linkers; wherein the C-terminus of the first ST2 domain is connected to the N-terminus of the first peptide chain of the Fc domain directly or via the first peptide linker, and the C-terminus of the second ST2 domain is connected to the N-terminus of the second peptide chain of the Fc domain directly or via the second peptide linker.

In another embodiment, the fusion protein provided herein comprises first and second ST2 domains, and an Fc domain comprising first and second peptide chains; wherein the C-terminus of the first ST2 domain is connected to the N-terminus of the first peptide chain of the Fc domain directly, and the C-terminus of the second ST2 domain is connected to the N-terminus of the second peptide chain of the Fc domain directly.

In yet another embodiment, the fusion protein provided herein comprises first and second ST2 domains, an Fc domain comprising first and second peptide chains, and first and second peptide linkers; wherein the C-terminus of the first ST2 domain is connected to the N-terminus of the first peptide chain of the Fc domain via the first peptide linker, and the C-terminus of the second ST2 domain is connected to the N-terminus of the second peptide chain of the Fc domain via the second peptide linker.

In yet another embodiment, the fusion protein provided herein comprises first and second ST2 domains, an Fc domain comprising first and second peptide chains, and optionally first and second peptide linkers; wherein the N-terminus of the first ST2 domain is connected to the C-terminus of the first peptide chain of the Fc domain directly or via the first peptide linker, and the C-terminus of the second ST2 domain is connected to the N-terminus of the second peptide chain of the Fc domain directly or via the second peptide linker.

In yet another embodiment, the fusion protein provided herein comprises first and second ST2 domains, an Fc domain comprising first and second peptide chains, and first and second peptide linkers; wherein the N-terminus of the first ST2 domain is connected to the C-terminus of the first peptide chain of the Fc domain via the first peptide linker, and the C-terminus of the second ST2 domain is connected to the N-terminus of the second peptide chain of the Fc domain via the second peptide linker.

In yet another embodiment, the fusion protein provided herein comprises first and second ST2 domains, an Fc domain comprising first and second peptide chains, and optionally first and second peptide linkers; wherein the N-terminus of the first ST2 domain is connected to the C-terminus of the first peptide chain of the Fc domain directly or via the first peptide linker, and the N-terminus of the second ST2 domain is connected to the C-terminus of the second peptide chain of the Fc domain directly or via the second peptide linker.

In still another embodiment, the fusion protein provided herein comprises first and second ST2 domains, an Fc domain comprising first and second peptide chains, and first and second peptide linkers; wherein the N-terminus of the first ST2 domain is connected to the C-terminus of the first peptide chain of the Fc domain via the first peptide linker, and the N-terminus of the second ST2 domain is connected to the C-terminus of the second peptide chain of the Fc domain via the second peptide linker.

In yet another embodiment, provided herein is a fusion protein comprising an IL-33 domain, an ST2 domain, and an Fc domain.

In one embodiment, the fusion protein provided herein comprises an IL-33 domain, an ST2 domain, an Fc domain comprising first and second peptide chains, and optionally first and second peptide linkers; wherein the C-terminus of the first peptide chain of the Fc domain is connected to the N-terminus of the ST2 domain directly or via the first peptide linker, and the C-terminus of the ST2 domain is connected to the N-terminus of the IL-33 domain directly or via the second peptide linker.

In another embodiment, the fusion protein provided herein comprises an IL-33 domain, an ST2 domain, an Fc domain comprising first and second peptide chains, and first and second peptide linkers; wherein the C-terminus of the first peptide chain of the Fc domain is connected to the N-terminus of the ST2 domain via the first peptide linker, and the C-terminus of the ST2 domain is connected to the N-terminus of the IL-33 domain via the second peptide linker.

In yet another embodiment, the fusion protein provided herein comprises an IL-33 domain, an ST2 domain, an Fc domain comprising first and second peptide chains, and optionally first and second peptide linkers; wherein the C-terminus of the first peptide chain of the Fc domain is connected to the N-terminus of the IL-33 domain directly or via the first peptide linker, and the C-terminus of the second peptide chain of the Fc domain is connected to the N-terminus of the ST2 domain directly or via the second peptide linker.

In still another embodiment, the fusion protein provided herein comprises an IL-33 domain, an ST2 domain, an Fc domain comprising first and second peptide chains, and first and second peptide linkers; wherein the C-terminus of the first peptide chain of the Fc domain is connected to the N-terminus of the IL-33 domain via the first peptide linker, and the C-terminus of the second peptide chain of the Fc domain is connected to the N-terminus of the ST2 domain via the second peptide linker.

In still another embodiment, provided herein is a fusion protein comprising two IL-33 domains, two ST2 domains, and an Fc domain.

In one embodiment, the fusion protein provided herein comprises first and second IL-33 domains, first and second ST2 domains, an Fc domain comprising first and second peptide chains, and optionally first, second, third, and fourth peptide linkers; wherein the C-terminus of the first peptide chain of the Fc domain is connected to the N-terminus of the first ST2 domain directly or via the first peptide linker, and the C-terminus of the N-terminus of the first ST2 domain is connected to the N-terminus of the first IL-33 domain directly or via the second peptide linker; and wherein the C-terminus of the second peptide chain of the Fc domain is connected to the N-terminus of the second ST2 domain directly or via the third peptide linker, and the C-terminus of the N-terminus of the second ST2 domain is connected to the N-terminus of the second IL-33 domain directly or via the fourth peptide linker.

In another embodiment, the fusion protein provided herein comprises first and second IL-33 domains, first and second ST2 domains, an Fc domain comprising first and second peptide chains, and first, second, third, and fourth peptide linkers; wherein the C-terminus of the first peptide chain of the Fc domain is connected to the N-terminus of the first ST2 domain via the first peptide linker, and the C-terminus of the N-terminus of the first ST2 domain is connected to the N-terminus of the first IL-33 domain via the second peptide linker; and wherein the C-terminus of the second peptide chain of the Fc domain is connected to the N-terminus of the second ST2 domain via the third peptide linker, and the C-terminus of the N-terminus of the second ST2 domain is connected to the N-terminus of the second IL-33 domain via the fourth peptide linker.

In one embodiment, the Fc domain is a hIgG1 Fc domain or a mutein thereof, or a fragment thereof. In another embodiment, the Fc domain is a hIgG1 Fc having an amino acid substitution of N297A. In yet another embodiment, the Fc domain is a hIgG2 Fc domain or a mutein thereof, or a fragment thereof. In still another embodiment, the Fc domain is a hIgG4 Fc domain or a mutein thereof, or a fragment thereof.

In one embodiment, the Fc domain as a half-life-extension domain comprises an amino acid sequence of SEQ ID NO: 55, 56, 57, 58, 59, 60, 61, 62, 63, 64, 65, or 66. Thus, in one embodiment, the Fc domain comprises an amino acid sequence of SEQ ID NO: 55. In another embodiment, the Fc domain comprises an amino acid sequence of SEQ ID NO: 56. In yet another embodiment, the Fc domain comprises an amino acid sequence of SEQ ID NO: 57. In yet another embodiment, the Fc domain comprises an amino acid sequence of SEQ ID NO: 58. In yet another embodiment, the Fc domain comprises an amino acid sequence of SEQ ID NO: 59. In yet another embodiment, the Fc domain comprises an amino acid sequence of SEQ ID NO: 60. In yet another embodiment, the Fc domain comprises an amino acid sequence of SEQ ID NO: 61. In yet another embodiment, the Fc domain comprises an amino acid sequence of SEQ ID NO: 62. In yet another embodiment, the Fc domain comprises an amino acid sequence of SEQ ID NO: 63. In yet another embodiment, the Fc domain comprises an amino acid sequence of SEQ ID NO: 64. In yet another embodiment, the Fc domain comprises an amino acid sequence of SEQ ID NO: 65. In still another embodiment, the Fc domain comprises an amino acid sequence of SEQ ID NO: 66.

In one embodiment, the Fc domain comprises a pair of peptide chains in a knobs-in-holes (KIH) configuration. Thus, in one embodiment, the Fc domain comprises amino acid sequences of SEQ ID NO: 58 and 59, 60 and 61, 62 and 63, or 65 and 66 as a pair of peptide chains in a knobs-in-holes configuration. In another embodiment, the Fc domain comprises amino acid sequences of SEQ ID NO: 58 and 59 as a pair of peptide chains in a knobs-in-holes configuration. In yet another embodiment, the Fc domain comprises amino acid sequences of SEQ ID NO: 60 and 61 as a pair of peptide chains in a knobs-in-holes configuration. In yet another embodiment, the Fc domain comprises amino acid sequences of SEQ ID NO: 62 and 63 as a pair of peptide chains in a knobs-in-holes configuration. In still another embodiment, the Fc domain comprises amino acid sequences of SEQ ID NO: 65 and 66 as a pair of peptide chains in a knobs-in-holes configuration.

In one embodiment, provided herein is a fusion protein comprising an IL-33 domain, an ST2 domain, a PD-L1 binding domain, and an Fc domain.

In another embodiment, provided herein is a fusion protein comprising an IL-33 domain, an ST2 domain, two PD-L1 binding domains, and an Fc domain.

In one embodiment, the fusion protein provided herein comprises an IL-33 domain, an ST2 domain, first and second PD-L1 binding domains, an Fc domain comprising a first and second peptide chains, and optionally first and second peptide linkers; wherein a C-terminus of the first PD-L1 binding domain is connected to the N-terminus of the first peptide chain of the Fc domain directly or via a peptide linker, a C-terminus of the second PD-L1 binding domain is connected to the N-terminus of the second peptide chain of the Fc domain directly or via a peptide linker, the N-terminus of the ST2 domain is connected to the C-terminus of the first peptide chain of the Fc domain directly or via the first peptide linker, and the N-terminus of the IL-33 domain is connected to the C-terminus of the ST2 domain directly or via the second peptide linker.

In another embodiment, the fusion protein provided herein comprises an IL-33 domain, an ST2 domain, first and second PD-L1 binding domains, an Fc domain comprising a first and second peptide chains, and first and second peptide linkers; wherein a C-terminus of the first PD-L1 binding domain is connected to the N-terminus of the first peptide chain of the Fc domain directly or via a peptide linker, a C-terminus of the second PD-L1 binding domain is connected to the N-terminus of the second peptide chain of the Fc domain directly or via a peptide linker, the N-terminus of the ST2 domain is connected to the C-terminus of the first peptide chain of the Fc domain via the first peptide linker, and the N-terminus of the IL-33 domain is connected to the C-terminus of the ST2 domain via the second peptide linker.

In yet another embodiment, the fusion protein provided herein comprises an IL-33 domain, an ST2 domain, first and second PD-L1 binding domains, an Fc domain comprising a first and second peptide chains, and optionally first and second peptide linkers; wherein a C-terminus of the first PD-L1 binding domain is connected to the N-terminus of the first peptide chain of the Fc domain directly or via a peptide linker, a C-terminus of the second PD-L1 binding domain is connected to the N-terminus of the second peptide chain of the Fc domain directly or via a peptide linker, the N-terminus of the IL-33 domain is connected to the C-terminus of the first peptide chain of the Fc domain directly or via the first peptide linker, and the N-terminus of the ST2 domain is connected to the C-terminus of the second peptide chain of the Fc domain directly or via the second peptide linker.

In still another embodiment, the fusion protein provided herein comprises an IL-33 domain, an ST2 domain, first and second PD-L1 binding domains, an Fc domain comprising a first and second peptide chains, and first and second peptide linkers; wherein a C-terminus of the first PD-L1 binding domain is connected to the N-terminus of the first peptide chain of the Fc domain directly or via a peptide linker, a C-terminus of the second PD-L1 binding domain is connected to the N-terminus of the second peptide chain of the Fc domain directly or via a peptide linker, the N-terminus of the IL-33 domain is connected to the C-terminus of the first peptide chain of the Fc domain via the first peptide linker, and the N-terminus of the ST2 domain is connected to the C-terminus of the second peptide chain of the Fc domain via the second peptide linker.

In yet another embodiment, provided herein is a fusion protein comprising two IL-33 domains, two ST2 domains, two PD-L1 binding domains, and an Fc domain.

In one embodiment, the fusion protein provided herein comprises first and second IL-33 domains, first and second ST2 domains, first and second PD-L1 binding domains, an Fc domain comprising a first and second peptide chains, and optionally first, second, third, and fourth peptide linkers; wherein a C-terminus of the first PD-L1 binding domain is connected to the N-terminus of the first peptide chain of the Fc domain directly or via a peptide linker, a C-terminus of the second PD-L1 binding domain is connected to the N-terminus of the second peptide chain of the Fc domain directly or via a peptide linker; wherein the N-terminus of the first ST2 domain is connected to the C-terminus of the first peptide chain of the Fc domain directly or via the first peptide linker, and the N-terminus of the first IL-33 domain is connected to the C-terminus of the first ST2 domain directly or via the second peptide linker; and wherein the N-terminus of the second ST2 domain is connected to the C-terminus of the second peptide chain of the Fc domain directly or via the third peptide linker, and the N-terminus of the second IL-33 domain is connected to the C-terminus of the second ST2 domain directly or via the fourth peptide linker.

In another embodiment, the fusion protein provided herein comprises first and second IL-33 domains, first and second ST2 domains, first and second PD-L1 binding domains, an Fc domain comprising a first and second peptide chains, and first, second, third, and fourth peptide linkers; wherein a C-terminus of the first PD-L1 binding domain is connected to the N-terminus of the first peptide chain of the Fc domain directly or via a peptide linker, a C-terminus of the second PD-L1 binding domain is connected to the N-terminus of the second peptide chain of the Fc domain directly or via a peptide linker; wherein the N-terminus of the first ST2 domain is connected to the C-terminus of the first peptide chain of the Fc domain via the first peptide linker, and the N-terminus of the first IL-33 domain is connected to the C-terminus of the first ST2 domain via the second peptide linker; and wherein the N-terminus of the second ST2 domain is connected to the C-terminus of the second peptide chain of the Fc domain via the third peptide linker, and the N-terminus of the second IL-33 domain is connected to the C-terminus of the second ST2 domain via the fourth peptide linker.

In one embodiment, the PD-L1 binding domain is a single-chain variable fragment (scFv), Fab, Fab′, F (ab)₂, F(ab′)₂. Fv, diabody, triabody, tetrabody, minibody, or a V_{H}H single domain antibody.

In one embodiment, the PD-L1 binding domain is an scFv. In another embodiment, the PD-L1 binding domain is an scFv comprising a light chain (V_{L}), a heavy chain (V_{H}), and optionally a peptide linker, wherein the C-terminal of the light chain is connected to the N-terminal of the heavy chain directly or via the peptide linker, or wherein the N-terminal of the light chain is connected to the C-terminal of the heavy chain directly or via the peptide linker. In yet another embodiment, the PD-L1 binding domain is an scFv comprising a light chain (V_{L}), a heavy chain (V_{H}), and a peptide linker, wherein the C-terminal of the light chain is connected to the N-terminal of the heavy chain via the peptide linker, or wherein the N-terminal of the light chain is connected to the C-terminal of the heavy chain via the peptide linker. In yet another embodiment, the PD-L1 binding domain is an scFv comprising a light chain (V_{L}), a heavy chain (V_{H}), and a peptide linker, wherein the C-terminal of the light chain is connected to the N-terminal of the heavy chain via the peptide linker. In still another embodiment, the PD-L1 binding domain is an scFv comprising a light chain (V_{L}), a heavy chain (V_{H}), and a peptide linker, wherein the N-terminal of the light chain is connected to the C-terminal of the heavy chain via the peptide linker.

In one embodiment, the PD-L1 binding domain is a Fab. In another embodiment, the PD-L1 binding domain is a Fab′. In yet another embodiment, the PD-L1 binding domain is a F(ab)₂. In yet another embodiment, the PD-L1 binding domain is a F(ab′)₂. In yet another embodiment, the PD-L1 binding domain is a Fv. In yet another embodiment, the PD-L1 binding domain is a diabody. In yet another embodiment, the PD-L1 binding domain is a triabody. In yet another embodiment, the PD-L1 binding domain is a tetrabody. In yet another embodiment, the PD-L1 binding domain is a minibody. In yet another embodiment, the PD-L1 binding domain is a single domain antibody. In still another embodiment, the PD-L1 binding domain is a V_{H}H single domain antibody.

In one embodiment, the PD-L1 binding domain and Fc domain in a fusion protein provided herein are parts of an intact anti-PD-L1 antibody comprising two light chains and two heavy chains.

Thus, in one embodiment, provided herein is a fusion protein comprising an IL-33 domain, an ST2 domain, and an intact anti-PD-L1 antibody comprising two light chains and two heavy chains.

In one embodiment, the fusion protein provided herein comprises an IL-33 domain, an ST2 domain, an intact anti-PD-L1 antibody comprising first and second light chains and first and second heavy chains, and optionally first and second peptide linkers; wherein the N-terminus of the ST2 domain is connected to the C-terminus of the first heavy chain of the intact anti-PD-L1 antibody directly or via the first peptide linker, and the N-terminus of the IL-33 domain is connected to the C-terminus of the ST2 domain directly or via the second peptide linker.

In another embodiment, the fusion protein provided herein comprises an IL-33 domain, an ST2 domain, an intact anti-PD-L1 antibody comprising first and second light chains and first and second heavy chains, and first and second peptide linkers; wherein the N-terminus of the ST2 domain is connected to the C-terminus of the first heavy chain of the intact anti-PD-L1 antibody via the first peptide linker, and the N-terminus of the IL-33 domain is connected to the C-terminus of the ST2 domain via the second peptide linker.

In yet another embodiment, the fusion protein provided herein comprises an IL-33 domain, an ST2 domain, an intact anti-PD-L1 antibody comprising first and second light chains and first and second heavy chains, and optionally first and second peptide linkers; wherein the N-terminus of the ST2 domain is connected to the C-terminus of the first heavy chain of the intact anti-PD-L1 antibody directly or via the first peptide linker, and the N-terminus of the IL-33 domain is connected to the C-terminus of the second heavy chain of the intact anti-PD-L1 antibody directly or via the second peptide linker.

In still another embodiment, the fusion protein provided herein comprises an IL-33 domain, an ST2 domain, an intact anti-PD-L1 antibody comprising first and second light chains and first and second heavy chains, and first and second peptide linkers; wherein the N-terminus of the ST2 domain is connected to the C-terminus of the first heavy chain of the intact anti-PD-L1 antibody via the first peptide linker, and the N-terminus of the IL-33 domain is connected to the C-terminus of the second heavy chain of the intact anti-PD-L1 antibody via the second peptide linker.

In another embodiment, provided herein is a fusion protein comprising two IL-33 domains, two ST2 domains, and an intact anti-PD-L1 antibody comprising two light chains and two heavy chains.

In one embodiment, the fusion protein provided herein comprises first and second IL-33 domains, first and second ST2 domains, an intact anti-PD-L1 antibody comprising first and second light chains and first and second heavy chains, and optionally first, second, third, and fourth peptide linkers; wherein the N-terminus of the first ST2 domain is connected to the C-terminus of the first heavy chain of the intact anti-PD-L1 antibody directly or via the first peptide linker, and the N-terminus of the first IL-33 domain is connected to the C-terminus of the first ST2 domain directly or via the second peptide linker; and wherein the N-terminus of the second ST2 domain is connected to the C-terminus of the second heavy chain of the intact anti-PD-L1 antibody directly or via the third peptide linker, and the N-terminus of the second IL-33 domain is connected to the C-terminus of the second ST2 domain directly or via the fourth peptide linker.

In another embodiment, the fusion protein provided herein comprises first and second IL-33 domains, first and second ST2 domains, an intact anti-PD-L1 antibody comprising first and second light chains and first and second heavy chains, and optionally first, second, third, and fourth peptide linkers; wherein the N-terminus of the first ST2 domain is connected to the C-terminus of the first heavy chain of the intact anti-PD-L1 antibody via the first peptide linker, and the N-terminus of the first IL-33 domain is connected to the C-terminus of the first ST2 domain via the second peptide linker; and wherein the N-terminus of the second ST2 domain is connected to the C-terminus of the second heavy chain of the intact anti-PD-L1 antibody via the third peptide linker, and the N-terminus of the second IL-33 domain is connected to the C-terminus of the second ST2 domain via the fourth peptide linker.

In one embodiment, the intact PD-L1 antibody comprises:
(i) a light chain complementarity determining region 1 (CDRL1) of SEQ ID NO: 67, a light chain complementarity determining region 2 (CDRL2) of SEQ ID NO: 68, a light chain complementarity determining region 3 (CDRL3) of SEQ ID NO: 69, a heavy chain complementarity determining region 1 (CDRH1) of SEQ ID NO: 70, a heavy chain complementarity determining region 2 (CDRH2) of SEQ ID NO: 71, and a heavy chain complementarity determining region 3 (CDRH3) of SEQ ID NO: 72;
(ii) a CDRL1 of SEQ ID NO: 77, a CDRL2 of SEQ ID NO: 78, a CDRL3 of SEQ ID NO: 79, a CDRH1 of SEQ ID NO: 80, a CDRH2 of SEQ ID NO: 81, and a CDRH3 of SEQ ID NO: 82;
(iii) a CDRL1 of SEQ ID NO: 87, a CDRL2 of SEQ ID NO: 88, a CDRL3 of SEQ ID NO: 89, a CDRH1 of SEQ ID NO: 90, a CDRH2 of SEQ ID NO: 91, and a CDRH3 of SEQ ID NO: 92; or
(iv) a CDRL1 of SEQ ID NO: 97, a CDRL2 of SEQ ID NO: 98, a CDRL3 of SEQ ID NO: 99, a CDRH1 of SEQ ID NO: 100, a CDRH2 of SEQ ID NO: 101, and a CDRH3 of SEQ ID NO: 102.

In one embodiment, the intact PD-L1 antibody comprises a CDRL1 of SEQ ID NO: 67, a CDRL2 of SEQ ID NO: 68, a CDRL3 of SEQ ID NO: 69, a CDRH1 of SEQ ID NO: 70, a CDRH2 of SEQ ID NO: 71, and a CDRH3 of SEQ ID NO: 72. In another embodiment, the intact PD-L1 antibody comprises a CDRL1 of SEQ ID NO: 77, a CDRL2 of SEQ ID NO: 78, a CDRL3 of SEQ ID NO: 79, a CDRH1 of SEQ ID NO: 80, a CDRH2 of SEQ ID NO: 81, and a CDRH3 of SEQ ID NO: 82. In yet another embodiment, the intact PD-L1 antibody comprises a CDRL1 of SEQ ID NO: 87, a CDRL2 of SEQ ID NO: 88, a CDRL3 of SEQ ID NO: 89, a CDRH1 of SEQ ID NO: 90, a CDRH2 of SEQ ID NO: 91, and a CDRH3 of SEQ ID NO: 92. In still another embodiment, the intact PD-L1 antibody comprises a CDRL1 of SEQ ID NO: 97, a CDRL2 of SEQ ID NO: 98, a CDRL3 of SEQ ID NO: 99, a CDRH1 of SEQ ID NO: 100, a CDRH2 of SEQ ID NO: 101, and a CDRH3 of SEQ ID NO: 102.

In one embodiment, the intact PD-L1 antibody comprises:
(i) a light chain variable region of SEQ ID NO: 73 and a heavy chain variable region of SEQ ID NO: 74;
(ii) a light chain variable region of SEQ ID NO: 83 and a heavy chain variable region of SEQ ID NO: 84;
(iii) a light chain variable region of SEQ ID NO: 93 and a heavy chain variable region of SEQ ID NO: 94; or
(iv) a light chain variable region of SEQ ID NO: 103 and a heavy chain variable region of SEQ ID NO: 104.

In one embodiment, the intact PD-L1 antibody comprises a light chain variable region of SEQ ID NO: 73 and a heavy chain variable region of SEQ ID NO: 74. In another embodiment, the intact PD-L1 antibody comprises a light chain variable region of SEQ ID NO: 83 and a heavy chain variable region of SEQ ID NO: 84. In yet another embodiment, the intact PD-L1 antibody comprises a light chain variable region of SEQ ID NO: 93 and a heavy chain variable region of SEQ ID NO: 94. In still another embodiment, the intact PD-L1 antibody comprises a light chain variable region of SEQ ID NO: 103 and a heavy chain variable region of SEQ ID NO: 104.

In one embodiment, the intact PD-L1 antibody comprises:
(i) a light chain of SEQ ID NO: 75 and a heavy chain of SEQ ID NO: 76;
(ii) a light chain of SEQ ID NO: 85 and a heavy chain of SEQ ID NO: 86;
(iii) a light chain of SEQ ID NO: 95 and a heavy chain of SEQ ID NO: 96;
(iv) a light chain of SEQ ID NO: 105 and a heavy chain of SEQ ID NO: 106;
(v) a light chain of SEQ ID NO: 107 and a heavy chain of SEQ ID NO: 108;
(vi) a light chain of SEQ ID NO: 109 and a heavy chain of SEQ ID NO: 110; or
(vii) a light chain of SEQ ID NO: 111 and a heavy chain of SEQ ID NO: 112.

In one embodiment, the intact PD-L1 antibody comprises a light chain of SEQ ID NO: 75 and a heavy chain of SEQ ID NO: 76. In another embodiment, the intact PD-L1 antibody comprises a light chain of SEQ ID NO: 85 and a heavy chain of SEQ ID NO: 86. In yet another embodiment, the intact PD-L1 antibody comprises a light chain of SEQ ID NO: 95 and a heavy chain of SEQ ID NO: 96. In yet another embodiment, the intact PD-L1 antibody comprises a light chain of SEQ ID NO: 105 and a heavy chain of SEQ ID NO: 106. In yet another embodiment, the intact PD-L1 antibody comprises a light chain of SEQ ID NO: 107 and a heavy chain of SEQ ID NO: 108. In yet another embodiment, the intact PD-L1 antibody comprises a light chain of SEQ ID NO: 109 and a heavy chain of SEQ ID NO: 110. In still another embodiment, the intact PD-L1 antibody comprises a light chain of SEQ ID NO: 111 and a heavy chain of SEQ ID NO: 112.

In one embodiment, the intact PD-L1 antibody is an IgA, IgD, IgE, IgG, or IgM antibody. In another embodiment, the intact PD-L1 antibody is an IgA antibody. In yet another embodiment, the intact PD-L1 antibody is an IgD antibody. In yet another embodiment, the intact PD-L1 antibody is an IgE antibody. In yet another embodiment, the intact PD-L1 antibody is an IgG antibody. In still another embodiment, the intact PD-L1 antibody is an IgM antibody.

In one embodiment, the intact PD-L1 antibody is an IgA1, IgA2, IgG1, IgG2, IgG3, or IgG4 antibody. In another embodiment, the intact PD-L1 antibody is an IgA1 or IgA2. In yet another embodiment, the intact PD-L1 antibody is an IgA1. In yet another embodiment, the intact PD-L1 antibody is an IgA2. In yet another embodiment, the intact PD-L1 antibody is an IgG1, IgG2, IgG3, or IgG4 antibody. In yet another embodiment, the intact PD-L1 antibody is an IgG1 antibody. In yet another embodiment, the intact PD-L1 antibody is an IgG2 antibody. In yet another embodiment, the intact PD-L1 antibody is an IgG3 antibody. In still another embodiment, the intact PD-L1 antibody is an IgG4 antibody.

In one embodiment, the light chain of the intact PD-L1 antibody is a kappa or lambda chain. In another embodiment, the light chain of the intact PD-L1 antibody is a kappa chain. In yet another embodiment, the light chain of the intact PD-L1 antibody is a lambda chain.

In one embodiment, the intact PD-L1 antibody is a human antibody. In another embodiment, the intact PD-L1 antibody is a humanized antibody.

In one embodiment, the intact PD-L1 antibody is an antibody with a “knobs-into-holes” mutation. In another embodiment, the intact PD-L1 antibody is a human IgG1 antibody with an N297A mutation. In yet another embodiment, the intact PD-L1 antibody is a human IgG1 antibody with a “knobs-into-holes” mutation. In still another embodiment, the intact PD-L1 antibody is a human IgG4 antibody with a “knobs-into-holes” mutation.

In one embodiment, the PD-L1 binding domain is a single domain antibody. In another embodiment, the PD-L1 binding domain is a V_{H}H sdAb.

Thus, in one embodiment, provided herein is a fusion protein comprising an IL-33 domain, an ST2 domain, an anti-PD-L1 V_{H}H sdAb, and an Fc domain.

In one embodiment, the fusion protein provided herein comprises an IL-33 domain, an ST2 domain, first and second anti-PD-L1 V_{H}H sdAbs, an Fc domain comprising a first and second peptide chains, and optionally first and second peptide linkers; wherein the C-terminus of the first anti-PD-L1 V_{H}H sdAb is connected to the N-terminus of the first peptide chain of the Fc domain directly or via a peptide linker, the C-terminus of the first peptide chain of the Fc domain is connected to the N-terminus of the ST2 domain directly or via the first peptide linker, and the C-terminus of the ST2 domain is connected to the N-terminus of the IL-33 domain directly or via the second peptide linker; and wherein the C-terminus of the second anti-PD-L1 V_{H}H sdAb is connected to the N-terminus of the second peptide chain of the Fc domain directly or via a peptide linker.

In another embodiment, the fusion protein provided herein comprises an IL-33 domain, an ST2 domain, first and second anti-PD-L1 V_{H}H sdAbs, an Fc domain comprising a first and second peptide chains, and first and second peptide linkers; wherein the C-terminus of the first anti-PD-L1 V_{H}H sdAb is connected to the N-terminus of the first peptide chain of the Fc domain directly or via a peptide linker, the C-terminus of the first peptide chain of the Fc domain is connected to the N-terminus of the ST2 domain via the first peptide linker, and the C-terminus of the ST2 domain is connected to the N-terminus of the IL-33 domain via the second peptide linker; and wherein the C-terminus of the second anti-PD-L1 V_{H}H sdAb is connected to the N-terminus of the second peptide chain of the Fc domain directly or via a peptide linker.

In yet another embodiment, the fusion protein provided herein comprises an IL-33 domain, an ST2 domain, first and second anti-PD-L1 V_{H}H sdAbs, an Fc domain comprising a first and second peptide chains, and optionally first and second peptide linkers; wherein the C-terminus of the first anti-PD-L1 V_{H}H single domain is connected to the N-terminus of the first peptide chain of the Fc domain directly or via a peptide linker, and the C-terminus of the first peptide chain of the Fc domain is connected to the N-terminus of the IL-33 domain directly or via the first peptide linker; and wherein the C-terminus of the second anti-PD-L1 V_{H}H sdAb is connected to the N-terminus of the second peptide chain of the Fc domain directly or via a peptide linker, and the C-terminus of the second peptide chain of the Fc domain is connected to the N-terminus of the ST2 domain directly or via the second peptide linker.

In still another embodiment, the fusion protein provided herein comprises an IL-33 domain, an ST2 domain, first and second anti-PD-L1 V_{H}H sdAbs, an Fc domain comprising a first and second peptide chains, and first and second peptide linkers; wherein the C-terminus of the first anti-PD-L1 V_{H}H sdAb is connected to the N-terminus of the first peptide chain of the Fc domain directly or via a peptide linker, and the C-terminus of the first peptide chain of the Fc domain is connected to the N-terminus of the IL-33 domain via the first peptide linker; and wherein the C-terminus of the second anti-PD-L1 V_{H}H sdAb is connected to the N-terminus of the second peptide chain of the Fc domain directly or via a peptide linker, and the C-terminus of the second peptide chain of the Fc domain is connected to the N-terminus of the ST2 domain via the second peptide linker.

In another embodiment, provided herein is a fusion protein comprising two IL-33 domains, two ST2 domains, two anti-PD-L1 V_{H}H sdAbs, and an Fc domain.

In one embodiment, the fusion protein provided herein comprises first and second IL-33 domains, first and second ST2 domains, first and second anti-PD-L1 V_{H}H sdAbs, an Fc domain comprising a first and second peptide chains, and optionally first, second, third, and fourth peptide linkers; wherein the C-terminus of the first anti-PD-L1 V_{H}H sdAb is connected to the N-terminus of the first peptide chain of the Fc domain directly or via a peptide linker, the C-terminus of the first peptide chain of the Fc domain is connected to the N-terminus of the first ST2 domain directly or via the first peptide linker, and the C-terminus of the first ST2 domain is connected to the N-terminus of the first IL-33 domain directly or via the second peptide linker; and wherein the C-terminus of the second anti-PD-L1 V_{H}H sdAb is connected to the N-terminus of the second peptide chain of the Fc domain directly or via a peptide linker, the C-terminus of the second peptide chain of the Fc domain is connected to the N-terminus of the second ST2 domain directly or via the third peptide linker, and the C-terminus of the second ST2 domain is connected to the N-terminus of the second IL-33 domain directly or via the fourth peptide linker.

In another embodiment, the fusion protein provided herein comprises first and second IL-33 domains, first and second ST2 domains, first and second anti-PD-L1 V_{H}H sdAbs, an Fc domain comprising a first and second peptide chains, and optionally first, second, third, and fourth peptide linkers; wherein the C-terminus of the first anti-PD-L1 V_{H}H sdAb is connected to the N-terminus of the first peptide chain of the Fc domain directly or via a peptide linker, the C-terminus of the first peptide chain of the Fc domain is connected to the N-terminus of the first ST2 domain via the first peptide linker, and the C-terminus of the first ST2 domain is connected to the N-terminus of the first IL-33 domain via the second peptide linker; and wherein the C-terminus of the second anti-PD-L1 V_{H}H sdAb is connected to the N-terminus of the second peptide chain of the Fc domain directly or via a peptide linker, the C-terminus of the second peptide chain of the Fc domain is connected to the N-terminus of the second ST2 domain via the third peptide linker, and the C-terminus of the second ST2 domain is connected to the N-terminus of the second IL-33 domain via the fourth peptide linker.

In one embodiment, each anti-PD-L1 V_{H}H sdAb comprises a CDR1 of SEQ ID NO: 113, a CDR2 of SEQ ID NO: 114, and a CDR3 of SEQ ID NO: 115. In another embodiment, each anti-PD-L1 V_{H}H sdAb comprises the amino acid sequence of SEQ ID NO: 116.

In certain embodiments, the anti-PD-L1 V_{H}H sdAb is a human antibody. In certain embodiments, the anti-PD-L1 V_{H}H sdAb is a humanized antibody.

In one embodiment, provided herein is a fusion protein comprising an IL-33 domain, an ST2 domain, a mesothelin (MSLN) binding domain, and an Fc domain.

In another embodiment, provided herein is a fusion protein comprising an IL-33 domain, an ST2 domain, two MSLN binding domains, and an Fc domain.

In one embodiment, the fusion protein provided herein comprises an IL-33 domain, an ST2 domain, first and second MSLN binding domains, an Fc domain comprising a first and second peptide chains, and optionally first and second peptide linkers; wherein a C-terminus of the first MSLN binding domain is connected to the N-terminus of the first peptide chain of the Fc domain directly or via a peptide linker, a C-terminus of the second MSLN binding domain is connected to the N-terminus of the second peptide chain of the Fc domain directly or via a peptide linker, the N-terminus of the ST2 domain is connected to the C-terminus of the first peptide chain of the Fc domain directly or via the first peptide linker, and the N-terminus of the IL-33 domain is connected to the C-terminus of the ST2 domain directly or via the second peptide linker.

In another embodiment, the fusion protein provided herein comprises an IL-33 domain, an ST2 domain, first and second MSLN binding domains, an Fc domain comprising a first and second peptide chains, and first and second peptide linkers; wherein a C-terminus of the first MSLN binding domain is connected to the N-terminus of the first peptide chain of the Fc domain directly or via a peptide linker, a C-terminus of the second MSLN binding domain is connected to the N-terminus of the second peptide chain of the Fc domain directly or via a peptide linker, the N-terminus of the ST2 domain is connected to the C-terminus of the first peptide chain of the Fc domain via the first peptide linker, and the N-terminus of the IL-33 domain is connected to the C-terminus of the ST2 domain via the second peptide linker.

In yet another embodiment, the fusion protein provided herein comprises an IL-33 domain, an ST2 domain, first and second MSLN binding domains, an Fc domain comprising a first and second peptide chains, and optionally first and second peptide linkers; wherein a C-terminus of the first MSLN binding domain is connected to the N-terminus of the first peptide chain of the Fc domain directly or via a peptide linker, a C-terminus of the second MSLN binding domain is connected to the N-terminus of the second peptide chain of the Fc domain directly or via a peptide linker, the N-terminus of the IL-33 domain is connected to the C-terminus of the first peptide chain of the Fc domain directly or via the first peptide linker, and the N-terminus of the ST2 domain is connected to the C-terminus of the second peptide chain of the Fc domain directly or via the second peptide linker.

In still another embodiment, the fusion protein provided herein comprises an IL-33 domain, an ST2 domain, first and second MSLN binding domains, an Fc domain comprising a first and second peptide chains, and first and second peptide linkers; wherein a C-terminus of the first MSLN binding domain is connected to the N-terminus of the first peptide chain of the Fc domain directly or via a peptide linker, a C-terminus of the second MSLN binding domain is connected to the N-terminus of the second peptide chain of the Fc domain directly or via a peptide linker, the N-terminus of the IL-33 domain is connected to the C-terminus of the first peptide chain of the Fc domain via the first peptide linker, and the N-terminus of the ST2 domain is connected to the C-terminus of the second peptide chain of the Fc domain via the second peptide linker.

In yet another embodiment, provided herein is a fusion protein comprising two IL-33 domains, two ST2 domains, two MSLN binding domains, and an Fc domain.

In one embodiment, the fusion protein provided herein comprises first and second IL-33 domains, first and second ST2 domains, first and second MSLN binding domains, an Fc domain comprising a first and second peptide chains, and optionally first, second, third, and fourth peptide linkers; wherein a C-terminus of the first MSLN binding domain is connected to the N-terminus of the first peptide chain of the Fc domain directly or via a peptide linker, and a C-terminus of the second MSLN binding domain is connected to the N-terminus of the second peptide chain of the Fc domain directly or via a peptide linker; wherein the N-terminus of the first ST2 domain is connected to the C-terminus of the first peptide chain of the Fc domain directly or via the first peptide linker, and the N-terminus of the first IL-33 domain is connected to the C-terminus of the first ST2 domain directly or via the second peptide linker; and wherein the N-terminus of the second ST2 domain is connected to the C-terminus of the second peptide chain of the Fc domain directly or via the third peptide linker, and the N-terminus of the second IL-33 domain is connected to the C-terminus of the second ST2 domain directly or via the fourth peptide linker.

In another embodiment, the fusion protein provided herein comprises first and second IL-33 domains, first and second ST2 domains, first and second MSLN binding domains, an Fc domain comprising a first and second peptide chains, and first, second, third, and fourth peptide linkers; wherein a C-terminus of the first MSLN binding domain is connected to the N-terminus of the first peptide chain of the Fc domain directly or via a peptide linker, and a C-terminus of the second MSLN binding domain is connected to the N-terminus of the second peptide chain of the Fc domain directly or via a peptide linker; wherein the N-terminus of the first ST2 domain is connected to the C-terminus of the first peptide chain of the Fc domain via the first peptide linker, and the N-terminus of the first IL-33 domain is connected to the C-terminus of the first ST2 domain via the second peptide linker; and wherein the N-terminus of the second ST2 domain is connected to the C-terminus of the second peptide chain of the Fc domain via the third peptide linker, and the N-terminus of the second IL-33 domain is connected to the C-terminus of the second ST2 domain via the fourth peptide linker.

In one embodiment, the MSLN binding domain is a single-chain variable fragment (scFv), Fab, Fab′, F(ab)₂, F(ab′)₂, Fv, diabody, triabody, tetrabody, minibody, or a V_{H}H sdAb.

In one embodiment, the MSLN binding domain is an scFv. In another embodiment, the MSLN binding domain is an scFv comprising a light chain (V_{L}), a heavy chain (V_{H}), and optionally a peptide linker, wherein the C-terminal of the light chain is connected to the N-terminal of the heavy chain directly or via the peptide linker, or wherein the N-terminal of the light chain is connected to the C-terminal of the heavy chain directly or via the peptide linker. In yet another embodiment, the MSLN binding domain is an scFv comprising a light chain (V_{L}), a heavy chain (V_{H}), and a peptide linker, wherein the C-terminal of the light chain is connected to the N-terminal of the heavy chain via the peptide linker, or wherein the N-terminal of the light chain is connected to the C-terminal of the heavy chain via the peptide linker. In yet another embodiment, the MSLN binding domain is an scFv comprising a light chain (V_{L}), a heavy chain (V_{H}), and a peptide linker, wherein the C-terminal of the light chain is connected to the N-terminal of the heavy chain via the peptide linker. In still another embodiment, the MSLN binding domain is an scFv comprising a light chain (V_{L}), a heavy chain (V_{H}), and a peptide linker, wherein the N-terminal of the light chain is connected to the C-terminal of the heavy chain via the peptide linker.

In one embodiment, the MSLN binding domain is a Fab. In another embodiment, the MSLN binding domain is a Fab′. In yet another embodiment, the MSLN binding domain is a F(ab)₂. In yet another embodiment, the MSLN binding domain is a F(ab′)₂. In yet another embodiment, the MSLN binding domain is a Fv. In yet another embodiment, the MSLN binding domain is a diabody. In yet another embodiment, the MSLN binding domain is a triabody. In yet another embodiment, the MSLN binding domain is a tetrabody. In yet another embodiment, the MSLN binding domain is a minibody. In yet another embodiment, the MSLN binding domain is an sdAb. In still another embodiment, the MSLN binding domain is a V_{H}H sdAb.

In one embodiment, the MSLN binding domain and Fc domain are parts of an intact anti-MSLN antibody comprising two light chains and two heavy chains.

Thus, in one embodiment, provided herein is a fusion protein comprising an IL-33 domain, an ST2 domain, and an intact anti-MSLN antibody comprising two light chains and two heavy chains.

In one embodiment, the fusion protein provided herein comprises an IL-33 domain, an ST2 domain, an intact anti-MSLN antibody comprising first and second light chains and first and second heavy chains, and optionally first and second peptide linkers; wherein the N-terminus of the ST2 domain is connected to the C-terminus of the first heavy chain of the intact anti-MSLN antibody directly or via the first peptide linker, and the N-terminus of the IL-33 domain is connected to the C-terminus of the ST2 domain directly or via the second peptide linker.

In another embodiment, the fusion protein provided herein comprises an IL-33 domain, an ST2 domain, an intact anti-MSLN antibody comprising first and second light chains and first and second heavy chains, and first and second peptide linkers; wherein the N-terminus of the ST2 domain is connected to the C-terminus of the first heavy chain of the intact anti-MSLN antibody via the first peptide linker, and the N-terminus of the IL-33 domain is connected to the C-terminus of the ST2 domain via the second peptide linker.

In yet another embodiment, the fusion protein provided herein comprises an IL-33 domain, an ST2 domain, an intact anti-MSLN antibody comprising first and second light chains and first and second heavy chains, and optionally first and second peptide linkers; wherein the N-terminus of the ST2 domain is connected to the C-terminus of the first heavy chain of the intact anti-MSLN antibody directly or via the first peptide linker, and the N-terminus of the IL-33 domain is connected to the C-terminus of the second heavy chain of the intact anti-MSLN antibody directly or via the second peptide linker.

In still another embodiment, the fusion protein provided herein comprises an IL-33 domain, an ST2 domain, an intact anti-MSLN antibody comprising first and second light chains and first and second heavy chains, and first and second peptide linkers; wherein the N-terminus of the ST2 domain is connected to the C-terminus of the first heavy chain of the intact anti-MSLN antibody via the first peptide linker, and the N-terminus of the IL-33 domain is connected to the C-terminus of the second heavy chain of the intact anti-MSLN antibody via the second peptide linker.

In another embodiment, provided herein is a fusion protein comprising two IL-33 domains, two ST2 domains, and an intact anti-MSLN antibody comprising two light chains and two heavy chains.

In one embodiment, the fusion protein provided herein comprises first and second IL-33 domains, first and second ST2 domains, an intact anti-MSLN antibody comprising first and second light chains and first and second heavy chains, and optionally first, second, third, and fourth peptide linkers; wherein the N-terminus of the first ST2 domain is connected to the C-terminus of the first heavy chain of the intact anti-MSLN antibody directly or via the first peptide linker, and the N-terminus of the first IL-33 domain is connected to the C-terminus of the first ST2 domain directly or via the second peptide linker; and wherein the N-terminus of the second ST2 domain is connected to the C-terminus of the second heavy chain of the intact anti-MSLN antibody directly or via the third peptide linker, and the N-terminus of the second IL-33 domain is connected to the C-terminus of the second ST2 domain directly or via the fourth peptide linker.

In another embodiment, the fusion protein provided herein comprises first and second IL-33 domains, first and second ST2 domains, an intact anti-MSLN antibody comprising first and second light chains and first and second heavy chains, and optionally first, second, third, and fourth peptide linkers; wherein the N-terminus of the first ST2 domain is connected to the C-terminus of the first heavy chain of the intact anti-MSLN antibody via the first peptide linker, and the N-terminus of the first IL-33 domain is connected to the C-terminus of the first ST2 domain via the second peptide linker; and wherein the N-terminus of the second ST2 domain is connected to the C-terminus of the second heavy chain of the intact anti-MSLN antibody via the third peptide linker, and the N-terminus of the second IL-33 domain is connected to the C-terminus of the second ST2 domain via the fourth peptide linker.

In one embodiment, the intact MSLN antibody is an IgA, IgD, IgE, IgG, or IgM antibody. In another embodiment, the intact MSLN antibody is an IgA antibody. In yet another embodiment, the intact MSLN antibody is an IgD antibody. In yet another embodiment, the intact MSLN antibody is an IgE antibody. In yet another embodiment, the intact MSLN antibody is an IgG antibody. In still another embodiment, the intact MSLN antibody is an IgM antibody.

In one embodiment, the intact MSLN antibody is an IgA1, IgA2, IgG1, IgG2, IgG3, or IgG4 antibody. In another embodiment, the intact MSLN antibody is an IgA1 or IgA2. In yet another embodiment, the intact MSLN antibody is an IgA1. In yet another embodiment, the intact MSLN antibody is an IgA2. In yet another embodiment, the intact MSLN antibody is an IgG1, IgG2, IgG3, or IgG4 antibody. In yet another embodiment, the intact MSLN antibody is an IgG1 antibody. In yet another embodiment, the intact MSLN antibody is an IgG2 antibody. In yet another embodiment, the intact MSLN antibody is an IgG3 antibody. In still another embodiment, the intact MSLN antibody is an IgG4 antibody.

In one embodiment, the light chain of the intact MSLN antibody is a kappa or lambda chain. In another embodiment, the light chain of the intact MSLN antibody is a kappa chain. In yet another embodiment, the light chain of the intact MSLN antibody is a lambda chain.

In one embodiment, the intact MSLN antibody is a human antibody. In another embodiment, the intact MSLN antibody is a humanized antibody.

In one embodiment, the intact MSLN antibody is an antibody with a “knobs-into-holes” mutation. In another embodiment, the intact MSLN antibody is a human IgG1 antibody with an N297A mutation. In yet another embodiment, the intact MSLN antibody is a human IgG1 antibody with a “knobs-into-holes” mutation. In still another embodiment, the intact MSLN antibody is a human IgG4 antibody with a “knobs-into-holes” mutation.

In one embodiment, the MSLN binding domain is an sdAb. In another embodiment, the MSLN binding domain is a V_{H}H sdAb.

Thus, in one embodiment, provided herein is a fusion protein comprising an IL-33 domain, an ST2 domain, an anti-MSLN V_{H}H sdAb, and an Fc domain.

In one embodiment, the fusion protein provided herein comprises an IL-33 domain, an ST2 domain, first and second anti-MSLN V_{H}H sdAbs, an Fc domain comprising a first and second peptide chains, and optionally first and second peptide linkers; wherein the C-terminus of the first anti-MSLN V_{H}H sdAb is connected to the N-terminus of the first peptide chain of the Fc domain directly or via a peptide linker, the C-terminus of the first peptide chain of the Fc domain is connected to the N-terminus of the ST2 domain directly or via the first peptide linker, and the C-terminus of the ST2 domain is connected to the N-terminus of the IL-33 domain directly or via the second peptide linker; and wherein the C-terminus of the second anti-MSLN V_{H}H sdAb is connected to the N-terminus of the second peptide chain of the Fc domain directly or via a peptide linker.

In another embodiment, the fusion protein provided herein comprises an IL-33 domain, an ST2 domain, first and second V_{H}H sdAbs, an Fc domain comprising a first and second peptide chains, and first and second peptide linkers; wherein the C-terminus of the first anti-MSLN V_{H}H sdAb is connected to the N-terminus of the first peptide chain of the Fc domain directly or via a peptide linker, the C-terminus of the first peptide chain of the Fc domain is connected to the N-terminus of the ST2 domain via the first peptide linker, and the C-terminus of the ST2 domain is connected to the N-terminus of the IL-33 domain via the second peptide linker; and wherein the C-terminus of the second anti-MSLN V_{H}H sdAb is connected to the N-terminus of the second peptide chain of the Fc domain directly or via a peptide linker.

In yet another embodiment, the fusion protein provided herein comprises an IL-33 domain, an ST2 domain, first and second anti-MSLN V_{H}H sdAbs, an Fc domain comprising a first and second peptide chains, and optionally first and second peptide linkers; wherein the C-terminus of the first anti-MSLN V_{H}H sdAb is connected to the N-terminus of the first peptide chain of the Fc domain directly or via a peptide linker, and the C-terminus of the first peptide chain of the Fc domain is connected to the N-terminus of the IL-33 domain directly or via the first peptide linker; and wherein the C-terminus of the second anti-MSLN V_{H}H sdAb is connected to the N-terminus of the second peptide chain of the Fc domain directly or via a peptide linker, and the C-terminus of the second peptide chain of the Fc domain is connected to the N-terminus of the ST2 domain directly or via the second peptide linker.

In still another embodiment, the fusion protein provided herein comprises an IL-33 domain, an ST2 domain, first and second anti-MSLN V_{H}H sdAbs, an Fc domain comprising a first and second peptide chains, and first and second peptide linkers; wherein the C-terminus of the first anti-MSLN V_{H}H sdAb is connected to the N-terminus of the first peptide chain of the Fc domain directly or via a peptide linker, and the C-terminus of the first peptide chain of the Fc domain is connected to the N-terminus of the IL-33 domain via the first peptide linker; and wherein the C-terminus of the second anti-MSLN V_{H}H sdAb is connected to the N-terminus of the second peptide chain of the Fc domain directly or via a peptide linker, and the C-terminus of the second peptide chain of the Fc domain is connected to the N-terminus of the ST2 domain via the second peptide linker.

In another embodiment, provided herein is a fusion protein comprising two IL-33 domains, two ST2 domains, two anti-MSLN V_{H}H sdAbs, and an Fc domain.

In one embodiment, the fusion protein provided herein comprises first and second IL-33 domains, first and second ST2 domains, first and second anti-MSLN V_{H}H sdAbs, an Fc domain comprising a first and second peptide chains, and optionally first, second, third, and fourth peptide linkers; wherein the C-terminus of the first anti-MSLN V_{H}H sdAb is connected to the N-terminus of the first peptide chain of the Fc domain directly or via a peptide linker, the C-terminus of the first peptide chain of the Fc domain is connected to the N-terminus of the first ST2 domain directly or via the first peptide linker, and the C-terminus of the first ST2 domain is connected to the N-terminus of the first IL-33 domain directly or via the second peptide linker; and wherein the C-terminus of the second anti-MSLN V_{H}H sdAb is connected to the N-terminus of the second peptide chain of the Fc domain directly or via a peptide linker, the C-terminus of the second peptide chain of the Fc domain is connected to the N-terminus of the second ST2 domain directly or via the third peptide linker, and the C-terminus of the second ST2 domain is connected to the N-terminus of the second IL-33 domain directly or via the fourth peptide linker.

In another embodiment, the fusion protein provided herein comprises first and second IL-33 domains, first and second ST2 domains, first and second anti-MSLN V_{H}H sdAbs, an Fc domain comprising a first and second peptide chains, and optionally first, second, third, and fourth peptide linkers; wherein the C-terminus of the first anti-MSLN V_{H}H sdAb is connected to the N-terminus of the first peptide chain of the Fc domain directly or via a peptide linker, the C-terminus of the first peptide chain of the Fc domain is connected to the N-terminus of the first ST2 domain via the first peptide linker, and the C-terminus of the first ST2 domain is connected to the N-terminus of the first IL-33 domain via the second peptide linker; and wherein the C-terminus of the second anti-MSLN V_{H}H sdAb is connected to the N-terminus of the second peptide chain of the Fc domain directly or via a peptide linker, the C-terminus of the second peptide chain of the Fc domain is connected to the N-terminus of the second ST2 domain via the third peptide linker, and the C-terminus of the second ST2 domain is connected to the N-terminus of the second IL-33 domain via the fourth peptide linker.

In one embodiment, each anti-MSLN V_{H}H sdAb comprises a peptide chain comprising a CDR1 of SEQ ID NO: 117, a CDR2 of SEQ ID NO: 118, and a CDR3 of SEQ ID NO: 119. In another embodiment, each anti-MSLN V_{H}H sdAb independently comprises the amino acid of SEQ ID NO: 124.

In certain embodiments, the anti-MSLN antibody is a human antibody. In certain embodiments, the anti-MSLN antibody is a humanized antibody.

In one embodiment, each peptide linker is independently a peptide linker having an amino acid sequence of GSG or one of SEQ ID NOs: 16 to 54. In another embodiment, each peptide linker is independently a GSG linker having an amino acid sequence of GSG or SEQ ID NO: 16, 17, or 18. In yet another embodiment, each peptide linker is independently a G3S linker having an amino acid sequence of SEQ ID NO: 19, 20, 21, or 22. In yet another embodiment, each peptide linker is independently a G4S linker having an amino acid sequence of SEQ ID NO: 23, 24, 25, or 26. In yet another embodiment, each peptide linker is independently an SGSG linker having an amino acid sequence of SEQ ID NO: 27, 28, 29, or 30. In yet another embodiment, each peptide linker is independently an SG3S linker having an amino acid sequence of SEQ ID NO: 31, 32, 33, or 34. In yet another embodiment, each peptide linker is independently an SG4S linker having an amino acid sequence of SEQ ID NO: 35, 36, 37, or 38. In yet another embodiment, each peptide linker is independently an EAAAK linker having an amino acid sequence of SEQ ID NO: 39, 40, 41, or 42. In yet another embodiment, each peptide linker is independently a PAPAP linker having an amino acid sequence of SEQ ID NO: 43, 44, 45, or 46. In yet another embodiment, each peptide linker is independently a VLVH linker having an amino acid sequence of SEQ ID NO: 47. In yet another embodiment, each peptide linker is independently a RAKPS linker having an amino acid sequence of SEQ ID NO: 48 or 49. In yet another embodiment, each peptide linker is independently an ASTKG linker having an amino acid sequence of SEQ ID NO: 50 or 51. In yet another embodiment, each peptide linker is independently an AKTHT linker having an amino acid sequence of SEQ ID NO: 52 or 53. In still another embodiment, each peptide linker is independently a linker having an amino acid sequence of SEQ ID NO: 54.

In one embodiment, each peptide linker is independently a non-cleavable linker. In another embodiment, each peptide linker is independently a non-cleavable linker having an amino acid sequence of GSG or one of SEQ ID NOs: 16 to 53. In yet another embodiment, each peptide linker is independently a cleavable linker. In still another embodiment, each peptide linker is a cleavable linker having an amino acid sequence of SEQ ID NO: 54.

In one embodiment, the first peptide linker is independently a peptide linker having an amino acid sequence of GSG or one of SEQ ID NOs: 16 to 54. In another embodiment, the first peptide linker is independently a GSG linker having an amino acid sequence of GSG or SEQ ID NO: 16, 17, or 18. In yet another embodiment, the first peptide linker is independently a G3S linker having an amino acid sequence of SEQ ID NO: 19, 20, 21, or 22. In yet another embodiment, the first peptide linker is independently a G4S linker having an amino acid sequence of SEQ ID NO: 23, 24, 25, or 26. In yet another embodiment, the first peptide linker is independently an SGSG linker having an amino acid sequence of SEQ ID NO: 27, 28, 29, or 30. In yet another embodiment, the first peptide linker is independently an SG3S linker having an amino acid sequence of SEQ ID NO: 31, 32, 33, or 34. In yet another embodiment, the first peptide linker is independently an SG4S linker having an amino acid sequence of SEQ ID NO: 35, 36, 37, or 38. In yet another embodiment, the first peptide linker is independently an EAAAK linker having an amino acid sequence of SEQ ID NO: 39, 40, 41, or 42. In yet another embodiment, the first peptide linker is independently a PAPAP linker having an amino acid sequence of SEQ ID NO: 43, 44, 45, or 46. In yet another embodiment, the first peptide linker is independently a VLVH linker having an amino acid sequence of SEQ ID NO: 47. In yet another embodiment, the first peptide linker is independently a RAKPS linker having an amino acid sequence of SEQ ID NO: 48 or 49. In yet another embodiment, the first peptide linker is independently an ASTKG linker having an amino acid sequence of SEQ ID NO: 50 or 51. In yet another embodiment, the first peptide linker is independently an AKTHT linker having an amino acid sequence of SEQ ID NO: 52 or 53. In still another embodiment, the first peptide linker is independently a linker having an amino acid sequence of SEQ ID NO: 54.

In one embodiment, the first peptide linker is a non-cleavable linker. In another embodiment, the first peptide linker is a non-cleavable linker having an amino acid sequence of GSG or one of SEQ ID NOs: 16 to 53. In yet another embodiment, the first peptide linker is a cleavable linker. In still another embodiment, the first peptide linker is a cleavable linker having an amino acid sequence of SEQ ID NO: 54.

In one embodiment, the second peptide linker is independently a peptide linker having an amino acid sequence of GSG or one of SEQ ID NOs: 16 to 54. In another embodiment, the second peptide linker is independently a GSG linker having an amino acid sequence of GSG or SEQ ID NO: 16, 17, or 18. In yet another embodiment, the second peptide linker is independently a G3S linker having an amino acid sequence of SEQ ID NO: 19, 20, 21, or 22. In yet another embodiment, the second peptide linker is independently a G4S linker having an amino acid sequence of SEQ ID NO: 23, 24, 25, or 26. In yet another embodiment, the second peptide linker is independently an SGSG linker having an amino acid sequence of SEQ ID NO: 27, 28, 29, or 30. In yet another embodiment, the second peptide linker is independently an SG3S linker having an amino acid sequence of SEQ ID NO: 31, 32, 33, or 34. In yet another embodiment, the second peptide linker is independently an SG4S linker having an amino acid sequence of SEQ ID NO: 35, 36, 37, or 38. In yet another embodiment, the second peptide linker is independently an EAAAK linker having an amino acid sequence of SEQ ID NO: 39, 40, 41, or 42. In yet another embodiment, the second peptide linker is independently a PAPAP linker having an amino acid sequence of SEQ ID NO: 43, 44, 45, or 46. In yet another embodiment, the second peptide linker is independently a VLVH linker having an amino acid sequence of SEQ ID NO: 47. In yet another embodiment, the second peptide linker is independently a RAKPS linker having an amino acid sequence of SEQ ID NO: 48 or 49. In yet another embodiment, the second peptide linker is independently an ASTKG linker having an amino acid sequence of SEQ ID NO: 50 or 51. In yet another embodiment, the second peptide linker is independently an AKTHT linker having an amino acid sequence of SEQ ID NO: 52 or 53. In still another embodiment, the second peptide linker is independently a linker having an amino acid sequence of SEQ ID NO: 54.

In one embodiment, the second peptide linker is a non-cleavable linker. In another embodiment, the second peptide linker is a non-cleavable linker having an amino acid sequence of GSG or one of SEQ ID NOs: 16 to 53. In yet another embodiment, the second peptide linker is a cleavable linker. In still another embodiment, the second peptide linker is a cleavable linker having an amino acid sequence of SEQ ID NO: 54.

In one embodiment, the third peptide linker is independently a peptide linker having an amino acid sequence of GSG or one of SEQ ID NOs: 16 to 54. In another embodiment, the third peptide linker is independently a GSG linker having an amino acid sequence of GSG or SEQ ID NO: 16, 17, or 18. In yet another embodiment, the third peptide linker is independently a G3S linker having an amino acid sequence of SEQ ID NO: 19, 20, 21, or 22. In yet another embodiment, the third peptide linker is independently a G4S linker having an amino acid sequence of SEQ ID NO: 23, 24, 25, or 26. In yet another embodiment, the third peptide linker is independently an SGSG linker having an amino acid sequence of SEQ ID NO: 27, 28, 29, or 30. In yet another embodiment, the third peptide linker is independently an SG3S linker having an amino acid sequence of SEQ ID NO: 31, 32, 33, or 34. In yet another embodiment, the third peptide linker is independently an SG4S linker having an amino acid sequence of SEQ ID NO: 35, 36, 37, or 38. In yet another embodiment, the third peptide linker is independently an EAAAK linker having an amino acid sequence of SEQ ID NO: 39, 40, 41, or 42. In yet another embodiment, the third peptide linker is independently a PAPAP linker having an amino acid sequence of SEQ ID NO: 43, 44, 45, or 46. In yet another embodiment, the third peptide linker is independently a VLVH linker having an amino acid sequence of SEQ ID NO: 47. In yet another embodiment, the third peptide linker is independently a RAKPS linker having an amino acid sequence of SEQ ID NO: 48 or 49. In yet another embodiment, the third peptide linker is independently an ASTKG linker having an amino acid sequence of SEQ ID NO: 50 or 51. In yet another embodiment, the third peptide linker is independently an AKTHT linker having an amino acid sequence of SEQ ID NO: 52 or 53. In still another embodiment, the third peptide linker is independently a linker having an amino acid sequence of SEQ ID NO: 54.

In one embodiment, the third peptide linker is a non-cleavable linker. In another embodiment, the third peptide linker is a non-cleavable linker having an amino acid sequence of GSG or one of SEQ ID NOs: 16 to 53. In yet another embodiment, the third peptide linker is a cleavable linker. In still another embodiment, the third peptide linker is a cleavable linker having an amino acid sequence of SEQ ID NO: 54.

In one embodiment, the fourth peptide linker is independently a peptide linker having an amino acid sequence of GSG or one of SEQ ID NOs: 16 to 54. In another embodiment, the fourth peptide linker is independently a GSG linker having an amino acid sequence of GSG or SEQ ID NO: 16, 17, or 18. In yet another embodiment, the fourth peptide linker is independently a G3S linker having an amino acid sequence of SEQ ID NO: 19, 20, 21, or 22. In yet another embodiment, the fourth peptide linker is independently a G4S linker having an amino acid sequence of SEQ ID NO: 23, 24, 25, or 26. In yet another embodiment, the fourth peptide linker is independently an SGSG linker having an amino acid sequence of SEQ ID NO: 27, 28, 29, or 30. In yet another embodiment, the fourth peptide linker is independently an SG3S linker having an amino acid sequence of SEQ ID NO: 31, 32, 33, or 34. In yet another embodiment, the fourth peptide linker is independently an SG4S linker having an amino acid sequence of SEQ ID NO: 35, 36, 37, or 38. In yet another embodiment, the fourth peptide linker is independently an EAAAK linker having an amino acid sequence of SEQ ID NO: 39, 40, 41, or 42. In yet another embodiment, the fourth peptide linker is independently a PAPAP linker having an amino acid sequence of SEQ ID NO: 43, 44, 45, or 46. In yet another embodiment, the fourth peptide linker is independently a VLVH linker having an amino acid sequence of SEQ ID NO: 47. In yet another embodiment, the fourth peptide linker is independently a RAKPS linker having an amino acid sequence of SEQ ID NO: 48 or 49. In yet another embodiment, the fourth peptide linker is independently an ASTKG linker having an amino acid sequence of SEQ ID NO: 50 or 51. In yet another embodiment, the fourth peptide linker is independently an AKTHT linker having an amino acid sequence of SEQ ID NO: 52 or 53. In still another embodiment, the fourth peptide linker is independently a linker having an amino acid sequence of SEQ ID NO: 54.

In one embodiment, the fourth peptide linker is a non-cleavable linker. In another embodiment, the fourth peptide linker is a non-cleavable linker having an amino acid sequence of GSG or one of SEQ ID NOs: 16 to 53. In yet another embodiment, the fourth peptide linker is a cleavable linker. In still another embodiment, the fourth peptide linker is a cleavable linker having an amino acid sequence of SEQ ID NO: 54.

In one embodiment, the IL-33 domain comprises an amino acid sequence of SEQ ID NO: 125, 126, 127, 128, or 129; or a mutein or variant thereof. In another embodiment, the IL-33 domain comprises an amino acid sequence of SEQ ID NO: 125, or a mutein or variant thereof. In yet another embodiment, the IL-33 domain comprises an amino acid sequence of SEQ ID NO: 126, or a mutein or variant thereof. In yet another embodiment, the IL-33 domain comprises an amino acid sequence of SEQ ID NO: 127, or a mutein or variant thereof. In yet another embodiment, the IL-33 domain comprises an amino acid sequence of SEQ ID NO: 128, or a mutein or variant thereof. In still another embodiment, the IL-33 domain comprises an amino acid sequence of SEQ ID NO: 129, or a mutein or variant thereof.

In certain embodiments, the IL-33 domain comprises an amino acid sequence that is no less than about 70%, no less than about 75%, no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 125.

In certain embodiments, the IL-33 domain comprises an amino acid sequence that is no less than about 70% identical to the amino acid sequence of SEQ ID NO: 125. In certain embodiments, the IL-33 domain comprises an amino acid sequence that is no less than about 75% identical to the amino acid sequence of SEQ ID NO: 125. In certain embodiments, the IL-33 domain comprises an amino acid sequence that is no less than about 80% identical to the amino acid sequence of SEQ ID NO: 125. In certain embodiments, the IL-33 domain comprises an amino acid sequence that is no less than about 85% identical to the amino acid sequence of SEQ ID NO: 125. In certain embodiments, the IL-33 domain comprises an amino acid sequence that is no less than about 90% identical to the amino acid sequence of SEQ ID NO: 125. In certain embodiments, the IL-33 domain comprises an amino acid sequence that is no less than about 91% identical to the amino acid sequence of SEQ ID NO: 125. In certain embodiments, the IL-33 domain comprises an amino acid sequence that is no less than about 92% identical to the amino acid sequence of SEQ ID NO: 125. In certain embodiments, the IL-33 domain comprises an amino acid sequence that is no less than about 93% identical to the amino acid sequence of SEQ ID NO: 125. In certain embodiments, the IL-33 domain comprises an amino acid sequence that is no less than about 94% identical to the amino acid sequence of SEQ ID NO: 125. In certain embodiments, the IL-33 domain comprises an amino acid sequence that is no less than about 95% identical to the amino acid sequence of SEQ ID NO: 125. In certain embodiments, the IL-33 domain comprises an amino acid sequence that is no less than about 96% identical to the amino acid sequence of SEQ ID NO: 125. In certain embodiments, the IL-33 domain comprises an amino acid sequence that is no less than about 97% identical to the amino acid sequence of SEQ ID NO: 125. In certain embodiments, the IL-33 domain comprises an amino acid sequence that is no less than about 98% identical to the amino acid sequence of SEQ ID NO: 125. In certain embodiments, the IL-33 domain comprises an amino acid sequence that is no less than about 99% identical to the amino acid sequence of SEQ ID NO: 125.

In certain embodiments, the IL-33 domain comprises an amino acid sequence that is no less than about 70%, no less than about 75%, no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 126.

In certain embodiments, the IL-33 domain comprises an amino acid sequence that is no less than about 70% identical to the amino acid sequence of SEQ ID NO: 126. In certain embodiments, the IL-33 domain comprises an amino acid sequence that is no less than about 75% identical to the amino acid sequence of SEQ ID NO: 126. In certain embodiments, the IL-33 domain comprises an amino acid sequence that is no less than about 80% identical to the amino acid sequence of SEQ ID NO: 126. In certain embodiments, the IL-33 domain comprises an amino acid sequence that is no less than about 85% identical to the amino acid sequence of SEQ ID NO: 126. In certain embodiments, the IL-33 domain comprises an amino acid sequence that is no less than about 90% identical to the amino acid sequence of SEQ ID NO: 126. In certain embodiments, the IL-33 domain comprises an amino acid sequence that is no less than about 91% identical to the amino acid sequence of SEQ ID NO: 126. In certain embodiments, the IL-33 domain comprises an amino acid sequence that is no less than about 92% identical to the amino acid sequence of SEQ ID NO: 126. In certain embodiments, the IL-33 domain comprises an amino acid sequence that is no less than about 93% identical to the amino acid sequence of SEQ ID NO: 126. In certain embodiments, the IL-33 domain comprises an amino acid sequence that is no less than about 94% identical to the amino acid sequence of SEQ ID NO: 126. In certain embodiments, the IL-33 domain comprises an amino acid sequence that is no less than about 95% identical to the amino acid sequence of SEQ ID NO: 126. In certain embodiments, the IL-33 domain comprises an amino acid sequence that is no less than about 96% identical to the amino acid sequence of SEQ ID NO: 126. In certain embodiments, the IL-33 domain comprises an amino acid sequence that is no less than about 97% identical to the amino acid sequence of SEQ ID NO: 126. In certain embodiments, the IL-33 domain comprises an amino acid sequence that is no less than about 98% identical to the amino acid sequence of SEQ ID NO: 126. In certain embodiments, the IL-33 domain comprises an amino acid sequence that is no less than about 99% identical to the amino acid sequence of SEQ ID NO: 126.

In certain embodiments, the IL-33 domain comprises an amino acid sequence that is no less than about 70%, no less than about 75%, no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 127.

In certain embodiments, the IL-33 domain comprises an amino acid sequence that is no less than about 70% identical to the amino acid sequence of SEQ ID NO: 127. In certain embodiments, the IL-33 domain comprises an amino acid sequence that is no less than about 75% identical to the amino acid sequence of SEQ ID NO: 127. In certain embodiments, the IL-33 domain comprises an amino acid sequence that is no less than about 80% identical to the amino acid sequence of SEQ ID NO: 127. In certain embodiments, the IL-33 domain comprises an amino acid sequence that is no less than about 85% identical to the amino acid sequence of SEQ ID NO: 127. In certain embodiments, the IL-33 domain comprises an amino acid sequence that is no less than about 90% identical to the amino acid sequence of SEQ ID NO: 127. In certain embodiments, the IL-33 domain comprises an amino acid sequence that is no less than about 91% identical to the amino acid sequence of SEQ ID NO: 127. In certain embodiments, the IL-33 domain comprises an amino acid sequence that is no less than about 92% identical to the amino acid sequence of SEQ ID NO: 127. In certain embodiments, the IL-33 domain comprises an amino acid sequence that is no less than about 93% identical to the amino acid sequence of SEQ ID NO: 127. In certain embodiments, the IL-33 domain comprises an amino acid sequence that is no less than about 94% identical to the amino acid sequence of SEQ ID NO: 127. In certain embodiments, the IL-33 domain comprises an amino acid sequence that is no less than about 95% identical to the amino acid sequence of SEQ ID NO: 127. In certain embodiments, the IL-33 domain comprises an amino acid sequence that is no less than about 96% identical to the amino acid sequence of SEQ ID NO: 127. In certain embodiments, the IL-33 domain comprises an amino acid sequence that is no less than about 97% identical to the amino acid sequence of SEQ ID NO: 127. In certain embodiments, the IL-33 domain comprises an amino acid sequence that is no less than about 98% identical to the amino acid sequence of SEQ ID NO: 127. In certain embodiments, the IL-33 domain comprises an amino acid sequence that is no less than about 99% identical to the amino acid sequence of SEQ ID NO: 127.

In certain embodiments, the IL-33 domain comprises an amino acid sequence that is no less than about 70%, no less than about 75%, no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 128.

In certain embodiments, the IL-33 domain comprises an amino acid sequence that is no less than about 70% identical to the amino acid sequence of SEQ ID NO: 128. In certain embodiments, the IL-33 domain comprises an amino acid sequence that is no less than about 75% identical to the amino acid sequence of SEQ ID NO: 128. In certain embodiments, the IL-33 domain comprises an amino acid sequence that is no less than about 80% identical to the amino acid sequence of SEQ ID NO: 128. In certain embodiments, the IL-33 domain comprises an amino acid sequence that is no less than about 85% identical to the amino acid sequence of SEQ ID NO: 128. In certain embodiments, the IL-33 domain comprises an amino acid sequence that is no less than about 90% identical to the amino acid sequence of SEQ ID NO: 128. In certain embodiments, the IL-33 domain comprises an amino acid sequence that is no less than about 91% identical to the amino acid sequence of SEQ ID NO: 128. In certain embodiments, the IL-33 domain comprises an amino acid sequence that is no less than about 92% identical to the amino acid sequence of SEQ ID NO: 128. In certain embodiments, the IL-33 domain comprises an amino acid sequence that is no less than about 93% identical to the amino acid sequence of SEQ ID NO: 128. In certain embodiments, the IL-33 domain comprises an amino acid sequence that is no less than about 94% identical to the amino acid sequence of SEQ ID NO: 128. In certain embodiments, the IL-33 domain comprises an amino acid sequence that is no less than about 95% identical to the amino acid sequence of SEQ ID NO: 128. In certain embodiments, the IL-33 domain comprises an amino acid sequence that is no less than about 96% identical to the amino acid sequence of SEQ ID NO: 128. In certain embodiments, the IL-33 domain comprises an amino acid sequence that is no less than about 97% identical to the amino acid sequence of SEQ ID NO: 128. In certain embodiments, the IL-33 domain comprises an amino acid sequence that is no less than about 98% identical to the amino acid sequence of SEQ ID NO: 128. In certain embodiments, the IL-33 domain comprises an amino acid sequence that is no less than about 99% identical to the amino acid sequence of SEQ ID NO: 128.

In certain embodiments, the IL-33 domain comprises an amino acid sequence that is no less than about 70%, no less than about 75%, no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 129.

In certain embodiments, the IL-33 domain comprises an amino acid sequence that is no less than about 70% identical to the amino acid sequence of SEQ ID NO: 129. In certain embodiments, the IL-33 domain comprises an amino acid sequence that is no less than about 75% identical to the amino acid sequence of SEQ ID NO: 129. In certain embodiments, the IL-33 domain comprises an amino acid sequence that is no less than about 80% identical to the amino acid sequence of SEQ ID NO: 129. In certain embodiments, the IL-33 domain comprises an amino acid sequence that is no less than about 85% identical to the amino acid sequence of SEQ ID NO: 129. In certain embodiments, the IL-33 domain comprises an amino acid sequence that is no less than about 90% identical to the amino acid sequence of SEQ ID NO: 129. In certain embodiments, the IL-33 domain comprises an amino acid sequence that is no less than about 91% identical to the amino acid sequence of SEQ ID NO: 129. In certain embodiments, the IL-33 domain comprises an amino acid sequence that is no less than about 92% identical to the amino acid sequence of SEQ ID NO: 129. In certain embodiments, the IL-33 domain comprises an amino acid sequence that is no less than about 93% identical to the amino acid sequence of SEQ ID NO: 129. In certain embodiments, the IL-33 domain comprises an amino acid sequence that is no less than about 94% identical to the amino acid sequence of SEQ ID NO: 129. In certain embodiments, the IL-33 domain comprises an amino acid sequence that is no less than about 95% identical to the amino acid sequence of SEQ ID NO: 129. In certain embodiments, the IL-33 domain comprises an amino acid sequence that is no less than about 96% identical to the amino acid sequence of SEQ ID NO: 129. In certain embodiments, the IL-33 domain comprises an amino acid sequence that is no less than about 97% identical to the amino acid sequence of SEQ ID NO: 129. In certain embodiments, the IL-33 domain comprises an amino acid sequence that is no less than about 98% identical to the amino acid sequence of SEQ ID NO: 129. In certain embodiments, the IL-33 domain comprises an amino acid sequence that is no less than about 99% identical to the amino acid sequence of SEQ ID NO: 129.

In certain embodiments, the IL-33 domain further includes one or more additional substitutions, deletions, and/or insertions.

In one embodiment, the ST2 domain comprises an amino acid sequence of SEQ ID NO: 130 or 131, or a mutein or variant thereof. In another embodiment, the ST2 domain comprises an amino acid sequence of SEQ ID NO: 130, or a mutein or variant thereof. In yet another embodiment, the ST2 domain comprises an amino acid sequence of SEQ ID NO: 131, or a mutein or variant thereof.

In certain embodiments, the ST2 domain comprises an amino acid sequence that is no less than about 70%, no less than about 75%, no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 130.

In certain embodiments, the ST2 domain comprises an amino acid sequence that is no less than about 70% identical to the amino acid sequence of SEQ ID NO: 130. In certain embodiments, the ST2 domain comprises an amino acid sequence that is no less than about 75% identical to the amino acid sequence of SEQ ID NO: 130. In certain embodiments, the ST2 domain comprises an amino acid sequence that is no less than about 80% identical to the amino acid sequence of SEQ ID NO: 130. In certain embodiments, the ST2 domain comprises an amino acid sequence that is no less than about 85% identical to the amino acid sequence of SEQ ID NO: 130. In certain embodiments, the ST2 domain comprises an amino acid sequence that is no less than about 90% identical to the amino acid sequence of SEQ ID NO: 130. In certain embodiments, the ST2 domain comprises an amino acid sequence that is no less than about 91% identical to the amino acid sequence of SEQ ID NO: 130. In certain embodiments, the ST2 domain comprises an amino acid sequence that is no less than about 92% identical to the amino acid sequence of SEQ ID NO: 130. In certain embodiments, the ST2 domain comprises an amino acid sequence that is no less than about 93% identical to the amino acid sequence of SEQ ID NO: 130. In certain embodiments, the ST2 domain comprises an amino acid sequence that is no less than about 94% identical to the amino acid sequence of SEQ ID NO: 130. In certain embodiments, the ST2 domain comprises an amino acid sequence that is no less than about 95% identical to the amino acid sequence of SEQ ID NO: 130. In certain embodiments, the ST2 domain comprises an amino acid sequence that is no less than about 96% identical to the amino acid sequence of SEQ ID NO: 130. In certain embodiments, the ST2 domain comprises an amino acid sequence that is no less than about 97% identical to the amino acid sequence of SEQ ID NO: 130. In certain embodiments, the ST2 domain comprises an amino acid sequence that is no less than about 98% identical to the amino acid sequence of SEQ ID NO: 130. In certain embodiments, the ST2 domain comprises an amino acid sequence that is no less than about 99% identical to the amino acid sequence of SEQ ID NO: 130.

In certain embodiments, the ST2 domain comprises an amino acid sequence that is no less than about 70%, no less than about 75%, no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 131.

In certain embodiments, the ST2 domain comprises an amino acid sequence that is no less than about 70% identical to the amino acid sequence of SEQ ID NO: 131. In certain embodiments, the ST2 domain comprises an amino acid sequence that is no less than about 75% identical to the amino acid sequence of SEQ ID NO: 131. In certain embodiments, the ST2 domain comprises an amino acid sequence that is no less than about 80% identical to the amino acid sequence of SEQ ID NO: 131. In certain embodiments, the ST2 domain comprises an amino acid sequence that is no less than about 85% identical to the amino acid sequence of SEQ ID NO: 131. In certain embodiments, the ST2 domain comprises an amino acid sequence that is no less than about 90% identical to the amino acid sequence of SEQ ID NO: 131. In certain embodiments, the ST2 domain comprises an amino acid sequence that is no less than about 91% identical to the amino acid sequence of SEQ ID NO: 131. In certain embodiments, the ST2 domain comprises an amino acid sequence that is no less than about 92% identical to the amino acid sequence of SEQ ID NO: 131. In certain embodiments, the ST2 domain comprises an amino acid sequence that is no less than about 93% identical to the amino acid sequence of SEQ ID NO: 131. In certain embodiments, the ST2 domain comprises an amino acid sequence that is no less than about 94% identical to the amino acid sequence of SEQ ID NO: 131. In certain embodiments, the ST2 domain comprises an amino acid sequence that is no less than about 95% identical to the amino acid sequence of SEQ ID NO: 131. In certain embodiments, the ST2 domain comprises an amino acid sequence that is no less than about 96% identical to the amino acid sequence of SEQ ID NO: 131. In certain embodiments, the ST2 domain comprises an amino acid sequence that is no less than about 97% identical to the amino acid sequence of SEQ ID NO: 131. In certain embodiments, the ST2 domain comprises an amino acid sequence that is no less than about 98% identical to the amino acid sequence of SEQ ID NO: 131. In certain embodiments, the ST2 domain comprises an amino acid sequence that is no less than about 99% identical to the amino acid sequence of SEQ ID NO: 131.

In certain embodiments, the ST2 domain further includes one or more additional substitutions, deletions, and/or insertions.

In one embodiment, provided herein is fusion protein A1: comprising an amino acid sequence of SEQ ID NO: 132; or fusion protein A2: comprising an amino acid sequence of SEQ ID NO: 133.

In another embodiment, provided herein is fusion protein A3: comprising an amino acid sequence of SEQ ID NO: 134; or fusion protein A4: comprising an amino acid sequence of SEQ ID NO: 135.

In yet another embodiment, provided herein is fusion protein A5: comprising an amino acid sequence of SEQ ID NO: 136; or fusion protein A6: comprising an amino acid sequence of SEQ ID NO: 137.

In yet another embodiment, provided herein is:
fusion protein A7: comprising an amino acid sequence of SEQ ID NO: 138;
fusion protein A8: comprising an amino acid sequence of SEQ ID NO: 139;
fusion protein A9: comprising an amino acid sequence of SEQ ID NO: 140;
fusion protein A10: comprising amino acid sequences of SEQ ID NOs: 141 and 143;
fusion protein A11: comprising amino acid sequences of SEQ ID NOs: 142 and 143;
fusion protein A12: comprising amino acid sequences of SEQ ID NOs: 144 and 145;
fusion protein A13: comprising amino acid sequences of SEQ ID NOs: 146 and 148;
fusion protein A14: comprising amino acid sequences of SEQ ID NOs: 147 and 148;
fusion protein A15: comprising an amino acid sequence of SEQ ID NO: 149;
fusion protein A16: comprising an amino acid sequence of SEQ ID NO: 150;
fusion protein A17: comprising amino acid sequences of SEQ ID NOs: 151, 153, and 159;
fusion protein A18: comprising amino acid sequences of SEQ ID NOs: 152, 153, and 159;
fusion protein A19: comprising amino acid sequences of SEQ ID NOs: 154, 156, and 159;
fusion protein A20: comprising amino acid sequences of SEQ ID NOs: 155, 156, and 159;
fusion protein A21: comprising amino acid sequences of SEQ ID NOs: 157 and 159;
fusion protein A22: comprising amino acid sequences of SEQ ID NOs: 158 and 159;
fusion protein A23: comprising amino acid sequences of SEQ ID NOs: 160 and 162;
fusion protein A24: comprising amino acid sequences of SEQ ID NOs: 161 and 162;
fusion protein A25: comprising amino acid sequences of SEQ ID NOs: 163 and 164;
fusion protein A26: comprising amino acid sequences of SEQ ID NOs: 165 and 166; or fusion protein A27: comprising an amino acid sequence of SEQ ID NO: 167.

### Fusion Protein Complexes

In one embodiment, provided herein is a fusion protein complex comprising an IL-33 fusion protein provided herein and an ST2; wherein each IL-33 domain in the IL-33 fusion protein is complexed with one ST2.

In one embodiment, the fusion protein complex provided herein comprises an IL-33 fusion protein and an ST2; wherein the IL-33 fusion protein comprises an IL-33 domain and an anti-HSA V_{H}H sdAb; wherein the C-terminus of the anti-HSA V_{H}H sdAb is connected to the N-terminus of the IL-33 domain directly or via a peptide linker; and wherein each IL-33 domain in the IL-33 fusion protein is complexed with one ST2.

In another embodiment, the fusion protein complex provided herein comprises an IL-33 fusion protein and an ST2; wherein the IL-33 fusion protein comprises an IL-33 domain and an anti-HSA V_{H}H sdAb; wherein the C-terminus of the anti-HSA V_{H}H sdAb is connected to the N-terminus of the IL-33 domain via a peptide linker; and wherein each IL-33 domain in the IL-33 fusion protein is complexed with one ST2.

In yet another embodiment, the fusion protein complex provided herein comprises an IL-33 fusion protein and two ST2; wherein the IL-33 fusion protein comprises first and second IL-33 domains and an Fc domain comprising first and second peptide chains; wherein the C-terminus of the first peptide chain of the Fc domain is connected to the N-terminus of the first IL-33 domain directly or via a peptide linker, and the C-terminus of the second peptide chain of the Fc domain is connected to the N-terminus of the second IL-33 domain directly or via a peptide linker; and wherein each IL-33 domain in the IL-33 fusion protein is complexed with one ST2.

In another embodiment, provided herein is a fusion protein complex comprising an ST2 fusion protein provided herein and an IL-33; wherein each ST2 domain in the ST2 fusion protein is complexed with one IL-33.

In one embodiment, the fusion protein complex provided herein comprises an ST2 fusion protein and an IL-33; wherein the ST2 fusion protein comprises an ST2 domain and an anti-HSA V_{H}H sdAb; wherein the C-terminus of the ST2 domain is connected to the N-terminus of the anti-HSA V_{H}H sdAb directly or via a peptide linker; and wherein each ST2 domain in the ST2 fusion protein is complexed with one IL-33.

In another embodiment, the fusion protein complex provided herein comprises an ST2 fusion protein and an IL-33; wherein the ST2 fusion protein comprises an ST2 domain and an anti-HSA V_{H}H sdAb; wherein the C-terminus of the ST2 domain is connected to the N-terminus of the anti-HSA V_{H}H sdAb directly; and wherein each ST2 domain in the ST2 fusion protein is complexed with one IL-33.

In yet another embodiment, the fusion protein complex provided herein comprises an ST2 fusion protein and an IL-33; wherein the ST2 fusion protein comprises an ST2 domain and an anti-HSA V_{H}H sdAb; wherein the C-terminus of the ST2 domain is connected to the N-terminus of the anti-HSA V_{H}H sdAb via a peptide linker; and wherein each ST2 domain in the ST2 fusion protein is complexed with one IL-33.

In yet another embodiment, the fusion protein complex provided herein comprises an ST2 fusion protein and two IL-33; wherein the ST2 fusion protein comprises first and second ST2 domains and an Fc domain comprising first and second peptide chains; wherein the C-terminus of the first ST2 domain is connected to the N-terminus of the first peptide chain of the Fc domain directly or via a peptide linker; and the C-terminus of the second ST2 domain is connected to the N-terminus of the second peptide chain of the Fc domain directly or via a peptide linker; and wherein each ST2 domain in the ST2 fusion protein is complexed with one IL-33.

In yet another embodiment, the fusion protein complex provided herein comprises an ST2 fusion protein and two IL-33; wherein the ST2 fusion protein comprises first and second ST2 domains and an Fc domain comprising first and second peptide chains; wherein the C-terminus of the first ST2 domain is connected to the N-terminus of the first peptide chain of the Fc domain directly; and the C-terminus of the second ST2 domain is connected to the N-terminus of the second peptide chain of the Fc domain directly; and wherein each ST2 domain in the ST2 fusion protein is complexed with one IL-33.

In yet another embodiment, the fusion protein complex provided herein comprises an ST2 fusion protein and two IL-33; wherein the ST2 fusion protein comprises first and second ST2 domains and an Fc domain comprising first and second peptide chains; wherein the C-terminus of the first peptide chain of the Fc domain is connected to the N-terminus of the first ST2 domain directly or via a peptide linker; and the C-terminus of the second peptide chain of the Fc domain is connected to the N-terminus of the second ST2 domain directly or via a peptide linker; and wherein each ST2 domain in the ST2 fusion protein is complexed with one IL-33.

In still another embodiment, the fusion protein complex provided herein comprises an ST2 fusion protein and two IL-33; wherein the ST2 fusion protein comprises first and second ST2 domains and an Fc domain comprising first and second peptide chains; wherein the C-terminus of the first peptide chain of the Fc domain is connected to the N-terminus of the first ST2 domain via a peptide linker; and the C-terminus of the second peptide chain of the Fc domain is connected to the N-terminus of the second ST2 domain via a peptide linker; and wherein each ST2 domain in the ST2 fusion protein is complexed with one IL-33.

In one embodiment, provided herein is:
fusion protein complex C1: comprising (i) a fusion protein comprising an amino acid sequence of SEQ ID NO: 132 and (ii) a free ST2 of SEQ ID NO: 130;
fusion protein complex C2: comprising (i) a fusion protein comprising an amino acid sequence of SEQ ID NO: 132 and (ii) a free ST2 of SEQ ID NO: 131;
fusion protein complex C3: comprising (i) a fusion protein comprising an amino acid sequence of SEQ ID NO: 133 and (ii) a free ST2 of SEQ ID NO: 130;
fusion protein complex C4: comprising (i) a fusion protein comprising an amino acid sequence of SEQ ID NO: 133 and (ii) a free ST2 of SEQ ID NO: 131;
fusion protein complex C5: comprising an amino acid sequence of SEQ ID NO: 138; and (ii) a free ST2 of SEQ ID NO: 130;
fusion protein complex C6: comprising an amino acid sequence of SEQ ID NO: 138; and (ii) a free ST2 of SEQ ID NO: 131;
fusion protein complex C7: comprising an amino acid sequence of SEQ ID NO: 139; and (ii) a free ST2 of SEQ ID NO: 130;
fusion protein complex C8: comprising an amino acid sequence of SEQ ID NO: 139; and (ii) a free ST2 of SEQ ID NO: 131;
fusion protein complex C9: comprising an amino acid sequence of SEQ ID NO: 140; and (ii) a free IL-33 of SEQ ID NO: 126;
fusion protein complex C10: comprising an amino acid sequence of SEQ ID NO: 140; and (ii) a free IL-33 of SEQ ID NO: 127;
fusion protein complex C11: comprising an amino acid sequence of SEQ ID NO: 140; and (ii) a free IL-33 of SEQ ID NO: 128; or
fusion protein complex C12: comprising an amino acid sequence of SEQ ID NO: 140; and (ii) a free IL-33 of SEQ ID NO: 129.

In one embodiment, each IL-33 domain in the fusion protein complex provided herein is complexed with one free ST2. In another embodiment, each ST2 domain in the fusion protein complex provided herein is complexed with one free IL-33.

### Pharmaceutical Compositions

In one embodiment, provided herein is a pharmaceutical composition comprising a fusion protein or fusion protein complex provided herein and a pharmaceutically acceptable excipient.

In one embodiment, the pharmaceutical composition is formulated as single dosage form.

In one embodiment, the pharmaceutical composition provided herein is a solid formulation. In another embodiment, the pharmaceutical composition provided herein is a lyophilized solid formulation. In yet another embodiment, the pharmaceutical composition provided herein is a solution. In still another embodiment, the pharmaceutical composition provided herein is an aqueous solution.

In one embodiment, the pharmaceutical composition provided herein is formulated in a dosage form for parenteral administration. In another embodiment, the pharmaceutical composition provided herein is formulated in a dosage form for intravenous administration. In yet another embodiment, the pharmaceutical composition provided herein is formulated in a dosage form for intradermal administration. In yet another embodiment, the pharmaceutical composition provided herein is formulated in a dosage form for intramuscular administration. In still another embodiment, the pharmaceutical composition provided herein is formulated in a dosage form for subcutaneous administration.

### Methods of Use

In one embodiment, provided herein is a method for treating, preventing, or ameliorating one or more symptoms of an IL-33-mediated disorder, disease, or condition in a subject, comprising administering to the subject in need thereof a therapeutically effective amount of a fusion protein or fusion protein complex provided herein.

In one embodiment, the IL-33-mediated disorder, disease, or condition is a proliferative disease.

In another embodiment, provided herein is a method for treating, preventing, or ameliorating one or more symptoms of a proliferative disease in a subject, comprising administering to the subject in need thereof a therapeutically effective amount of a fusion protein or fusion protein complex provided herein.

In certain embodiments, the proliferative disease is cancer. In certain embodiments, the cancer is melanoma.

In certain embodiments, the cancer is refractory and/or relapsed. In certain embodiments, the cancer is refractory. In certain embodiments, the cancer is relapsed. In certain embodiments, the cancer is metastatic. In certain embodiments, the cancer is unresectable. In certain embodiments, the cancer is metastatic.

In certain embodiments, the cancer is drug-resistant. In certain embodiment, the cancer is multidrug-resistant. In certain embodiments, the cancer is resistant to a chemotherapy. In certain embodiments, the cancer is resistant to an immunotherapy. In certain embodiments, the cancer is resistant to a standard therapy for the cancer.

In certain embodiments, the therapeutically effective amount of a fusion protein or fusion protein complex provided herein is ranging from about 0.001 to 100 mg per kg subject body weight per day (mg/kg per day), from about 0.01 to about 75 mg/kg per day, from about 0.1 to about 50 mg/kg per day, from about 0.5 to about 25 mg/kg per day, or from about 1 to about 20 mg/kg per day, which can be administered in single or multiple doses. In certain embodiments, the therapeutically effective amount of a fusion protein or fusion protein complex provided herein is ranging from about 0.005 to about 0.05 mg/kg per day. In certain embodiments, the therapeutically effective amount of a fusion protein or fusion protein complex provided herein is ranging from about 0.05 to about 0.5 mg/kg per day. In certain embodiments, the therapeutically effective amount of a fusion protein or fusion protein complex provided herein is ranging from about 0.5 to about 5.0 mg/kg per day. In certain embodiments, the therapeutically effective amount of a fusion protein or fusion protein complex provided herein is ranging from about 1 to about 15 mg/kg per day. In certain embodiments, the therapeutically effective amount of a fusion protein or fusion protein complex provided herein is ranging from about 1 to about 20 mg/kg per day. In certain embodiments, the therapeutically effective amount of a fusion protein or fusion protein complex provided herein is ranging from about 1 to about 50 mg/kg per day.

In certain embodiments, the subject is a mammal. In certain embodiments, the subject is a human.

In one embodiment, provided herein is a method of inhibiting the growth of a cell, comprising contacting the cell with an effective amount of a fusion protein or fusion protein complex provided herein.

In certain embodiments, the cell is a cancerous cell. In certain embodiments, the cell is a human cell. In certain embodiments, the cell is a human cancerous cell.

The disclosure will be further understood by the following non-limiting examples.

### EXAMPLES

### Example 1

### Cloning, Expression, and Purification of Fusion Proteins

The amino acid sequences of human IL-33 (hIL-33) and human ST2 (hST2) were obtained from UNIPROT (IL-33:95-270 aa; ST2: 19-328 aa). The amino acid sequences of a human anti-PD1-L1 antibody (VH & VL) were obtained from Therapeutics Antibody Database (TABS). The amino acid sequence of an anti-HSA was one disclosed in WO 2019/246004 A1 or WO 2020/172528 A1, the disclosure of each of which is incorporated herein by reference in its entirety.

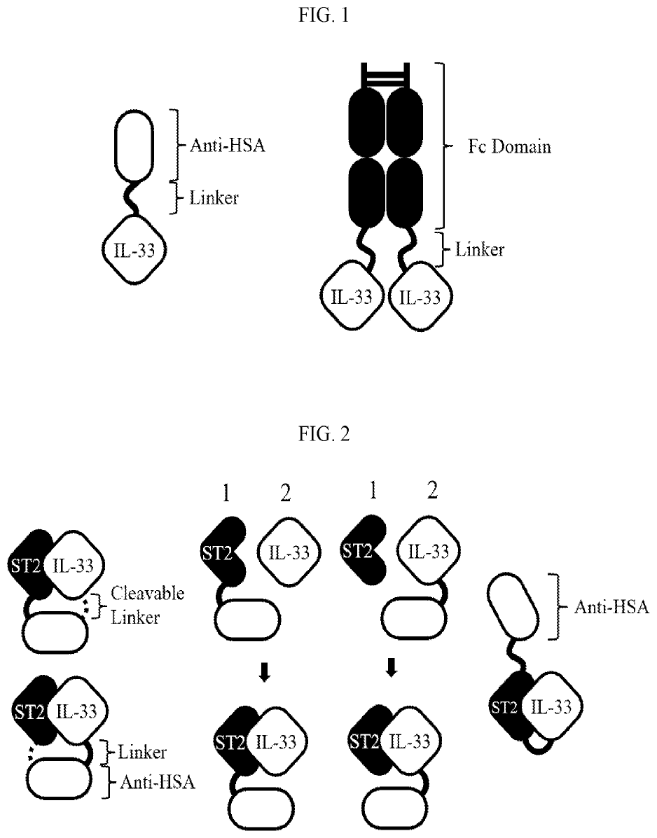

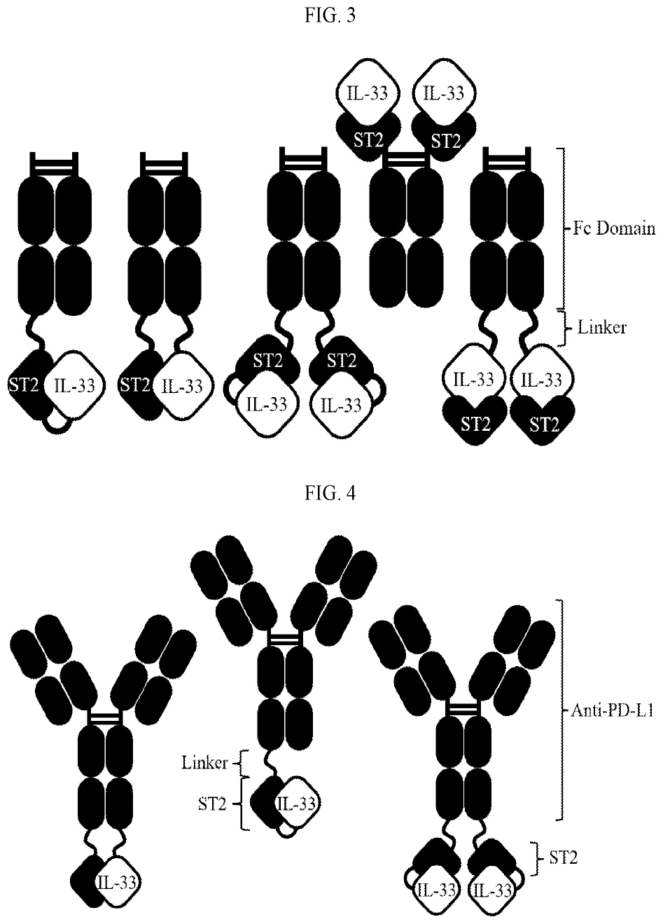

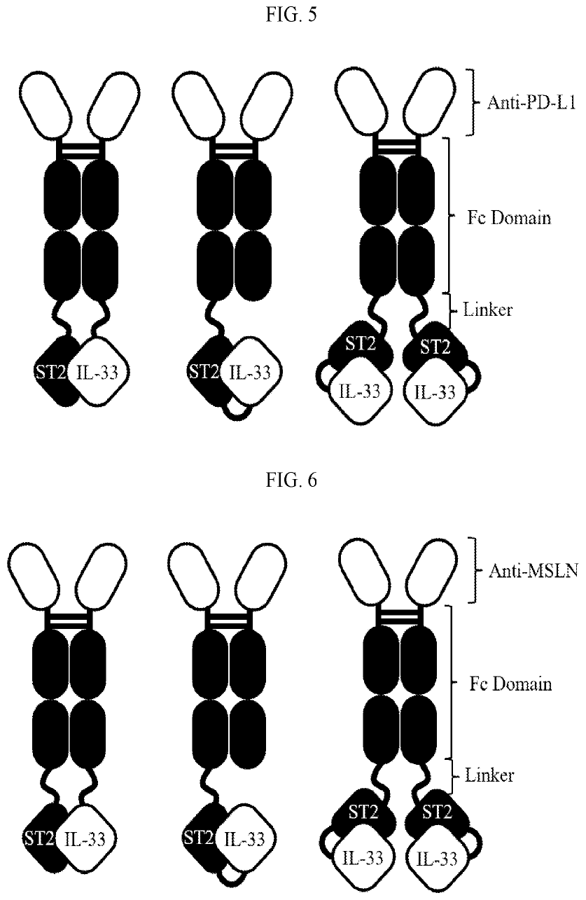

Certain configurations of a fusion protein comprising (i) an IL-33 domain and an anti-HSA or (ii) two IL-33 domains and an Fc domain are illustrated in FIG. 1. Certain configurations of a fusion protein comprising (i) an IL-33 domain, an ST2 domain, anti-HSA domain, and a cleavable peptide linker; (ii) an IL-33 domain or an ST2 domain, and anti-HSA domain, wherein the IL-33 domain of the fusion protein forms an IL-33/ST2 complex with a free ST2 or the ST2 domain of the fusion protein forms an IL-33/ST2 complex with a free IL-33; or (iii) an IL-33 domain, an ST2 domain, anti-HSA domain, and non-cleavable peptide linkers are illustrated in FIG. 2. Certain configurations of a fusion protein comprising (i) an IL-33 domain, an ST2 domain, and an Fc domain; (ii) an IL-33 domain, an ST2 domain, and an Fc domain; (iii) two IL-33 domains, two ST2 domains, and an Fc domain; (iv) two ST2 domains and an Fc domain, wherein each ST2 domain of the fusion protein is complexed with one free IL-33; or (iv) two IL-33 domains and an Fc domain, wherein each IL-33 domain of the fusion protein is complexed with one free ST2 are illustrated in FIG. 3. Certain configurations of a fusion protein comprising (i) an IL-33 domain, an ST2 domain, and an intact anti-PD-L1 antibody; (ii) an IL-33 domain, an ST2 domain, and an intact anti-PD-L1 antibody; or (iii) two IL-33 domains, two ST2 domains, and an anti-PD-L1 antibody are illustrated in FIG. 4. Certain configurations of a fusion protein comprising (i) an IL-33 domain, an ST2 domain, two anti-PD-L1 V_{H}H sdAbs, and an Fc domain; (ii) an IL-33 domain, an ST2 domain, two anti-PD-L1 V_{H}H sdAbs, and an Fc domain; and (iii) two IL-33 domains, two ST2 domain, two anti-PD-L1 V_{H}H sdAbs, and an Fc domain are illustrated in FIG. 5. Certain configurations of a fusion protein comprising (i) an IL-33 domain, an ST2 domain, two anti-MSLN V_{H}H sdAbs, and an Fc domain; (ii) an IL-33 domain, an ST2 domain, two anti-MSLN V_{H}H sdAbs, and an Fc domain; and (iii) two IL-33 domains, two ST2 domain, two anti-MSLN V_{H}H sdAbs, and an Fc domain are illustrated in FIG. 6.

For an IL-33/ST2-anti-PD-L1 fusion protein, the deoxyoligonucleotide (DNA) sequences encoding the human anti-PD1-L1 antibody (VH & VL), hIL-22 polypeptide, and ST2 polypeptide, respectively, were codon optimized for CHO cell expression. The DNA sequences encoding the anti-PD1-L1 antibody, hIL-33 polypeptide, ST2 polypeptide, and peptide linkers, respectively, were seamlessly assembled together by homology assembly cloning with a commercially available kit according to the design of the IL-33/ST2-anti-PD-L1 fusion protein. The oligonucleotides of the fusion protein were inserted into a UCOE® expression vector CET1019-AS-Puro for CHO cell expression.

After 8 days, the CHO cells were harvested and the fusion protein produced was purified by affinity chromatography using MABSELECT™ SURE™ resin. The fusion protein was eluted with a low pH elution buffer. The eluted fusion protein was buffer exchanged into a sodium phosphate buffer (20 mM, pH 6.0) prior to ion exchange chromatography.

The fusion protein was further purified by ion exchange chromatography on HITRAP™ CAPTO™ S IMPACT. The fusion protein was loaded onto the ion exchange column at the loading capacity no more than 20 mg protein/mL resin. The fusion protein was eluted with a linear salt gradient from 0 to 30% of 20 mM sodium phosphate with 1 M sodium chloride at pH 6.0 for 20 column volumes (CV). The fusion protein was eluted at conductivity around 10-12 mS/cm. The collected fractions were pooled and buffer exchanged into 20 mM sodium phosphate at pH 6.0 for storage.

### Example 2

### Stability of Fusion Proteins

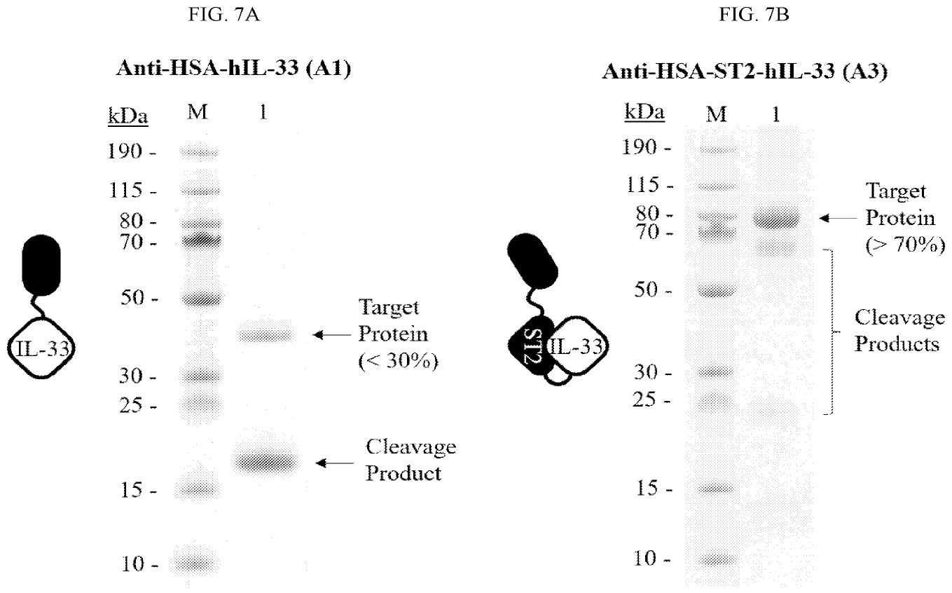

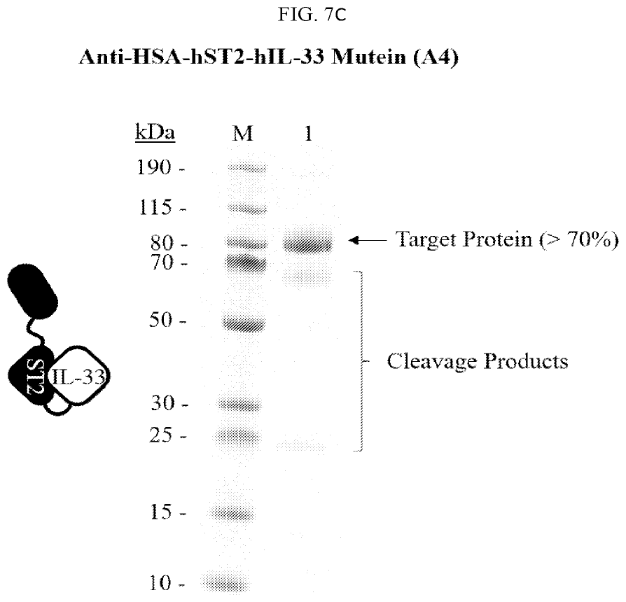

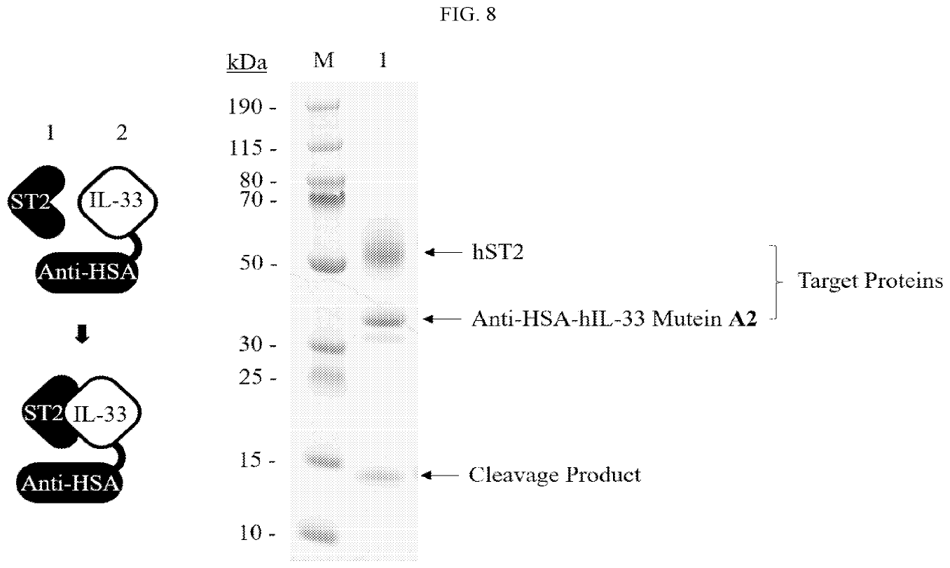

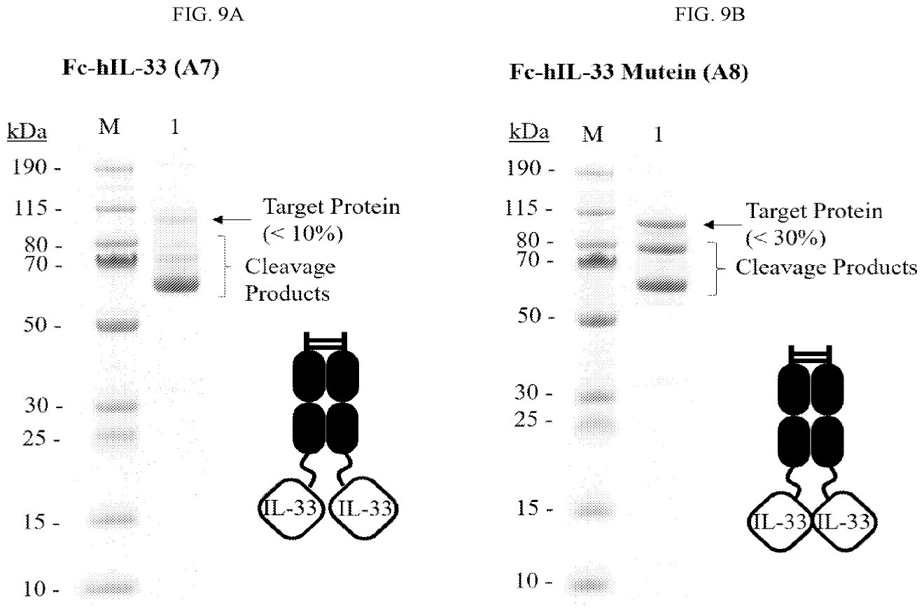

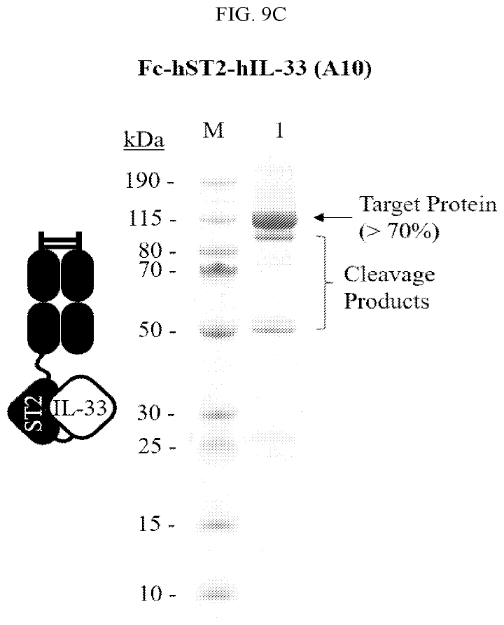

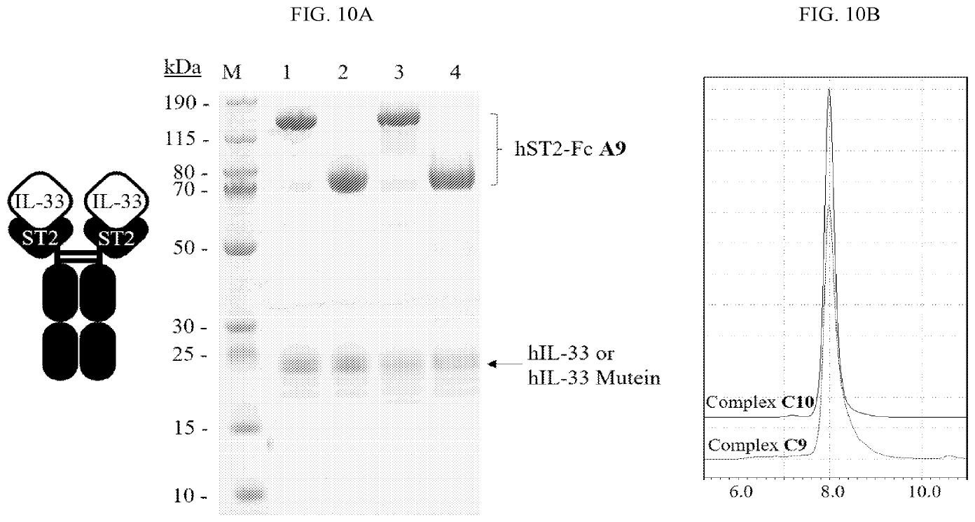

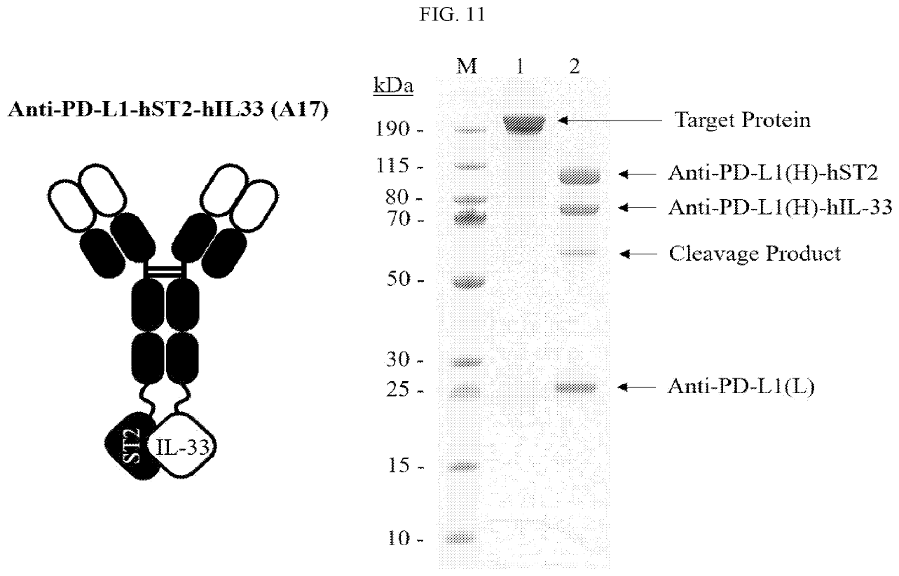

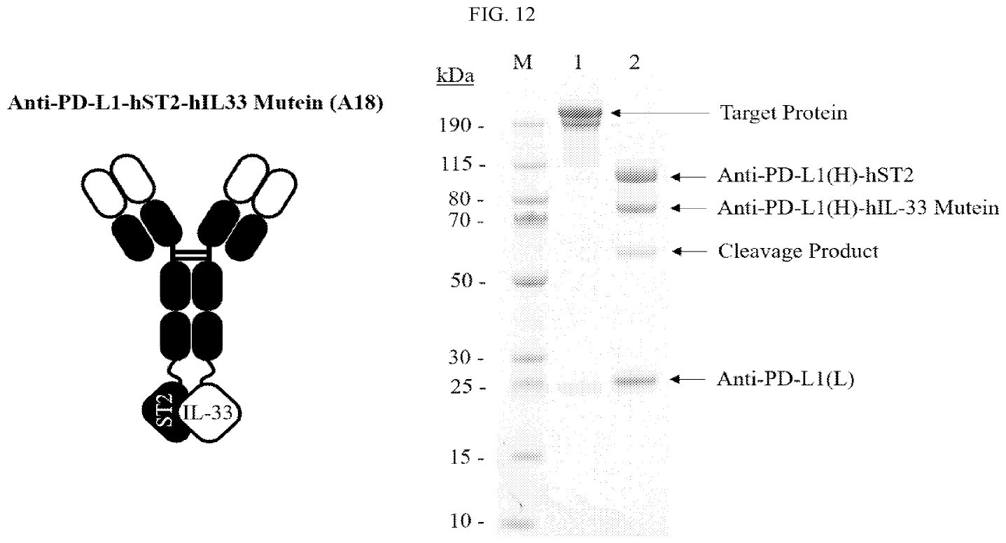

When hIL-33 was expressed as a fusion protein without hST2 in EXPICHO™ cells, a significant amount of the fusion protein eluted from protein A affinity column did not contain intact hIL-33. To protect hIL-33 from proteolytic cleavage, hIL-33 receptor, hST2, was introduced into the fusion protein. As shown in Table 1, in the presence of hST2, the cleavage of IL-33 was significantly reduced. The percentage of the intact protein was calculated based on the SDS-PAGE and peak profile of size exclusion chromatography. The SDS PAGE and SEC characterization of certain fusion proteins and fusion protein complexes is shown in FIG. 7A to FIG. 12, where the SDS gels were stained with INSTANT BLUE™.

TABLE 1
hIL-33 Stability

| | | Percentage of |
|---|---|---|
| hIL-33 Fusion Protein | SEQ ID NO: | Intact Protein |
| Anti-HSA-hIL-33 A1 | 136 | 5-30 |
| Anti-HSA-hIL-33 mutein A2 | 137 | 5-40 |
| hIgG1 Fc-hIL-33 A7 | 142 | 5-20 |
| hIgG1 Fc-hIL-33 mutein A8 | 143 | 5-40 |
| Anti-hHSA-hST2-IL-33 A3 | 138 | >70 |
| Anti-HSA-hST2-hIL-33 mutein A4 | 139 | >70 |
| hIgG1 Fc-hST2-hIL-33 A10 | 145 + 147 | >70 |
| Anti-PD-L1-hST2-hIL-33 A18 | 155 + 157 + 163 | >70 |

### Example 3

### Antitumor Activity of Fusion Proteins in a Xenograft Mouse Model for Melanoma

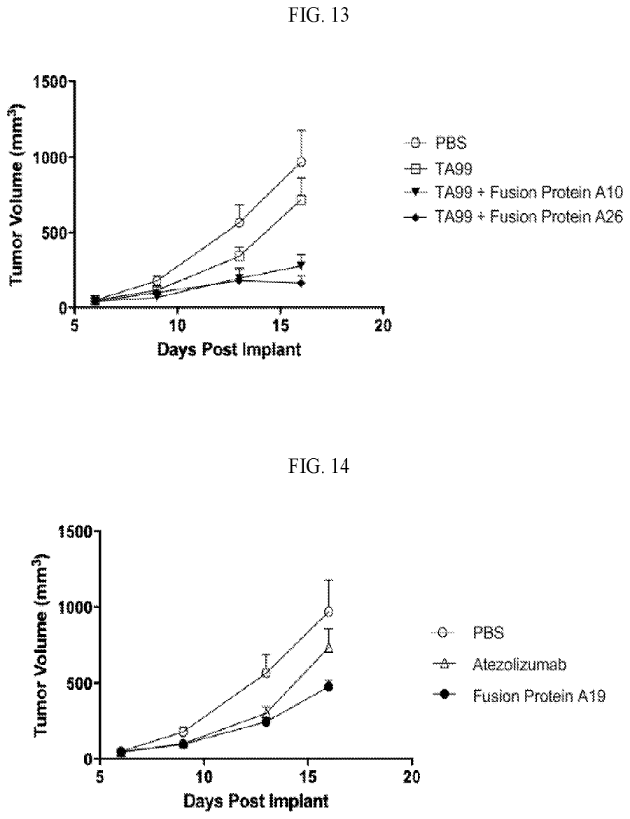

B16F10 cells were cultured and maintained in DMEM media supplemented with 10% fetal bovine serum, GLUTAMAX™, non-essential amino acids (NEAA), sodium pyruvate, and penicillin/streptomycin. The cells were trypsinized, washed with the media, and counted. The cells were diluted with PBS and 5×10⁵ cells in PBS (50 μL) were injected subcutaneously into anesthetized C57BL/6 mice using an 18-gauge needle. A stock solution of a fusion protein was diluted in PBS on the day of dosing and the mice are dosed intraperitoneally with PBS (control), anti-TRP1 antibody TA99 (100 μg), anti-PD-L1 antibody atezolizumab (25 μg), IL-33/ST2/anti-PD-L1 fusion protein A19 (35 μg), a combination of TA99 (100 μg) and IL-33/ST2/anti-PD-L1 fusion protein A10 (19 μg), or a combination of TA99 (100 μg) and IL-33/ST2/anti-MSLN fusion protein fusion protein A26 (23 μg) in PBS (100 μL) once or twice a week for two weeks. Tumor sizes (length (L) and width (W)) are measured twice per week using a digital caliper, and the tumor volume is calculated (L×W×W)/2. The results are summarized in FIGS. 13 and 14.

### Example 4

### IL-33 Signaling Assay

In vitro potency of a fusion protein is measured by quantifying phosphorylation of NFkB in BxPC3 cells, which express IL-33 receptor IL-1RL1. BxPC3 cells are maintained in RPMI-1640 medium containing 10% fetal bovine serum and penicillin/streptomycin. BxPC3 cells (100,000) are stimulated with a fusion protein at predetermined concentrations for 30 min at 37° C., 5% CO₂ in Hanks balanced salt solution containing 10 mM HEPES. Phospho-NFkB is measured using a phospho-NFkB (Ser536) homogeneous time resolved fluorescence (HTRF) assay. The signal ratio of 665 nm/620 nm is multiplied by 1000, plotted, and fit using a dose response curve to calculate an EC₅₀ value.

Sequences described herein are provided in the sequence table below.

SEQUENCE TABLE

| SEQ ID NO: | Description | Amino Acid Sequence |
|---|---|---|
| 1 | Anti-HSA-1 CDR1 | GSTWSINT |
| | (IMGT) | |
| 2 | Anti-HSA-1 CDR2 | ISSGGST |
| | (IMGT) | |
| 3 | Anti-HSA-1 CDR3 | YAQSTWYPPS |
| | (IMGT) | |
| 4 | Anti-HSA-1 FR1 | EVQLVESGGGLVQPGGSLRLSCAAS |
| 5 | Anti-HSA-1 FR2 | LAWYRQAPGKQRDLVAR |
| 6 | Anti-HSA-1 FR3 | YYADSVKGRFTISRDNSKNTLYLQMNSLRAEDTAVYYC |
| 7 | Anti-HSA-1 FR4 | WGQGTLVTVSS |
| 8 | Anti-HSA-1 | EVQLVESGGGLVQPGGSLRLSCAASGSTWSINTLAWYRQ |
| | | APGKQRDLVARISSGGSTYYADSVKGRFTISRDNSKNTLY |
| | | LQMNSLRAEDTAVYYCYAQSTWYPPSWGQGTLVTVSS |
| 9 | Anti-HSA-2 CDR1 | GFAFRGFG |
| | (IMGT) | |
| 10 | Anti-HSA-2 CDR2 | INNGGSDT |
| | (IMGT) | |
| 11 | Anti-HSA-2 CDR3 | AIGGPGASP |
| | (IMGT) | |
| 12 | Anti-HSA-2 FR1 | QVQLVESGGGVVQPGGSLRLSCAAS |
| 13 | Anti-HSA-2 FR2 | MSWVRQAPGKGLEWVSS |
| 14 | Anti-HSA-2 FR4 | SGQGTQVTVSS |
| 15 | Anti-HSA-2 | QVQLVESGGGVVQPGGSLRLSCAASGFAFRGFGMSWVRQ |
| | | APGKGLEWVSSINNGGSDTYYADSVKGRFTISRDNSKNTL |
| | | YLQMNSLRAEDTAVYYCAIGGPGASPSGQGTQVTVSS |
| | GSG Linker | GSG |
| 16 | (GSG)₂ Linker | GSGGSG |
| 17 | (GSG)₃ Linker | GSGGSGGSG |
| 18 | (GSG)₄ Linker | GSGGSGGSGGSG |
| 19 | G3S Linker | GGGS |
| 20 | (G3S)₂ Linker | GGGSGGGS |
| 21 | (G3S)₃ Linker | GGGSGGGSGGGS |
| 22 | (G3S)₄ Linker | GGGSGGGSGGGSGGGS |
| 23 | G4S Linker | GGGGS |
| 24 | (G4S)₂ Linker | GGGGSGGGGS |
| 25 | (G4S)₃ Linker | GGGGSGGGGSGGGGS |
| 26 | (G4S)₄ Linker | GGGGSGGGGSGGGGSGGGGS |
| 27 | SGSG Linker | SGSG |
| 28 | S(GSG)₂ Linker | SGSGGSG |
| 29 | S(GSG)₃ Linker | SGSGGSGGSG |
| 30 | S(GSG)₄ Linker | SGSGGSGGSGGSG |
| 31 | SG3S Linker | SGGGS |
| 32 | S(G3S)₂ Linker | SGGGSGGGS |
| 33 | S(G3S)₃ Linker | SGGGSGGGSGGGS |
| 34 | S(G3S)₄ Linker | SGGGSGGGSGGGSGGGS |
| 35 | SG4S Linker | SGGGGS |
| 36 | S(G4S)₂ Linker | SGGGGSGGGGS |
| 37 | S(G4S)₃ Linker | SGGGGSGGGGSGGGGS |
| 38 | S(G4S)₄ Linker | SGGGGSGGGGSGGGGSGGGGS |
| 39 | EAAAK Linker | EAAAK |
| 40 | (EAAAK)₂ Linker | EAAAKEAAAK |
| 41 | (EAAAK)₃ Linker | EAAAKEAAAKEAAAK |
| 42 | (EAAAK)₄ Linker | EAAAKEAAAKEAAAKEAAAK |
| 43 | PAPAP Linker | PAPAP |
| 44 | (PAPAP)₂ Linker | PAPAPPAPAP |
| 45 | (PAPAP)₃ Linker | PAPAPPAPAPPAPAP |
| 46 | (PAPAP)₄ Linker | PAPAPPAPAPPAPAPPAPAP |
| 47 | VLVH Linker | IKRTVAAP |
| 48 | RAKPS Linker | RAKPS |
| 49 | (RAKPS)₂ Linker | RAKPSRAKPS |
| 50 | ASTKG Linker | ASTKG |
| 51 | (ASTKG)₂ Linker | ASTKGASTKG |
| 52 | AKTHT Linker | AKTHT |
| 53 | (AKTHT)₂ Linker | AKTHTAKTHT |
| 54 | Cleavable Linker | RRKRAPVKQTLNFDLLKLAGDVESNPGP |
| 55 | hIgG1 CH1-CH3 | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSRE |
| | | EMTKNQVSLTCLVKGFYPSDIAVEWESNGQPENNYKTTPP |
| | | VLDSDGSFFLYSKLTVDKSRWQQGNVFSCSVMHEALHNH |
| | | YTQKSLSLSPGK |
| 56 | hIgG1 CH1-CH3 | ASTKGPSVFPLAPSSKSTSGGTAALGCLVKDYFPEPVTVS |
| | (N297A) | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYASTYRVVSVLTVLHQDWLN |
| | | GKEYKCKVSNKALPAPIEKTISKAKGQPREPQVYTLPPSRE |
| | | EMTKNQVSLTCLVKGFYPSDIAVEWESNGQPENNYKTTPP |
| | | VLDSDGSFFLYSKLTVDKSRWQQGNVFSCSVMHEALHNH |
| | | YTQKSLSLSPGK |
| 57 | hIgG1 CH1-CH3 | ASTKGPSVFPLAPSSKSTSGGTAALGCLVKDYFPEPVTVS |
| | (LALA, N297A) | WNSGALTSGVHTFPAVLQSSGLYSLSSVVTVPSSSLGTQT |
| | | YICNVNHKPSNTKVDKKVEPKSCDKTHTCPPCPAPEAAGG |
| | | PSVFLFPPKPKDTLMISRTPEVTCVVVDVSHEDPEVKFNW |
| | | YVDGVEVHNAKTKPREEQYASTYRVVSVLTVLHQDWLN |
| | | GKEYKCKVSNKALPAPIEKTISKAKGQPREPQVYTLPPSRE |
| | | EMTKNQVSLTCLVKGFYPSDIAVEWESNGQPENNYKTTPP |
| | | VLDSDGSFFLYSKLTVDKSRWQQGNVFSCSVMHEALHNH |
| | | YTQKSLSLSPGK |
| 58 | hIgG1 CH1-CH3 | ASTKGPSVFPLAPSSKSTSGGTAALGCLVKDYFPEPVTVS |
| | (S354C, T366W) | WNSGALTSGVHTFPAVLQSSGLYSLSSVVTVPSSSLGTQT |
| | (knob) | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPCRE |
| | | EMTKNQVSLWCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| 59 | hIgG1 CH1-CH3 | ASTKGPSVFPLAPSSKSTSGGTAALGCLVKDYFPEPVTVS |
| | (Y349C, T366S, | WNSGALTSGVHTFPAVLQSSGLYSLSSVVTVPSSSLGTQT |
| | L368A, Y407V) | YICNVNHKPSNTKVDKKVEPKSCDKTHTCPPCPAPELLGG |
| | (hole) | PSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPAPIEKTISKAKGQPREPQVCTLPPSRE |
| | | EMTKNQVSLSCAVKGFYPSDIAVEWESNGQPENNYKTTPP |
| | | VLDSDGSFFLVSKLTVDKSRWQQGNVFSCSVMHEALHNH |
| | | YTQKSLSLSPGK |
| 60 | hIgG1 CH1-CH3 | ASTKGPSVFPLAPSSKSTSGGTAALGCLVKDYFPEPVTVS |
| | (S354C, T366W, | WNSGALTSGVHTFPAVLQSSGLYSLSSVVTVPSSSLGTQT |
| | N297A) (knob) | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYASTYRVVSVLTVLHQDWLN |
| | | GKEYKCKVSNKALPAPIEKTISKAKGQPREPQVYTLPPCRE |
| | | EMTKNQVSLWCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| 61 | hIgG1 CH1-CH3 | ASTKGPSVFPLAPSSKSTSGGTAALGCLVKDYFPEPVTVS |
| | (Y349C, T366S, | WNSGALTSGVHTFPAVLQSSGLYSLSSVVTVPSSSLGTQT |
| | L368A, Y407V, | YICNVNHKPSNTKVDKKVEPKSCDKTHTCPPCPAPELLGG |
| | N297A) (hole) | PSVFLFPPKPKDTLMISRTPEVTCVVVDVSHEDPEVKFNW |
| | | YVDGVEVHNAKTKPREEQYASTYRVVSVLTVLHQDWLN |
| | | GKEYKCKVSNKALPAPIEKTISKAKGQPREPQVCTLPPSRE |
| | | EMTKNQVSLSCAVKGFYPSDIAVEWESNGQPENNYKTTPP |
| | | VLDSDGSFFLVSKLTVDKSRWQQGNVFSCSVMHEALHNH |
| | | YTQKSLSLSPGK |
| 62 | hIgG1 CH1-CH3 | ASTKGPSVFPLAPSSKSTSGGTAALGCLVKDYFPEPVTVS |
| | (S354C, T366W, | WNSGALTSGVHTFPAVLQSSGLYSLSSVVTVPSSSLGTQT |
| | LALA, N297A) | YICNVNHKPSNTKVDKKVEPKSCDKTHTCPPCPAPEAAGG |
| | (knob) | PSVFLFPPKPKDTLMISRTPEVTCVVVDVSHEDPEVKFNW |
| | | YVDGVEVHNAKTKPREEQYASTYRVVSVLTVLHQDWLN |
| | | GKEYKCKVSNKALPAPIEKTISKAKGQPREPQVYTLPPCRE |
| | | EMTKNQVSLWCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| 63 | hIgG1 CH1-CH3 | ASTKGPSVFPLAPSSKSTSGGTAALGCLVKDYFPEPVTVS |
| | (Y349C, T366S, | WNSGALTSGVHTFPAVLQSSGLYSLSSVVTVPSSSLGTQT |
| | L368A, Y407V, | YICNVNHKPSNTKVDKKVEPKSCDKTHTCPPCPAPEAAGG |
| | LALA, N297A) | PSVFLFPPKPKDTLMISRTPEVTCVVVDVSHEDPEVKFNW |
| | (hole) | YVDGVEVHNAKTKPREEQYASTYRVVSVLTVLHQDWLN |
| | | GKEYKCKVSNKALPAPIEKTISKAKGQPREPQVCTLPPSRE |
| | | EMTKNQVSLSCAVKGFYPSDIAVEWESNGQPENNYKTTPP |
| | | VLDSDGSFFLVSKLTVDKSRWQQGNVFSCSVMHEALHNH |
| | | YTQKSLSLSPGK |
| 64 | hIgG4 CH1-CH3 | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPPCPAPEFLGGPSVFL |
| | | FPPKPKDTLMISRTPEVTCVVVDVSQEDPEVQFNWYVDG |
| | | VEVHNAKTKPREEQFNSTYRVVSVLTVLHQDWLNGKEY |
| | | KCKVSNKGLPSSIEKTISKAKGQPREPQVYTLPPSQEEMTK |
| | | NQVSLTCLVKGFYPSDIAVEWESNGQPENNYKTTPPVLDS |
| | | DGSFFLYSRLTVDKSRWQEGNVFSCSVMHEALHNHYTQK |
| | | SLSLSLGK |
| 65 | hIgG4 CH1-CH3 | ASTKGPSVFPLAPCSRSTSESTAALGCLVKDYFPEPVTVSW |
| | (T366W) (knob) | NSGALTSGVHTFPAVLQSSGLYSLSSVVTVPSSSLGTKTYT |
| | | CNVDHKPSNTKVDKRVESKYGPPCPPCPAPEFLGGPSVFL |
| | | FPPKPKDTLMISRTPEVTCVVVDVSQEDPEVQFNWYVDG |
| | | VEVHNAKTKPREEQFNSTYRVVSVLTVLHQDWLNGKEY |
| | | KCKVSNKGLPSSIEKTISKAKGQPREPQVYTLPPSQEEMTK |
| | | NQVSLWCLVKGFYPSDIAVEWESNGQPENNYKTTPPVLD |
| | | SDGSFFLYSRLTVDKSRWQEGNVFSCSVMHEALHNHYTQ |
| | | KSLSLSLGK |
| 66 | hIgG4 CH1-CH3 | ASTKGPSVFPLAPCSRSTSESTAALGCLVKDYFPEPVTVSW |
| | (T366W, T368A) | NSGALTSGVHTFPAVLQSSGLYSLSSVVTVPSSSLGTKTYT |
| | (hole) | CNVDHKPSNTKVDKRVESKYGPPCPPCPAPEFLGGPSVFL |
| | | FPPKPKDTLMISRTPEVTCVVVDVSQEDPEVQFNWYVDG |
| | | VEVHNAKTKPREEQFNSTYRVVSVLTVLHQDWLNGKEY |
| | | KCKVSNKGLPSSIEKTISKAKGQPREPQVYTLPPSQEEMTK |
| | | NQVSLSCAVKGFYPSDIAVEWESNGQPENNYKTTPPVLDS |
| | | DGSFFLVSRLTVDKSRWQEGNVFSCSVMHEALHNHYTQK |
| | | SLSLSLGK |
| 67 | Anti-hPD-L1-A | QRVSSSYLA |
| | CDRL1 | |
| | (IMGT) | |
| 68 | Anti-hPD-L1-A | DASSRAT |
| | CDRL2 | |
| | (IMGT) | |
| 69 | Anti-hPD-L1-A | QQYGSLPWT |
| | CDRL3 | |
| | (IMGT) | |
| 70 | Anti-hPD-L1-A | GFTFSRY |
| | CDRH1 | |
| | (IMGT) | |
| 71 | Anti-hPD-L1-A | KQDGSE |
| | CDRH2 | |
| | (IMGT) | |
| 72 | Anti-hPD-L1-A | EGGWFGELAFDY |
| | CDRH3 | |
| | (IMGT) | |
| 73 | Anti-hPD-L1-A | EIVLTQSPGTLSLSPGERATLSCRASQRVSSSYLAWYQQKP |
| | Light Chain | GQAPRLLIYDASSRATGIPDRESGSGSGTDFTLTISRLEPED |
| | Variable | FAVYYCQQYGSLPWTFGQGTKVEIK |
| 74 | Anti-hPD-L1-A | EVQLVESGGGLVQPGGSLRLSCAASGFTFSRYWMSWVRQ |
| | Heavy Chain | APGKGLEWVANIKQDGSEKYYVDSVKGRFTISRDNAKNS |
| | Variable | LYLQMNSLRAEDTAVYYCAREGGWFGELAFDYWGQGTL |
| | | VTVSS |
| 75 | Anti-hPD-L1-A | EIVLTQSPGTLSLSPGERATLSCRASQRVSSSYLAWYQQKP |
| | Light Chain | GQAPRLLIYDASSRATGIPDRFSGSGSGTDFTLTISRLEPED |
| | | FAVYYCQQYGSLPWTFGQGTKVEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| 76 | Anti-hPD-L1-A | EVQLVESGGGLVQPGGSLRLSCAASGFTFSRYWMSWVRQ |
| | Heavy Chain | APGKGLEWVANIKQDGSEKYYVDSVKGRFTISRDNAKNS |
| | | LYLQMNSLRAEDTAVYYCAREGGWFGELAFDYWGQGTL |
| | | VTVSSASTKGPSVFPLAPSSKSTSGGTAALGCLVKDYFPEP |
| | | VTVSWNSGALTSGVHTFPAVLQSSGLYSLSSVVTVPSSSL |
| | | GTQTYICNVNHKPSNTKVDKRVEPKSCDKTHTCPPCPAPE |
| | | FEGGPSVFLFPPKPKDTLMISRTPEVTCVVVDVSHEDPEVK |
| | | FNWYVDGVEVHNAKTKPREEQYNSTYRVVSVLTVLHQD |
| | | WLNGKEYKCKVSNKALPASIEKTISKAKGQPREPQVYTLP |
| | | PSREEMTKNQVSLTCLVKGFYPSDIAVEWESNGQPENNYK |
| | | TTPPVLDSDGSFFLYSKLTVDKSRWQQGNVFSCSVMHEAL |
| | | HNHYTQKSLSLSPGK |
| 77 | Anti-hPD-L1-B | QDVSTAVA |
| | CDRL1 | |
| | (IMGT) | |
| 78 | Anti-hPD-L1-B | SASFLYS |
| | CDRL2 | |
| | (IMGT) | |
| 79 | Anti-hPD-L1-B | QQYLYHPAT |
| | CDRL3 | |
| | (IMGT) | |
| 80 | Anti-hPD-L1-B | GFTFSDS |
| | CDRH1 | |
| | (IMGT) | |
| 81 | Anti-hPD-L1-B | SPYGGS |
| | CDRH2 | |
| | (IMGT) | |
| 82 | Anti-hPD-L1-B | RHWPGGFDY |
| | CDRH3 | |
| | (IMGT) | |
| 83 | Anti-hPD-L1-B | DIQMTQSPSSLSASVGDRVTITCRASQDVSTAVAWYQQKP |
| | Light Chain | GKAPKLLIYSASFLYSGVPSRFSGSGSGTDFTLTISSLQPED |
| | Variable | FATYYCQQYLYHPATFGQGTKVEIK |
| 84 | Anti-hPD-L1-B | EVQLVESGGGLVQPGGSLRLSCAASGFTFSDSWIHWVRQ |
| | Heavy Chain | APGKGLEWVAWISPYGGSTYYADSVKGRFTISADTSKNT |
| | Variable | AYLQMNSLRAEDTAVYYCARRHWPGGFDYWGQGTLVT |
| | | VSS |
| 85 | Anti-hPD-L1-B | DIQMTQSPSSLSASVGDRVTITCRASQDVSTAVAWYQQKP |
| | Light Chain | GKAPKLLIYSASFLYSGVPSRFSGSGSGTDFTLTISSLQPED |
| | | FATYYCQQYLYHPATFGQGTKVEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| 86 | Anti-hPD-L1-B | EVQLVESGGGLVQPGGSLRLSCAASGFTFSDSWIHWVRQ |
| | Heavy Chain | APGKGLEWVAWISPYGGSTYYADSVKGRFTISADTSKNT |
| | | AYLQMNSLRAEDTAVYYCARRHWPGGFDYWGQGTLVT |
| | | VSSASTKGPSVFPLAPSSKSTSGGTAALGCLVKDYFPEPVT |
| | | VSWNSGALTSGVHTFPAVLQSSGLYSLSSVVTVPSSSLGT |
| | | QTYICNVNHKPSNTKVDKKVEPKSCDKTHTCPPCPAPELL |
| | | GGPSVFLFPPKPKDTLMISRTPEVTCVVVDVSHEDPEVKEN |
| | | WYVDGVEVHNAKTKPREEQYASTYRVVSVLTVLHQDWL |
| | | NGKEYKCKVSNKALPAPIEKTISKAKGQPREPQVYTLPPSR |
| | | EEMTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| 87 | Anti-hPD-L1-C | SSDVGGYNYVS |
| | CDRL1 | |
| | (IMGT) | |
| 88 | Anti-hPD-L1-C | DVSNRPS |
| | CDRL2 | |
| | (IMGT) | |
| 89 | Anti-hPD-L1-C | SSYTSSSTRV |
| | CDRL3 | |
| | (IMGT) | |
| 90 | Anti-hPD-L1-C | GFTFSSY |
| | CDRH1 | |
| | (IMGT) | |
| 91 | Anti-hPD-L1-C | YPSGGI |
| | CDRH2 | |
| | (IMGT) | |
| 92 | Anti-hPD-L1-C | IKLGTVTTVDY |
| | CDRH3 | |
| | (IMGT) | |
| 93 | Anti-hPD-L1-C | QSALTQPASVSGSPGQSITISCTGTSSDVGGYNYVSWYQQ |
| | Light Chain | HPGKAPKLMIYDVSNRPSGVSNRFSGSKSGNTASLTISGLQ |
| | Variable | AEDEADYYCSSYTSSSTRVFGTGTKVTVL |
| 94 | Anti-hPD-L1-C | EVQLLESGGGLVQPGGSLRLSCAASGFTFSSYIMMWVRQ |
| | Heavy Chain | APGKGLEWVSSIYPSGGITFYADTVKGRFTISRDNSKNTLY |
| | Variable | LQMNSLRAEDTAVYYCARIKLGTVTTVDYWGQGTLVTVS |
| | | S |
| 95 | Anti-hPD-L1-C | QSALTQPASVSGSPGQSITISCTGTSSDVGGYNYVSWYQQ |
| | Light Chain | HPGKAPKLMIYDVSNRPSGVSNRFSGSKSGNTASLTISGLQ |
| | | AEDEADYYCSSYTSSSTRVFGTGTKVTVLGQPKANPTVTL |
| | | FPPSSEELQANKATLVCLISDFYPGAVTVAWKADGSPVKA |
| | | GVETTKPSKQSNNKYAASSYLSLTPEQWKSHRSYSCQVTH |
| | | EGSTVEKTVAPTECS |
| 96 | Anti-hPD-L1-C | EVQLLESGGGLVQPGGSLRLSCAASGFTFSSYIMMWVRQ |
| | Heavy Chain | APGKGLEWVSSIYPSGGITFYADTVKGRFTISRDNSKNTLY |
| | | LQMNSLRAEDTAVYYCARIKLGTVTTVDYWGQGTLVTVS |
| | | SASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSRD |
| | | ELTKNQVSLTCLVKGFYPSDIAVEWESNGQPENNYKTTPP |
| | | VLDSDGSFFLYSKLTVDKSRWQQGNVFSCSVMHEALHNH |
| | | YTQKSLSLSPGK |
| 97 | Anti-hPD-L1-D | ESVSIHGTHL |
| | CDRL1 | |
| | (IMGT) | |
| 98 | Anti-hPD-L1-D | AASNLES |
| | CDRL2 | |
| | (IMGT) | |
| 99 | Anti-hPD-L1-D | QQSFEDPLT |
| | CDRL3 | |
| | (IMGT) | |
| 100 | Anti-hPD-L1-D | GYTFTSYW |
| | CDRH1 | |
| | (IMGT) | |
| 101 | Anti-hPD-L1-D | IGPNSGFT |
| | CDRH2 | |
| | (IMGT) | |
| 102 | Anti-hPD-L1-D | ARGGSSYDYFDY |
| | CDRH3 | |
| | (IMGT) | |
| 103 | Anti-hPD-L1-D | DIVLTQSPASLAVSPGQRATITCRASESVSIHGTHLMHWYQ |
| | Light Chain | QKPGQPPKLLIYAASNLESGVPARFSGSGSGTDFTLTINPV |
| | Variable | EAEDTANYYCQQSFEDPLTFGQGTKLEIK |
| 104 | Anti-hPD-L1-D | QVQLVQSGAEVKKPGASVKVSCKASGYTFTSYWMHWVR |
| | Heavy Chain | QAPGQGLEWMGRIGPNSGFTSYNEKFKNRVTMTRDTSTS |
| | Variable | TVYMELSSLRSEDTAVYYCARGGSSYDYFDYWGQGTTVT |
| | | VSS |
| 105 | Anti-hPD-L1-D | DIVLTQSPASLAVSPGQRATITCRASESVSIHGTHLMHWYQ |
| | Light Chain | QKPGQPPKLLIYAASNLESGVPARFSGSGSGTDFTLTINPV |
| | | EAEDTANYYCQQSFEDPLTFGQGTKLEIKRTVAAPSVFIFP |
| | | PSDEQLKSGTASVVCLLNNFYPREAKVQWKVDNALQSGN |
| | | SQESVTEQDSKDSTYSLSSTLTLSKADYEKHKVYACEVTH |
| | | QGLSSPVTKSFNRGEC |
| 106 | Anti-hPD-L1-D | QVQLVQSGAEVKKPGASVKVSCKASGYTFTSYWMHWVR |
| | Heavy Chain | QAPGQGLEWMGRIGPNSGFTSYNEKFKNRVTMTRDTSTS |
| | | TVYMELSSLRSEDTAVYYCARGGSSYDYFDYWGQGTTVT |
| | | VSSASTKGPSVFPLAPCSRSTSESTAALGCLVKDYFPEPVT |
| | | VSWNSGALTSGVHTFPAVLQSSGLYSLSSVVTVPSSSLGT |
| | | KTYTCNVDHKPSNTKVDKRVESKYGPPCPPCPAPEAAGGP |
| | | SVFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPP |
| | | VLDSDGSFFLYSRLTVDKSRWQEGNVFSCSVMHEALHNH |
| | | YTQKSLSLSLGK |
| 107 | Anti-hPD-L1-E | YVLTQPPSVSVAPGQTARITCGGNNIGSKSVHWYQQKPGQ |
| | Light Chain | APVLVVYDDSDRPSGIPERFSGSNSGNTATLTISRVEAGDE |
| | | ADYYCQVWDSSSDHVVFGGGTKLTVLGQPKAAPSVTLFP |
| | | PSSEELQANKATLVCLISDFYPGAVTVAWKADSSPVKAGV |
| | | ETTTPSKQSNNKYAASSYLSLTPEQWKSHRSYSCQVTHEG |
| | | STVEKTVAPTECS |
| 108 | Anti-hPD-L1-E | EVQLLESGGGLVQPGGSLRLSCAASGFTFSSYAMSWVRQ |
| | Heavy Chain | APGKGLEWVSGISGSGGFTYYADSVKGRFTISRDNSKNTL |
| | | YLQMNSLRAEDTAVYYCAKPPRGYNYGPFDYWGQGTLV |
| | | TVSSASTKGPSVFPLAPCSRSTSESTAALGCLVKDYFPEPV |
| | | TVSWNSGALTSGVHTFPAVLQSSGLYSLSSVVTVPSSSLGT |
| | | KTYTCNVDHKPSNTKVDKRVESKYGPPCPPCPAPEFLGGP |
| | | SVFLFPPKPKDTLMISRTPEVTCVVVDVSQEDPEVQFNWY |
| | | VDGVEVHNAKTKPREEQFNSTYRVVSVLTVLHQDWLNG |
| | | KEYKCKVSNKGLPSSIEKTISKAKGQPREPQVYTLPPSQEE |
| | | MTKNQVSLTCLVKGFYPSDIAVEWESNGQPENNYKTTPP |
| | | VLDSDGSFFLYSRLTVDKSRWQEGNVFSCSVMHEALHNH |
| | | YTQKSLSLSLGK |
| 109 | Anti-hPD-L1-F | NFMLTQPHSVSESPGKTVTISCTRSSGSIDSNYVQWYQQRP |
| | Light Chain | GSAPTTVIYEDNQRPSGVPDRFSGSIDSSSNSASLTISGLKT |
| | | EDEADYYCQSYDSNNRHVIFGGGTKLTVLGQPKAAPSVT |
| | | LFPPSSEELQANKATLVCLISDFYPGAVTVAWKADSSPVK |
| | | AGVETTTPSKQSNNKYAASSYLSLTPEQWKSHRSYSCQVT |
| | | HEGSTVEKTVAPTECS |
| 110 | Anti-hPD-L1-F | EVQLVQSGAEVKKPGSSVKVSCKASGGTFSRSAISWVRQA |
| | Heavy Chain | PGQGLEWMGVIIPAFGEANYAQKFQGRVTITADESTSTAY |
| | | MELSSLRSEDTAVYYCARGRQMFGAGIDFWGQGTLVTVS |
| | | SASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSRD |
| | | ELTKNQVSLTCLVKGFYPSDIAVEWESNGQPENNYKTTPP |
| | | VLDSDGSFFLYSKLTVDKSRWQQGNVFSCSVMHEALHNH |
| | | YTQKSLSLSPGK |
| 111 | Anti-hPD-L1-G | QGQSGSGIALCPSHFCQLPQTGGGSSGGSGGSGGISSGLLS |
| | Light Chain | GRSDNHGGSDIQMTQSPSSLSASVGDRVTITCRASQSISSY |
| | | LNWYQQKPGKAPKLLIYAASSLQSGVPSRFSGSGSGTDFT |
| | | LTISSLQPEDFATYYCQQDNGYPSTFGGGTKVEIKRTVAAP |
| | | SVFIFPPSDEQLKSGTASVVCLLNNFYPREAKVQWKVDNA |
| | | LQSGNSQESVTEQDSKDSTYSLSSTLTLSKADYEKHKVYA |
| | | CEVTHQGLSSPVTKSFNRGEC |
| 112 | Anti-hPD-L1-G | EVQLLESGGGLVQPGGSLRLSCAASGFTFSSYAMSWVRQ |
| | Heavy Chain | APGKGLEWVSSIWRNGIVTVYADSVKGRFTISRDNSKNTL |
| | | YLQMNSLRAEDTAVYYCAKWSAAFDYWGQGTLVTVSSA |
| | | STKGPSVFPLAPCSRSTSESTAALGCLVKDYFPEPVTVSWN |
| | | SGALTSGVHTFPAVLQSSGLYSLSSVVTVPSSSLGTKTYTC |
| | | NVDHKPSNTKVDKRVESKYGPPCPPCPAPEFLGGPSVFLFP |
| | | PKPKDTLMISRTPEVTCVVVDVSQEDPEVQFNWYVDGVE |
| | | VHNAKTKPREEQFFNSTYRVVSVLTVLHQDWLNGKEYKC |
| | | KVSNKGLPSSIEKTISKAKGQPREPQVYTLPPSQEEMTKNQ |
| | | VSLTCLVKGFYPSDIAVEWESNGQPENNYKTTPPVLDSDG |
| | | SFFLYSRLTVDKSRWQEGNVFSCSVMHEALHNHYTQKSL |
| | | SLSLG |
| 113 | Anti-hPD-L1-H | GKMSSRRC |
| | V_{H}H CDR1 | |
| | (IMGT) | |
| 114 | Anti-hPD-L1-H | LLTTSGST |
| | V_{H}H CDR2 | |
| | (IMGT) | |
| 115 | Anti-hPD-L1-H | AADSFEDPTCTLVTSSGAFQY |
| | V_{H}H CDR3 | |
| | (IMGT) | |
| 116 | Anti-hPD-L1-H | QVQLVESGGGLVQPGGSLRLSCAASGKMSSRRCMAWFR |
| | V_{H}H | QAPGKERERVAKLLTTSGSTYLADSVKGRFTISRDNSKNT |
| | | VYLQMNSLRAEDTAVYYCAADSFEDPTCTLVTSSGAFQY |
| | | WGQGTLVTVSSEPKSSDKTHTCPPCPAPELLGGPSVFLFPP |
| | | KPKDTLMISRTPEVTCVVVAVSHEDPEVKFNWYVDGVEV |
| | | HNAKTKPREEQYNSTYRVVSVLTVLHQDWLNGKEYKCK |
| | | VSNKALPAGIEKTISKAKGQPREPQVYTLPPSRDELTKNQV |
| | | SLTCLVKGFYPSDIAVEWESNGQPENNYKTTPPVLDSDGS |
| | | FFLYSKLTVDKSRWQQGNVFSCSVMHEALHNHYTQKSLS |
| | | LSPGK |
| 117 | Anti-MSLN | GITFPVNA |
| | V_{H}H CDR1 | |
| | (IMGT) | |
| 118 | Anti-MSLN | ISAGGTT |
| | V_{H}H CDR2 | |
| | (IMGT) | |
| 119 | Anti-MSLN | YLQRRIGMLRDY |
| | V_{H}H CDR3 | |
| | (IMGT) | |
| 120 | Anti-MSLN FR1 | QVQLVESGGGLVQPGGSLRLSCAAS |
| 121 | Anti-MSLN FR2 | YGWYRQAPGKQRDLVAI |
| 122 | Anti-MSLN FR3 | NYADSVKGRFTISRDNSKNTLYLQMNSLRAEDTAVYYC |
| 123 | Anti-MSLN FR4 | WGQGTQVTVSS |
| 124 | Anti-MSLN | QVQLVESGGGLVQPGGSLRLSCAASGITFPVNAYGWYRQ |
| | V_{H}H | APGKQRDLVAIISAGGTTNYADSVKGRFTISRDNSKNTLY |
| | | LQMNSLRAEDTAVYYCYLQRRIGMLRDYWGQGTQVTVS |
| | | S |
| 125 | hIL-33 | MKPKMKYSTNKISTAKWKNTASKALCFKLGKSQQKAKE |
| | (full length) | VCPMYFMKLRSGLMIKKEACYFRRETTKRPSLKTGRKHK |
| | | RHLVLAACQQQSTVECFAFGISGVQKYTRALHDSSITGISPI |
| | | TEYLASLSTYNDQSITFALEDESYEIYVEDLKKDEKKDKVL |
| | | LSYYESQHPSNESGDGVDGKMLMVTLSPTKDFWLHANN |
| | | KEHSVELHKCEKPLPDQAFFVLHNMHSNCVSFECKTDPG |
| | | VFIGVKDNHLALIKVDSSENLCTENILFKLSET |
| 126 | hIL-33 | AFGISGVQKYTRALHDSSITGISPITEYLASLSTYNDQSITFA |
| | (95-270 aa) | LEDESYEIYVEDLKKDEKKDKVLLSYYESQHPSNESGDGV |
| | | DGKMLMVTLSPTKDFWLHANNKEHSVELHKCEKPLPDQ |
| | | AFFVLHNMHSNCVSFECKTDPGVFIGVKDNHLALIKVDSS |
| | | ENLCTENILFKLSET |
| 127 | hIL-33 mutein | AFGISGVQKYTRALHDSSITGISPITEYLASLSTYNDQSITFA |
| | (95-270 aa) | LEDESYEIYVEDLKKDEKKDKVLLSYYESQHPSNESGDGV |
| | (C208S, C227S, | DGKMLMVTLSPTKDFWLHANNKEHSVELHKSEKPLPDQA |
| | C232S, C259S) | FFVLHNMHSNSVSFESKTDPGVFIGVKDNHLALIKVDSSE |
| | | NLSTENILFKLSET |
| 128 | hIL-33 | SITGISPITEYLASLSTYNDQSITFALEDESYEIYVEDLKKDE |
| | (112-270 aa) | KKDKVLLSYYESQHPSNESGDGVDGKMLMVTLSPTKDF |
| | | WLHANNKEHSVELHKCEKPLPDQAFFVLHNMHSNCVSFE |
| | | CKTDPGVFIGVKDNHLALIKVDSSENLCTENILFKLSET |
| 129 | hIL-33 mutein | SITGISPITEYLASLSTYNDQSITFALEDESYEIYVEDLKKDE |
| | (112-270 aa) | KKDKVLLSYYESQHPSNESGDGVDGKMLMVTLSPTKDF |
| | (C208S, C227S, | WLHANNKEHSVELHKSEKPLPDQAFFVLHNMHSNSVSFE |
| | C232S, C259S) | SKTDPGVFIGVKDNHLALIKVDSSENLSTENILFKLSET |
| 130 | hST2 | KESKQSWGLENEALIVRCPRQGKPSYTVDWYYSQTNKSIP |
| | (19-328 aa) | TQERNRVFASGQLLKFLPAAVADSGIYTCIVRSPTFNRTGY |
| | | ANVTIYKKQSDCNVPDYLMYSTVSGSEKNSKIYCPTIDLY |
| | | NWTAPLEWFKNCQALQGSRYRAHKSFLVIDNVMTEDAG |
| | | DYTCKFIHNENGANYSVTATRSFTVKDEQGFSLFPVIGAPA |
| | | QNEIKEVEIGKNANLTCSACFGKGTQFLAAVLWQLNGTKI |
| | | TDFGEPRIQQEEGQNQSFSNGLACLDMVLRIADVKEEDLL |
| | | LQYDCLALNLHGLRRHTVRLSRK |
| 131 | hST2 | KFSKQSWGLENEALIVRCPRQGKPSYTVDWYYSQTNKSIP |
| | (19-197 aa) | TQERNRVFASGQLLKFLPAAVADSGIYTCIVRSPTFNRTGY |
| | | ANVTIYKKQSDCNVPDYLMYSTVSGSEKNSKIYCPTIDLY |
| | | NWTAPLEWFKNCQALQGSRYRAHKSFLVIDNVMTEDAG |
| | | DYTCKFIHNENGANYSVTAT |
| 132 | Anti-HSA-hIL-33 | EVQLVESGGGLVQPGGSLRLSCAASGSTWSINTLAWYRQ |
| | A1 | APGKQRDLVARISSGGSTYYADSVKGRFTISRDNSKNTLY |
| | | LQMNSLRAEDTAVYYCYAQSTWYPPSWGQGTLVTVSSAF |
| | | GISGVQKYTRALHDSSITGISPITEYLASLSTYNDQSITFALE |
| | | DESYEIYVEDLKKDEKKDKVLLSYYESQHPSNESGDGVD |
| | | GKMLMVTLSPTKDFWLHANNKEHSVELHKCEKPLPDQAF |
| | | FVLHNMHSNCVSFECKTDPGVFIGVKDNHLALIKVDSSEN |
| | | LCTENILFKLSET |
| 133 | Anti-HSA-hIL-33 | EVQLVESGGGLVQPGGSLRLSCAASGSTWSINTLAWYRQ |
| | mutein (C208S, | APGKQRDLVARISSGGSTYYADSVKGRFTISRDNSKNTLY |
| | C227S, C232S, | LQMNSLRAEDTAVYYCYAQSTWYPPSWGQGTLVTVSSAF |
| | C259S) | GISGVQKYTRALHDSSITGISPITEYLASLSTYNDQSITFALE |
| | A2 | DESYEIYVEDLKKDEKKDKVLLSYYESQHPSNESGDGVD |
| | | GKMLMVTLSPTKDFWLHANNKEHSVELHKSEKPLPDQAF |
| | | FVLHNMHSNSVSFESKTDPGVFIGVKDNHLALIKVDSSEN |
| | | LSTENILFKLSET |
| 134 | Anti-HSA-hST2- | EVQLVESGGGLVQPGGSLRLSCAASGSTWSINTLAWYRQ |
| | hIL-33 | APGKQRDLVARISSGGSTYYADSVKGRFTISRDNSKNTLY |
| | A3 | LQMNSLRAEDTAVYYCYAQSTWYPPSWGQGTLVTVSSG |
| | | GGGSGGGGSGGGGSKFSKQSWGLENEALIVRCPRQGKPS |
| | | YTVDWYYSQTNKSIPTQERNRVFASGQLLKFLPAAVADS |
| | | GIYTCIVRSPTFNRTGYANVTIYKKQSDCNVPDYLMYSTV |
| | | SGSEKNSKIYCPTIDLYNWTAPLEWFKNCQALQGSRYRAH |
| | | KSFLVIDNVMTEDAGDYTCKFIHNENGANYSVTATRSFTV |
| | | KDEQGFSLFPVIGAPAQNEIKEVEIGKNANLTCSACFGKGT |
| | | QFLAAVLWQLNGTKITDFGEPRIQQEEGQNQSFSNGLACL |
| | | DMVLRIADVKEEDLLLQYDCLALNLHGLRRHTVRLSRKG |
| | | GGSGGGSGGGSGGGSAFGISGVQKYTRALHDSSITGISPIT |
| | | EYLASLSTYNDQSITFALEDESYEIYVEDLKKDEKKDKVLL |
| | | SYYESQHPSNESGDGVDGKMLMVTLSPTKDFWLHANNK |
| | | EHSVELHKCEKPLPDQAFFVLHNMHSNCVSFECKTDPGVF |
| | | IGVKDNHLALIKVDSSENLCTENILFKLSET |
| 135 | Anti-HSA-hST2- | EVQLVESGGGLVQPGGSLRLSCAASGSTWSINTLAWYRQ |
| | hIL-33 mutein | APGKQRDLVARISSGGSTYYADSVKGRFTISRDNSKNTLY |
| | (C208S, C227S, | LQMNSLRAEDTAVYYCYAQSTWYPPSWGQGTLVTVSSG |
| | C232S, C259S) | GGGSGGGGSGGGGSKFSKQSWGLENEALIVRCPRQGKPS |
| | A4 | YTVDWYYSQTNKSIPTQERNRVFASGQLLKFLPAAVADS |
| | | GIYTCIVRSPTFNRTGYANVTIYKKQSDCNVPDYLMYSTV |
| | | SGSEKNSKIYCPTIDLYNWTAPLEWFKNCQALQGSRYRAH |
| | | KSFLVIDNVMTEDAGDYTCKFIHNENGANYSVTATRSFTV |
| | | KDEQGFSLFPVIGAPAQNEIKEVEIGKNANLTCSACFGKGT |
| | | QFLAAVLWQLNGTKITDFGEPRIQQEEGQNQSFSNGLACL |
| | | DMVLRIADVKEEDLLLQYDCLALNLHGLRRHTVRLSRKG |
| | | GGSGGGSGGGSGGGSAFGISGVQKYTRALHDSSITGISPIT |
| | | EYLASLSTYNDQSITFALEDESYEIYVEDLKKDEKKDKVLL |
| | | SYYESQHPSNESGDGVDGKMLMVTLSPTKDFWLHANNK |
| | | EHSVELHKSEKPLPDQAFFVLHNMHSNSVSFESKTDPGVFI |
| | | GVKDNHLALIKVDSSENLSTENILFKLSET |
| 136 | hST2-anti-HSA- | KFSKQSWGLENEALIVRCPRQGKPSYTVDWYYSQTNKSIP |
| | hIL-33 | TQERNRVFASGQLLKFLPAAVADSGIYTCIVRSPTENRTGY |
| | A5 | ANVTIYKKQSDCNVPDYLMYSTVSGSEKNSKIYCPTIDLY |
| | | NWTAPLEWFKNCQALQGSRYRAHKSFLVIDNVMTEDAG |
| | | DYTCKFIHNENGANYSVTATRSFTVKDEQGFSLFPVIGAPA |
| | | QNEIKEVEIGKNANLTCSACFGKGTQFLAAVLWQLNGTKI |
| | | TDFGEPRIQQEEGQNQSFSNGLACLDMVLRIADVKEEDLL |
| | | LQYDCLALNLHGLRRHTVRLSRKGGGGSGGGGSEVQLVE |
| | | SGGGLVQPGGSLRLSCAASGSTWSINTLAWYRQAPGKQR |
| | | DLVARISSGGSTYYADSVKGRFTISRDNSKNTLYLQMNSL |
| | | RAEDTAVYYCYAQSTWYPPSWGQGTLVTVSSRRKRAPV |
| | | KQTLNFDLLKLAGDVESNPGPAFGISGVQKYTRALHDSSIT |
| | | GISPITEYLASLSTYNDQSITFALEDESYEIYVEDLKKDEKK |
| | | DKVLLSYYESQHPSNESGDGVDGKMLMVTLSPTKDFWLH |
| | | ANNKEHSVELHKCEKPLPDQAFFVLHNMHSNCVSFECKT |
| | | DPGVFIGVKDNHLALIKVDSSENLCTENILFKLSET |
| 137 | hST2-anti-HSA- | KFSKQSWGLENEALIVRCPRQGKPSYTVDWYYSQTNKSIP |
| | hIL-33 mutein | TQERNRVFASGQLLKFLPAAVADSGIYTCIVRSPTFNRTGY |
| | (C208S, C227S, | ANVTIYKKQSDCNVPDYLMYSTVSGSEKNSKIYCPTIDLY |
| | C232S, C259S) | NWTAPLEWFKNCQALQGSRYRAHKSFLVIDNVMTEDAG |
| | A6 | DYTCKFIHNENGANYSVTATRSFTVKDEQGFSLFPVIGAPA |
| | | QNEIKEVEIGKNANLTCSACFGKGTQFLAAVLWQLNGTKI |
| | | TDFGEPRIQQEEGQNQSFSNGLACLDMVLRIADVKEEDLL |
| | | LQYDCLALNLHGLRRHTVRLSRKGGGGSGGGGSEVQLVE |
| | | SGGGLVQPGGSLRLSCAASGSTWSINTLAWYRQAPGKQR |
| | | DLVARISSGGSTYYADSVKGRFTISRDNSKNTLYLQMNSL |
| | | RAEDTAVYYCYAQSTWYPPSWGQGTLVTVSSRRKRAPV |
| | | KQTLNFDLLKLAGDVESNPGPAFGISGVQKYTRALHDSSIT |
| | | GISPITEYLASLSTYNDQSITFALEDESYEIYVEDLKKDEKK |
| | | DKVLLSYYESQHPSNESGDGVDGKMLMVTLSPTKDFWLH |
| | | ANNKEHSVELHKSEKPLPDQAFFVLHNMHSNSVSFESKTD |
| | | PGVFIGVKDNHLALIKVDSSENLSTENILFKLSET |
| 138 | hIgG1 Fc-hIL-33 | DKTHTCPPCPAPELLGGPSVFLFPPKPKDTLMISRTPEVTC |
| | A7 | VVVDVSHEDPEVKFNWYVDGVEVHNAKTKPREEQYNST |
| | | YRVVSVLTVLHQDWLNGKEYKCKVSNKALPAPIEKTISK |
| | | AKGQPREPQVYTLPPSREEMTKNQVSLTCLVKGFYPSDIA |
| | | VEWESNGQPENNYKTTPPVLDSDGSFFLYSKLTVDKSRW |
| | | QQGNVFSCSVMHEALHNHYTQKSLSLSPGKGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKC |
| | | EKPLPDQAFFVLHNMHSNCVSFECKTDPGVFIGVKDNHLA |
| | | LIKVDSSENLCTENILFKLSET |
| 139 | hIgG1 Fc-hIL-33 | DKTHTCPPCPAPELLGGPSVFLFPPKPKDTLMISRTPEVTC |
| | mutein (C208S, | VVVDVSHEDPEVKFNWYVDGVEVHNAKTKPREEQYNST |
| | C227S, C232S, | YRVVSVLTVLHQDWLNGKEYKCKVSNKALPAPIEKTISK |
| | C259S) | AKGQPREPQVYTLPPSREEMTKNQVSLTCLVKGFYPSDIA |
| | A8 | VEWESNGQPENNYKTTPPVLDSDGSFFLYSKLTVDKSRW |
| | | QQGNVFSCSVMHEALHNHYTQKSLSLSPGKGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKS |
| | | EKPLPDQAFFVLHNMHSNSVSFESKTDPGVFIGVKDNHLA |
| | | LIKVDSSENLSTENILFKLSET |
| 140 | hST2-hIgG1 Fc | KFSKQSWGLENEALIVRCPRQGKPSYTVDWYYSQTNKSIP |
| | A9 | TQERNRVFASGQLLKFLPAAVADSGIYTCIVRSPTFNRTGY |
| | | ANVTIYKKQSDCNVPDYLMYSTVSGSEKNSKIYCPTIDLY |
| | | NWTAPLEWFKNCQALQGSRYRAHKSFLVIDNVMTEDAG |
| | | DYTCKFIHNENGANYSVTATRSFTVKDEQGFSLFPVIGAPA |
| | | QNEIKEVEIGKNANLTCSACFGKGTQFLAAVLWQLNGTKI |
| | | TDFGEPRIQQEEGQNQSFSNGLACLDMVLRIADVKEEDLL |
| | | LQYDCLALNLHGLRRHTVRLSRKGGGGSDKTHTCPPCPA |
| | | PELLGGPSVFLFPPKPKDTLMISRTPEVTCVVVDVSHEDPE |
| | | VKFNWYVDGVEVHNAKTKPREEQYNSTYRVVSVLTVLH |
| | | QDWLNGKEYKCKVSNKALPAPIEKTISKAKGQPREPQVYT |
| | | LPPSREEMTKNQVSLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| 141 | hIgG1 (N297A, last | DKTHTCPPCPAPELLGGPSVFLFPPKPKDTLMISRTPEVTC |
| | K to A) Fc (knob)- | VVVDVSHEDPEVKFNWYVDGVEVHNAKTKPREEQYAST |
| | hST2-hIL-33 | YRVVSVLTVLHQDWLNGKEYKCKVSNKALPAPIEKTISK |
| | | AKGQPREPQVYTLPPCREEMTKNQVSLWCLVKGFYPSDIA |
| | | VEWESNGQPENNYKTTPPVLDSDGSFFLYSKLTVDKSRW |
| | | QQGNVFSCSVMHEALHNHYTQKSLSLSPGKGGGGSGGGG |
| | | SGGGGSKFSKQSWGLENEALIVRCPRQGKPSYTVDWYYS |
| | | QTNKSIPTQERNRVFASGQLLKFLPAAVADSGIYTCIVRSP |
| | | TFNRTGYANVTIYKKQSDCNVPDYLMYSTVSGSEKNSKIY |
| | | CPTIDLYNWTAPLEWFKNCQALQGSRYRAHKSFLVIDNV |
| | | MTEDAGDYTCKFIHNENGANYSVTATRSFTVKDEQGFSLF |
| | | PVIGAPAQNEIKEVEIGKNANLTCSACFGKGTQFLAAVLW |
| | | QLNGTKITDFGEPRIQQEEGQNQSFSNGLACLDMVLRIAD |
| | | VKEEDLLLQYDCLALNLHGLRRHTVRLSRKGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKC |
| | | EKPLPDQAFFVLHNMHSNCVSFECKTDPGVFIGVKDNHLA |
| | | LIKVDSSENLCTENILFKLSET |
| 142 | hIgG1 (N297A, last | DKTHTCPPCPAPELLGGPSVFLFPPKPKDTLMISRTPEVTC |
| | K to A) Fc (knob)- | VVVDVSHEDPEVKFNWYVDGVEVHNAKTKPREEQYAST |
| | hST2-hIL-33 mutein | YRVVSVLTVLHQDWLNGKEYKCKVSNKALPAPIEKTISK |
| | (C208S, C227S, | AKGQPREPQVYTLPPCREEMTKNQVSLWCLVKGFYPSDIA |
| | C232S, C259S) | VEWESNGQPENNYKTTPPVLDSDGSFFLYSKLTVDKSRW |
| | | QQGNVFSCSVMHEALHNHYTQKSLSLSPGKGGGGSGGGG |
| | | SGGGGSKFSKQSWGLENEALIVRCPRQGKPSYTVDWYYS |
| | | QTNKSIPTQERNRVFASGQLLKFLPAAVADSGIYTCIVRSP |
| | | TFNRTGYANVTIYKKQSDCNVPDYLMYSTVSGSEKNSKIY |
| | | CPTIDLYNWTAPLEWFKNCQALQGSRYRAHKSFLVIDNV |
| | | MTEDAGDYTCKFIHNENGANYSVTATRSFTVKDEQGFSLF |
| | | PVIGAPAQNEIKEVEIGKNANLTCSACFGKGTQFLAAVLW |
| | | QLNGTKITDFGEPRIQQEEGQNQSFSNGLACLDMVLRIAD |
| | | VKEEDLLLQYDCLALNLHGLRRHTVRLSRKGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKC |
| | | EKPLPDQAFFVLHNMHSNCVSFECKTDPGVFIGVKDNHLA |
| | | LIKVDSSENLCTENILFKLSET |
| 143 | hIgG1 (N297A, last | DKTHTCPPCPAPELLGGPSVFLFPPKPKDTLMISRTPEVTC |
| | K to A) Fc (hole) | VVVDVSHEDPEVKFNWYVDGVEVHNAKTKPREEQYAST |
| | | YRVVSVLTVLHQDWLNGKEYKCKVSNKALPAPIEKTISK |
| | | AKGQPREPQVCTLPPSREEMTKNQVSLSCAVKGFYPSDIA |
| | | VEWESNGQPENNYKTTPPVLDSDGSFFLVSKLTVDKSRW |
| | | QQGNVFSCSVMHEALHNHYTQKSLSLSPGA |
| 144 | hIgG1 (N297A, last | DKTHTCPPCPAPELLGGPSVFLFPPKPKDTLMISRTPEVTC |
| | K to A) Fc (hole)- | VVVDVSHEDPEVKFNWYVDGVEVHNAKTKPREEQYAST |
| | hST2-hIL-33 | YRVVSVLTVLHQDWLNGKEYKCKVSNKALPAPIEKTISK |
| | A12 | AKGQPREPQVCTLPPSREEMTKNQVSLSCAVKGFYPSDIA |
| | | VEWESNGQPENNYKTTPPVLDSDGSFFLVSKLTVDKSRW |
| | | QQGNVFSCSVMHEALHNHYTQKSLSLSPGAGGGGSGGGG |
| | | SGGGGSKFSKQSWGLENEALIVRCPRQGKPSYTVDWYYS |
| | | QTNKSIPTQERNRVFASGQLLKFLPAAVADSGIYTCIVRSP |
| | | TFNRTGYANVTIYKKQSDCNVPDYLMYSTVSGSEKNSKIY |
| | | CPTIDLYNWTAPLEWFKNCQALQGSRYRAHKSFLVIDNV |
| | | MTEDAGDYTCKFIHNENGANYSVTATRSFTVKDEQGFSLF |
| | | PVIGAPAQNEIKEVEIGKNANLTCSACFGKGTQFLAAVLW |
| | | QLNGTKITDFGEPRIQQEEGQNQSFSNGLACLDMVLRIAD |
| | | VKEEDLLLQYDCLALNLHGLRRHTVRLSRKGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKC |
| | | EKPLPDQAFFVLHNMHSNCVSFECKTDPGVFIGVKDNHLA |
| | | LIKVDSSENLCTENILFKLSET |
| 145 | hIgG1 (N297A, last | DKTHTCPPCPAPELLGGPSVFLFPPKPKDTLMISRTPEVTC |
| | K to A) Fc (hole) | VVVDVSHEDPEVKFNWYVDGVEVHNAKTKPREEQYAST |
| | A13 | YRVVSVLTVLHQDWLNGKEYKCKVSNKALPAPIEKTISK |
| | | AKGQPREPQVYTLPPCREEMTKNQVSLWCLVKGFYPSDIA |
| | | VEWESNGQPENNYKTTPPVLDSDGSFFLYSKLTVDKSRW |
| | | QQGNVFSCSVMHEALHNHYTQKSLSLSPGK |
| 146 | hIgG1 (N297A, last | DKTHTCPPCPAPELLGGPSVFLFPPKPKDTLMISRTPEVTC |
| | K to A) Fc (hole)- | VVVDVSHEDPEVKFNWYVDGVEVHNAKTKPREEQYAST |
| | hIL-33 | YRVVSVLTVLHQDWLNGKEYKCKVSNKALPAPIEKTISK |
| | | AKGQPREPQVCTLPPSREEMTKNQVSLSCAVKGFYPSDIA |
| | | VEWESNGQPENNYKTTPPVLDSDGSFFLVSKLTVDKSRW |
| | | QQGNVFSCSVMHEALHNHYTQKSLSLSPGAGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKC |
| | | EKPLPDQAFFVLHNMHSNCVSFECKTDPGVFIGVKDNHLA |
| | | LIKVDSSENLCTENILFKLSET |
| 147 | hIgG1 (N297A, last | DKTHTCPPCPAPELLGGPSVFLFPPKPKDTLMISRTPEVTC |
| | K to A) Fc (hole)- | VVVDVSHEDPEVKFNWYVDGVEVHNAKTKPREEQYAST |
| | hIL-33 mutein | YRVVSVLTVLHQDWLNGKEYKCKVSNKALPAPIEKTISK |
| | (C208S, C227S, | AKGQPREPQVCTLPPSREEMTKNQVSLSCAVKGFYPSDIA |
| | C232S, C259S) | VEWESNGQPENNYKTTPPVLDSDGSFFLVSKLTVDKSRW |
| | | QQGNVFSCSVMHEALHNHYTQKSLSLSPGAGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKS |
| | | EKPLPDQAFFVLHNMHSNSVSFESKTDPGVFIGVKDNHLA |
| | | LIKVDSSENLSTENILFKLSET |
| 148 | hIgG1 (N297A, last | DKTHTCPPCPAPELLGGPSVFLFPPKPKDTLMISRTPEVTC |
| | K to A) Fc (knob)- | VVVDVSHEDPEVKFNWYVDGVEVHNAKTKPREEQYAST |
| | hST2 | YRVVSVLTVLHQDWLNGKEYKCKVSNKALPAPIEKTISK |
| | | AKGQPREPQVYTLPPCREEMTKNQVSLWCLVKGFYPSDIA |
| | | VEWESNGQPENNYKTTPPVLDSDGSFFLYSKLTVDKSRW |
| | | QQGNVFSCSVMHEALHNHYTQKSLSLSPGKGGGGSGGGG |
| | | SGGGGSKFSKQSWGLENEALIVRCPRQGKPSYTVDWYYS |
| | | QTNKSIPTQERNRVFASGQLLKFLPAAVADSGIYTCIVRSP |
| | | TFNRTGYANVTIYKKQSDCNVPDYLMYSTVSGSEKNSKIY |
| | | CPTIDLYNWTAPLEWFKNCQALQGSRYRAHKSFLVIDNV |
| | | MTEDAGDYTCKFIHNENGANYSVTATRSFTVKDEQGFSLF |
| | | PVIGAPAQNEIKEVEIGKNANLTCSACFGKGTQFLAAVLW |
| | | QLNGTKITDFGEPRIQQEEGQNQSFSNGLACLDMVLRIAD |
| | | VKEEDLLLQYDCLALNLHGLRRHTVRLSRK |
| 149 | hIgG1 (N297A, last | DKTHTCPPCPAPELLGGPSVFLFPPKPKDTLMISRTPEVTC |
| | K to A) Fc-hST2- | VVVDVSHEDPEVKFNWYVDGVEVHNAKTKPREEQYAST |
| | hIL-33 | YRVVSVLTVLHQDWLNGKEYKCKVSNKALPAPIEKTISK |
| | A16 | AKGQPREPQVYTLPPSREEMTKNQVSLTCLVKGFYPSDIA |
| | | VEWESNGQPENNYKTTPPVLDSDGSFFLYSKLTVDKSRW |
| | | QQGNVFSCSVMHEALHNHYTQKSLSLSPGKGGGGSGGGG |
| | | SGGGGSKFSKQSWGLENEALIVRCPRQGKPSYTVDWYYS |
| | | QTNKSIPTQERNRVFASGQLLKFLPAAVADSGIYTCIVRSP |
| | | TFNRTGYANVTIYKKQSDCNVPDYLMYSTVSGSEKNSKIY |
| | | CPTIDLYNWTAPLEWFKNCQALQGSRYRAHKSFLVIDNV |
| | | MTEDAGDYTCKFIHNENGANYSVTATRSFTVKDEQGFSLF |
| | | PVIGAPAQNEIKEVEIGKNANLTCSACFGKGTQFLAAVLW |
| | | QLNGTKITDFGEPRIQQEEGQNQSFSNGLACLDMVLRIAD |
| | | VKEEDLLLQYDCLALNLHGLRRHTVRLSRKGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKC |
| | | EKPLPDQAFFVLHNMHSNCVSFECKTDPGVFIGVKDNHLA |
| | | LIKVDSSENLCTENILFKLSET |
| 150 | hIgG1 (N297A, last | DKTHTCPPCPAPELLGGPSVFLFPPKPKDTLMISRTPEVTC |
| | K to A) Fc-hST2- | VVVDVSHEDPEVKFNWYVDGVEVHNAKTKPREEQYAST |
| | hIL-33 | YRVVSVLTVLHQDWLNGKEYKCKVSNKALPAPIEKTISK |
| | A17 | AKGQPREPQVYTLPPSREEMTKNQVSLTCLVKGFYPSDIA |
| | | VEWESNGQPENNYKTTPPVLDSDGSFFLYSKLTVDKSRW |
| | | QQGNVFSCSVMHEALHNHYTQKSLSLSPGKGGGGSGGGG |
| | | SGGGGSKFSKQSWGLENEALIVRCPRQGKPSYTVDWYYS |
| | | QTNKSIPTQERNRVFASGQLLKFLPAAVADSGIYTCIVRSP |
| | | TFNRTGYANVTIYKKQSDCNVPDYLMYSTVSGSEKNSKIY |
| | | CPTIDLYNWTAPLEWFKNCQALQGSRYRAHKSFLVIDNV |
| | | MTEDAGDYTCKFIHNENGANYSVTATRSFTVKDEQGFSLF |
| | | PVIGAPAQNEIKEVEIGKNANLTCSACFGKGTQFLAAVLW |
| | | QLNGTKITDFGEPRIQQEEGQNQSFSNGLACLDMVLRIAD |
| | | VKEEDLLLQYDCLALNLHGLRRHTVRLSRKGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKS |
| | | EKPLPDQAFFVLHNMHSNSVSFESKTDPGVFIGVKDNHLA |
| | | LIKVDSSENLSTENILFKLSET |
| 151 | Anti-PD-L1-HC | EVQLVESGGGLVQPGGSLRLSCAASGFTFSDSWIHWVRQ |
| | (last K to A) | APGKGLEWVAWISPYGGSTYYADSVKGRFTISADTSKNT |
| | (hole)-hIL-33 | AYLQMNSLRAEDTAVYYCARRHWPGGFDYWGQGTLVT |
| | | VSSASTKGPSVFPLAPSSKSTSGGTAALGCLVKDYFPEPVT |
| | | VSWNSGALTSGVHTFPAVLQSSGLYSLSSVVTVPSSSLGT |
| | | QTYICNVNHKPSNTKVDKKVEPKSCDKTHTCPPCPAPELL |
| | | GGPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVCTLPPSR |
| | | EEMTKNQVSLSCAVKGFYPSDIAVEWESNGQPENNYKTT |
| | | PPVLDSDGSFFLVSKLTVDKSRWQQGNVFSCSVMHEALH |
| | | NHYTQKSLSLSPGAGGGGSGGGGSGGGGSAFGISGVQKY |
| | | TRALHDSSITGISPITEYLASLSTYNDQSITFALEDESYEIYV |
| | | EDLKKDEKKDKVLLSYYESQHPSNESGDGVDGKMLMVT |
| | | LSPTKDFWLHANNKEHSVELHKCEKPLPDQAFFVLHNMH |
| | | SNCVSFECKTDPGVFIGVKDNHLALIKVDSSENLCTENILF |
| | | KLSET |
| 152 | Anti-PD-L1-HC | EVQLVESGGGLVQPGGSLRLSCAASGFTFSDSWIHWVRQ |
| | (last K to A) | APGKGLEWVAWISPYGGSTYYADSVKGRFTISADTSKNT |
| | (hole)- | AYLQMNSLRAEDTAVYYCARRHWPGGFDYWGQGTLVT |
| | hIL-33 mutein | VSSASTKGPSVFPLAPSSKSTSGGTAALGCLVKDYFPEPVT |
| | (C208S, C227S, | VSWNSGALTSGVHTFPAVLQSSGLYSLSSVVTVPSSSLGT |
| | C232S, C259S) | QTYICNVNHKPSNTKVDKKVEPKSCDKTHTCPPCPAPELL |
| | | GGPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVCTLPPSR |
| | | EEMTKNQVSLSCAVKGFYPSDIAVEWESNGQPENNYKTT |
| | | PPVLDSDGSFFLVSKLTVDKSRWQQGNVFSCSVMHEALH |
| | | NHYTQKSLSLSPGAGGGGSGGGGSGGGGSAFGISGVQKY |
| | | TRALHDSSITGISPITEYLASLSTYNDQSITFALEDESYEIYV |
| | | EDLKKDEKKDKVLLSYYESQHPSNESGDGVDGKMLMVT |
| | | LSPTKDFWLHANNKEHSVELHKSEKPLPDQAFFVLHNMH |
| | | SNSVSFESKTDPGVFIGVKDNHLALIKVDSSENLSTENILFK |
| | | LSET |
| 153 | Anti-PD-L1-HC | EVQLVESGGGLVQPGGSLRLSCAASGFTFSDSWIHWVRQ |
| | (last K to A) | APGKGLEWVAWISPYGGSTYYADSVKGRFTISADTSKNT |
| | (knob)-hST2 | AYLQMNSLRAEDTAVYYCARRHWPGGFDYWGQGTLVT |
| | | VSSASTKGPSVFPLAPSSKSTSGGTAALGCLVKDYFPEPVT |
| | | VSWNSGALTSGVHTFPAVLQSSGLYSLSSVVTVPSSSLGT |
| | | QTYICNVNHKPSNTKVDKKVEPKSCDKTHTCPPCPAPELL |
| | | GGPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPC |
| | | REEMTKNQVSLWCLVKGFYPSDIAVEWESNGQPENNYKT |
| | | TPPVLDSDGSFFLYSKLTVDKSRWQQGNVFSCSVMHEAL |
| | | HNHYTQKSLSLSPGAGGGGSGGGGSGGGGSKFSKQSWGL |
| | | ENEALIVRCPRQGKPSYTVDWYYSQTNKSIPTQERNRVFA |
| | | SGQLLKFLPAAVADSGIYTCIVRSPTFNRTGYANVTIYKKQ |
| | | SDCNVPDYLMYSTVSGSEKNSKIYCPTIDLYNWTAPLEWF |
| | | KNCQALQGSRYRAHKSFLVIDNVMTEDAGDYTCKFIHNE |
| | | NGANYSVTATRSFTVKDEQGFSLFPVIGAPAQNEIKEVEIG |
| | | KNANLTCSACFGKGTQFLAAVLWQLNGTKITDFGEPRIQQ |
| | | EEGQNQSFSNGLACLDMVLRIADVKEEDLLLQYDCLALN |
| | | LHGLRRHTVRLSRK |
| 154 | Anti-PD-L1-HC | EVQLVESGGGLVQPGGSLRLSCAASGFTFSDSWIHWVRQ |
| | (last K to A) | APGKGLEWVAWISPYGGSTYYADSVKGRFTISADTSKNT |
| | (hole)-hST2-hIL-33 | AYLQMNSLRAEDTAVYYCARRHWPGGFDYWGQGTLVT |
| | | VSSASTKGPSVFPLAPSSKSTSGGTAALGCLVKDYFPEPVT |
| | | VSWNSGALTSGVHTFPAVLQSSGLYSLSSVVTVPSSSLGT |
| | | QTYICNVNHKPSNTKVDKKVEPKSCDKTHTCPPCPAPELL |
| | | GGPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVCTLPPSR |
| | | EEMTKNQVSLSCAVKGFYPSDIAVEWESNGQPENNYKTT |
| | | PPVLDSDGSFFLVSKLTVDKSRWQQGNVFSCSVMHEALH |
| | | NHYTQKSLSLSPGAGGGGSGGGGSGGGGSKFSKQSWGLE |
| | | NEALIVRCPRQGKPSYTVDWYYSQTNKSIPTQERNRVFAS |
| | | GQLLKFLPAAVADSGIYTCIVRSPTFNRTGYANVTIYKKQS |
| | | DCNVPDYLMYSTVSGSEKNSKIYCPTIDLYNWTAPLEWFK |
| | | NCQALQGSRYRAHKSFLVIDNVMTEDAGDYTCKFIHNEN |
| | | GANYSVTATRSFTVKDEQGFSLFPVIGAPAQNEIKEVEIGK |
| | | NANLTCSACFGKGTQFLAAVLWQLNGTKITDFGEPRIQQE |
| | | EGQNQSFSNGLACLDMVLRIADVKEEDLLLQYDCLALNL |
| | | HGLRRHTVRLSRKGGGSGGGSGGGSGGGSAFGISGVQKY |
| | | TRALHDSSITGISPITEYLASLSTYNDQSITFALEDESYEIYV |
| | | EDLKKDEKKDKVLLSYYESQHPSNESGDGVDGKMLMVT |
| | | LSPTKDFWLHANNKEHSVELHKCEKPLPDQAFFVLHNMH |
| | | SNCVSFECKTDPGVFIGVKDNHLALIKVDSSENLCTENILF |
| | | KLSET |
| 155 | Anti-PD-L1-HC | EVQLVESGGGLVQPGGSLRLSCAASGFTFSDSWIHWVRQ |
| | (last K to A) | APGKGLEWVAWISPYGGSTYYADSVKGRFTISADTSKNT |
| | (hole)- | AYLQMNSLRAEDTAVYYCARRHWPGGFDYWGQGTLVT |
| | hST2-hIL-33 mutein | VSSASTKGPSVFPLAPSSKSTSGGTAALGCLVKDYFPEPVT |
| | (C208S, C227S, | VSWNSGALTSGVHTFPAVLQSSGLYSLSSVVTVPSSSLGT |
| | C232S, C259S) | QTYICNVNHKPSNTKVDKKVEPKSCDKTHTCPPCPAPELL |
| | | GGPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVCTLPPSR |
| | | EEMTKNQVSLSCAVKGFYPSDIAVEWESNGQPENNYKTT |
| | | PPVLDSDGSFFLVSKLTVDKSRWQQGNVFSCSVMHEALH |
| | | NHYTQKSLSLSPGAGGGGSGGGGSGGGGSKFSKQSWGLE |
| | | NEALIVRCPRQGKPSYTVDWYYSQTNKSIPTQERNRVFAS |
| | | GQLLKFLPAAVADSGIYTCIVRSPTFNRTGYANVTIYKKQS |
| | | DCNVPDYLMYSTVSGSEKNSKIYCPTIDLYNWTAPLEWFK |
| | | NCQALQGSRYRAHKSFLVIDNVMTEDAGDYTCKFIHNEN |
| | | GANYSVTATRSFTVKDEQGFSLFPVIGAPAQNEIKEVEIGK |
| | | NANLTCSACFGKGTQFLAAVLWQLNGTKITDFGEPRIQQE |
| | | EGQNQSFSNGLACLDMVLRIADVKEEDLLLQYDCLALNL |
| | | HGLRRHTVRLSRKGGGSGGGSGGGSGGGSAFGISGVQKY |
| | | TRALHDSSITGISPITEYLASLSTYNDQSITFALEDESYEIYV |
| | | EDLKKDEKKDKVLLSYYESQHPSNESGDGVDGKMLMVT |
| | | LSPTKDFWLHANNKEHSVELHKSEKPLPDQAFFVLHNMH |
| | | SNSVSFESKTDPGVFIGVKDNHLALIKVDSSENLSTENILFK |
| | | LSET |
| 156 | Anti-PD-L1-HC | EVQLVESGGGLVQPGGSLRLSCAASGFTFSDSWIHWVRQ |
| | (last K to A) | APGKGLEWVAWISPYGGSTYYADSVKGRFTISADTSKNT |
| | (knob) | AYLQMNSLRAEDTAVYYCARRHWPGGFDYWGQGTLVT |
| | | VSSASTKGPSVFPLAPSSKSTSGGTAALGCLVKDYFPEPVT |
| | | VSWNSGALTSGVHTFPAVLQSSGLYSLSSVVTVPSSSLGT |
| | | QTYICNVNHKPSNTKVDKKVEPKSCDKTHTCPPCPAPELL |
| | | GGPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPC |
| | | REEMTKNQVSLWCLVKGFYPSDIAVEWESNGQPENNYKT |
| | | TPPVLDSDGSFFLYSKLTVDKSRWQQGNVFSCSVMHEAL |
| | | HNHYTQKSLSLSPGA |
| 157 | Anti-PD-L1-HC | EVQLVESGGGLVQPGGSLRLSCAASGFTFSDSWIHWVRQ |
| | (last K to A)-hST2- | APGKGLEWVAWISPYGGSTYYADSVKGRFTISADTSKNT |
| | hIL-33 | AYLQMNSLRAEDTAVYYCARRHWPGGFDYWGQGTLVT |
| | | VSSASTKGPSVFPLAPSSKSTSGGTAALGCLVKDYFPEPVT |
| | | VSWNSGALTSGVHTFPAVLQSSGLYSLSSVVTVPSSSLGT |
| | | QTYICNVNHKPSNTKVDKKVEPKSCDKTHTCPPCPAPELL |
| | | GGPSVFLFPPKPKDTLMISRTPEVTCVVVDVSHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EEMTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPGAGGGGSGGGGSGGGGSKFSKQSWGLEN |
| | | EALIVRCPRQGKPSYTVDWYYSQTNKSIPTQERNRVFASG |
| | | QLLKFLPAAVADSGIYTCIVRSPTFNRTGYANVTIYKKQSD |
| | | CNVPDYLMYSTVSGSEKNSKIYCPTIDLYNWTAPLEWFKN |
| | | CQALQGSRYRAHKSFLVIDNVMTEDAGDYTCKFIHNENG |
| | | ANYSVTATRSFTVKDEQGFSLFPVIGAPAQNEIKEVEIGKN |
| | | ANLTCSACFGKGTQFLAAVLWQLNGTKITDFGEPRIQQEE |
| | | GQNQSFSNGLACLDMVLRIADVKEEDLLLQYDCLALNLH |
| | | GLRRHTVRLSRKGGGSGGGSGGGSGGGSAFGISGVQKYT |
| | | RALHDSSITGISPITEYLASLSTYNDQSITFALEDESYEIYVE |
| | | DLKKDEKKDKVLLSYYESQHPSNESGDGVDGKMLMVTL |
| | | SPTKDFWLHANNKEHSVELHKCEKPLPDQAFFVLHNMHS |
| | | NCVSFECKTDPGVFIGVKDNHLALIKVDSSENLCTENILFK |
| | | LSET |
| 158 | Anti-PD-L1-HC | EVQLVESGGGLVQPGGSLRLSCAASGFTFSDSWIHWVRQ |
| | (last K to A)-hST2- | APGKGLEWVAWISPYGGSTYYADSVKGRFTISADTSKNT |
| | hIL-33 mutein | AYLQMNSLRAEDTAVYYCARRHWPGGFDYWGQGTLVT |
| | (C208S, C227S, | VSSASTKGPSVFPLAPSSKSTSGGTAALGCLVKDYFPEPVT |
| | C232S, C259S) | VSWNSGALTSGVHTFPAVLQSSGLYSLSSVVTVPSSSLGT |
| | | QTYICNVNHKPSNTKVDKKVEPKSCDKTHTCPPCPAPELL |
| | | GGPSVFLFPPKPKDTLMISRTPEVTCVVVDVSHEDPEVKEN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EEMTKNQVSLTCLVKGFYPSDIAVEWESNGQPENNYKTTP |
| | | PVLDSDGSFFLYSKLTVDKSRWQQGNVFSCSVMHEALHN |
| | | HYTQKSLSLSPGAGGGGSGGGGSGGGGSKFSKQSWGLEN |
| | | EALIVRCPRQGKPSYTVDWYYSQTNKSIPTQERNRVFASG |
| | | QLLKFLPAAVADSGIYTCIVRSPTFNRTGYANVTIYKKQSD |
| | | CNVPDYLMYSTVSGSEKNSKIYCPTIDLYNWTAPLEWFKN |
| | | CQALQGSRYRAHKSFLVIDNVMTEDAGDYTCKFIHNENG |
| | | ANYSVTATRSFTVKDEQGFSLFPVIGAPAQNEIKEVEIGKN |
| | | ANLTCSACFGKGTQFLAAVLWQLNGTKITDFGEPRIQQEE |
| | | GQNQSFSNGLACLDMVLRIADVKEEDLLLQYDCLALNLH |
| | | GLRRHTVRLSRKGGGSGGGSGGGSGGGSAFGISGVQKYT |
| | | RALHDSSITGISPITEYLASLSTYNDQSITFALEDESYEIYVE |
| | | DLKKDEKKDKVLLSYYESQHPSNESGDGVDGKMLMVTL |
| | | SPTKDFWLHANNKEHSVELHKSEKPLPDQAFFVLHNMHS |
| | | NSVSFESKTDPGVFIGVKDNHLALIKVDSSENLSTENILFKL |
| | | SET |
| 159 | Anti-PD-L1-LC | DIQMTQSPSSLSASVGDRVTITCRASQDVSTAVAWYQQKP |
| | (kappa) | GKAPKLLIYSASFLYSGVPSRFSGSGSGTDFTLTISSLQPED |
| | | FATYYCQQYLYHPATFGQGTKVEIKRTVAAPSVFIFPPSDE |
| | | QLKSGTASVVCLLNNFYPREAKVQWKVDNALQSGNSQES |
| | | VTEQDSKDSTYSLSSTLTLSKADYEKHKVYACEVTHQGLS |
| | | SPVTKSFNRGEC |
| 160 | Anti-MSLN-hIgG1 | QVQLVESGGGLVQPGGSLRLSCAASGITFPVNAYGWYRQ |
| | (last K to A)-hIL-33 | APGKQRDLVAIISAGGTTNYADSVKGRFTISRDNSKNTLY |
| | (knob) | LQMNSLRAEDTAVYYCYLQRRIGMLRDYWGQGTQVTVS |
| | | SDKTHTCPPCPAPELLGGPSVFLFPPKPKDTLMISRTPEVTC |
| | | VVVDVSHEDPEVKFNWYVDGVEVHNAKTKPREEQYNST |
| | | YRVVSVLTVLHQDWLNGKEYKCKVSNKALPAPIEKTISK |
| | | AKGQPREPQVYTLPPCREEMTKNQVSLWCLVKGFYPSDIA |
| | | VEWESNGQPENNYKTTPPVLDSDGSFFLYSKLTVDKSRW |
| | | QQGNVFSCSVMHEALHNHYTQKSLSLSPGAGGGGSGGGG |
| | | SGGGGSAFGISGVQKYTRALHDSSITGISPITEYLASLSTYN |
| | | DQSITFALEDESYEIYVEDLKKDEKKDKVLLSYYESQHPSN |
| | | ESGDGVDGKMLMVTLSPTKDFWLHANNKEHSVELHKCE |
| | | KPLPDQAFFVLHNMHSNCVSFECKTDPGVFIGVKDNHLAL |
| | | IKVDSSENLCTENILFKLSET |
| 161 | Anti-MSLN-hIgG1 | QVQLVESGGGLVQPGGSLRLSCAASGITFPVNAYGWYRQ |
| | (last K to A)-hIL-33 | APGKQRDLVAIISAGGTTNYADSVKGRFTISRDNSKNTLY |
| | mutein (knob) | LQMNSLRAEDTAVYYCYLQRRIGMLRDYWGQGTQVTVS |
| | (C208S, C227S, | SDKTHTCPPCPAPELLGGPSVFLFPPKPKDTLMISRTPEVTC |
| | C232S, C259S) | VVVDVSHEDPEVKFNWYVDGVEVHNAKTKPREEQYNST |
| | | YRVVSVLTVLHQDWLNGKEYKCKVSNKALPAPIEKTISK |
| | | AKGQPREPQVYTLPPCREEMTKNQVSLWCLVKGFYPSDIA |
| | | VEWESNGQPENNYKTTPPVLDSDGSFFLYSKLTVDKSRW |
| | | QQGNVFSCSVMHEALHNHYTQKSLSLSPGAGGGGSGGGG |
| | | SGGGGSAFGISGVQKYTRALHDSSITGISPITEYLASLSTYN |
| | | DQSITFALEDESYEIYVEDLKKDEKKDKVLLSYYESQHPSN |
| | | ESGDGVDGKMLMVTLSPTKDFWLHANNKEHSVELHKSE |
| | | KPLPDQAFFVLHNMHSNSVSFESKTDPGVFIGVKDNHLAL |
| | | IKVDSSENLSTENILFKLSET |
| 162 | Anti-MSLN-hIgG1 | QVQLVESGGGLVQPGGSLRLSCAASGITFPVNAYGWYRQ |
| | (last K to A)-hST2 | APGKQRDLVAIISAGGTTNYADSVKGRFTISRDNSKNTLY |
| | (hole) | LQMNSLRAEDTAVYYCYLQRRIGMLRDYWGQGTQVTVS |
| | | SDKTHTCPPCPAPELLGGPSVFLFPPKPKDTLMISRTPEVTC |
| | | VVVDVSHEDPEVKFNWYVDGVEVHNAKTKPREEQYNST |
| | | YRVVSVLTVLHQDWLNGKEYKCKVSNKALPAPIEKTISK |
| | | AKGQPREPQVCTLPPSREEMTKNQVSLSCAVKGFYPSDIA |
| | | VEWESNGQPENNYKTTPPVLDSDGSFFLVSKLTVDKSRW |
| | | QQGNVFSCSVMHEALHNHYTQKSLSLSPGAGGGGGGGG |
| | | SGGGGSKFSKQSWGLENEALIVRCPRQGKPSYTVDWYYS |
| | | QTNKSIPTQERNRVFASGQLLKFLPAAVADSGIYTCIVRSP |
| | | TFNRTGYANVTIYKKQSDCNVPDYLMYSTVSGSEKNSKIY |
| | | CPTIDLYNWTAPLEWFKNCQALQGSRYRAHKSFLVIDNV |
| | | MTEDAGDYTCKFIHNENGANYSVTATRSFTVKDEQGFSLF |
| | | PVIGAPAQNEIKEVEIGKNANLTCSACFGKGTQFLAAVLW |
| | | QLNGTKITDFGEPRIQQEEGQNQSFSNGLACLDMVLRIAD |
| | | VKEEDLLLQYDCLALNLHGLRRHTVRLSRK |
| 163 | Anti-MSLN-hIgG1 | QVQLVESGGGLVQPGGSLRLSCAASGITFPVNAYGWYRQ |
| | (last K to A)-hST2- | APGKQRDLVAIISAGGTTNYADSVKGRFTISRDNSKNTLY |
| | hIL-33 (hole) | LQMNSLRAEDTAVYYCYLQRRIGMLRDYWGQGTQVTVS |
| | | SDKTHTCPPCPAPELLGGPSVFLFPPKPKDTLMISRTPEVTC |
| | | VVVDVSHEDPEVKFNWYVDGVEVHNAKTKPREEQYNST |
| | | YRVVSVLTVLHQDWLNGKEYKCKVSNKALPAPIEKTISK |
| | | AKGQPREPQVCTLPPSREEMTKNQVSLSCAVKGFYPSDIA |
| | | VEWESNGQPENNYKTTPPVLDSDGSFFLVSKLTVDKSRW |
| | | QQGNVFSCSVMHEALHNHYTQKSLSLSPGAGGGGSGGGG |
| | | SGGGGSKFSKQSWGLENEALIVRCPRQGKPSYTVDWYYS |
| | | QTNKSIPTQERNRVFASGQLLKFLPAAVADSGIYTCIVRSP |
| | | TFNRTGYANVTIYKKQSDCNVPDYLMYSTVSGSEKNSKIY |
| | | CPTIDLYNWTAPLEWFKNCQALQGSRYRAHKSFLVIDNV |
| | | MTEDAGDYTCKFIHNENGANYSVTATRSFTVKDEQGFSLF |
| | | PVIGAPAQNEIKEVEIGKNANLTCSACFGKGTQFLAAVLW |
| | | QLNGTKITDFGEPRIQQEEGQNQSFSNGLACLDMVLRIAD |
| | | VKEEDLLLQYDCLALNLHGLRRHTVRLSRKGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKC |
| | | EKPLPDQAFFVLHNMHSNCVSFECKTDPGVFIGVKDNHLA |
| | | LIKVDSSENLCTENILFKLSET |
| 164 | Anti-MSLN-hIgG1 | QVQLVESGGGLVQPGGSLRLSCAASGITFPVNAYGWYRQ |
| | (knob) | APGKQRDLVAIISAGGTTNYADSVKGRFTISRDNSKNTLY |
| | | LQMNSLRAEDTAVYYCYLQRRIGMLRDYWGQGTQVTVS |
| | | SDKTHTCPPCPAPELLGGPSVFLFPPKPKDTLMISRTPEVTC |
| | | VVVDVSHEDPEVKFNWYVDGVEVHNAKTKPREEQYNST |
| | | YRVVSVLTVLHQDWLNGKEYKCKVSNKALPAPIEKTISK |
| | | AKGQPREPQVYTLPPCREEMTKNQVSLWCLVKGFYPSDIA |
| | | VEWESNGQPENNYKTTPPVLDSDGSFFLYSKLTVDKSRW |
| | | QQGNVFSCSVMHEALHNHYTQKSLSLSPGK |
| 165 | Anti-MSLN-hIgG1 | QVQLVESGGGLVQPGGSLRLSCAASGITFPVNAYGWYRQ |
| | (last K to A)-hST2- | APGKQRDLVAIISAGGTTNYADSVKGRFTISRDNSKNTLY |
| | hIL-33 mutein | LQMNSLRAEDTAVYYCYLQRRIGMLRDYWGQGTQVTVS |
| | (knob) | SDKTHTCPPCPAPELLGGPSVFLFPPKPKDTLMISRTPEVTC |
| | (C208S, C227S, | VVVDVSHEDPEVKFNWYVDGVEVHNAKTKPREEQYNST |
| | C232S, C259S) | YRVVSVLTVLHQDWLNGKEYKCKVSNKALPAPIEKTISK |
| | | AKGQPREPQVYTLPPCREEMTKNQVSLWCLVKGFYPSDIA |
| | | VEWESNGQPENNYKTTPPVLDSDGSFFLYSKLTVDKSRW |
| | | QQGNVFSCSVMHEALHNHYTQKSLSLSPGAGGGGSGGGG |
| | | SGGGGSKFSKQSWGLENEALIVRCPRQGKPSYTVDWYYS |
| | | QTNKSIPTQERNRVFASGQLLKFLPAAVADSGIYTCIVRSP |
| | | TFNRTGYANVTIYKKQSDCNVPDYLMYSTVSGSEKNSKIY |
| | | CPTIDLYNWTAPLEWFKNCQALQGSRYRAHKSFLVIDNV |
| | | MTEDAGDYTCKFIHNENGANYSVTATRSFTVKDEQGFSLF |
| | | PVIGAPAQNEIKEVEIGKNANLTCSACFGKGTQFLAAVLW |
| | | QLNGTKITDFGEPRIQQEEGQNQSFSNGLACLDMVLRIAD |
| | | VKEEDLLLQYDCLALNLHGLRRHTVRLSRKGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKS |
| | | EKPLPDQAFFVLHNMHSNSVSFESKTDPGVFIGVKDNHLA |
| | | LIKVDSSENLSTENILFKLSET |
| 166 | Anti-MSLN-hIgG1 | QVQLVESGGGLVQPGGSLRLSCAASGITFPVNAYGWYRQ |
| | (last K to A) (hole) | APGKQRDLVAIISAGGTTNYADSVKGRFTISRDNSKNTLY |
| | | LQMNSLRAEDTAVYYCYLQRRIGMLRDYWGQGTQVTVS |
| | | SDKTHTCPPCPAPELLGGPSVFLFPPKPKDTLMISRTPEVTC |
| | | VVVDVSHEDPEVKFNWYVDGVEVHNAKTKPREEQYNST |
| | | YRVVSVLTVLHQDWLNGKEYKCKVSNKALPAPIEKTISK |
| | | AKGQPREPQVCTLPPSREEMTKNQVSLSCAVKGFYPSDIA |
| | | VEWESNGQPENNYKTTPPVLDSDGSFFLVSKLTVDKSRW |
| | | QQGNVFSCSVMHEALHNHYTQKSLSLSPGA |
| 167 | Anti-MSLN-hIgG1- | QVQLVESGGGLVQPGGSLRLSCAASGITFPVNAYGWYRQ |
| | hST2-hIL-33 | APGKQRDLVAIISAGGTTNYADSVKGRFTISRDNSKNTLY |
| | A28 | LQMNSLRAEDTAVYYCYLQRRIGMLRDYWGQGTQVTVS |
| | | SDKTHTCPPCPAPELLGGPSVFLFPPKPKDTLMISRTPEVTC |
| | | VVVDVSHEDPEVKFNWYVDGVEVHNAKTKPREEQYNST |
| | | YRVVSVLTVLHQDWLNGKEYKCKVSNKALPAPIEKTISK |
| | | AKGQPREPQVYTLPPSREEMTKNQVSLTCLVKGFYPSDIA |
| | | VEWESNGQPENNYKTTPPVLDSDGSFFLYSKLTVDKSRW |
| | | QQGNVFSCSVMHEALHNHYTQKSLSLSPGAGGGGSGGGG |
| | | SGGGGSKFSKQSWGLENEALIVRCPRQGKPSYTVDWYYS |
| | | QTNKSIPTQERNRVFASGQLLKFLPAAVADSGIYTCIVRSP |
| | | TFNRTGYANVTIYKKQSDCNVPDYLMYSTVSGSEKNSKIY |
| | | CPTIDLYNWTAPLEWFKNCQALQGSRYRAHKSFLVIDNV |
| | | MTEDAGDYTCKFIHNENGANYSVTATRSFTVKDEQGFSLF |
| | | PVIGAPAQNEIKEVEIGKNANLTCSACFGKGTQFLAAVLW |
| | | QLNGTKITDFGEPRIQQEEGQNQSFSNGLACLDMVLRIAD |
| | | VKEEDLLLQYDCLALNLHGLRRHTVRLSRKGGGSGGGSG |
| | | GGSGGGSAFGISGVQKYTRALHDSSITGISPITEYLASLSTY |
| | | NDQSITFALEDESYEIYVEDLKKDEKKDKVLLSYYESQHPS |
| | | NESGDGVDGKMLMVTLSPTKDFWLHANNKEHSVELHKC |
| | | EKPLPDQAFFVLHNMHSNCVSFECKTDPGVFIGVKDNHLA |
| | | LIKVDSSENLCTENILFKLSET |

The examples set forth above are provided to give those of ordinary skill in the art with a complete disclosure and description of how to make and use the claimed embodiments and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A fusion protein comprising a suppression of tumorigenicity 2 (ST2) domain or interleukin-33 (IL-33) domain, and a half-life-extension domain.

2. The fusion protein of claim 1, wherein the half-life-extension domain comprises an albumin binding domain, a fragment crystallizable (Fc) domain, a serum albumin, a polyethylene glycol group, or a fatty acyl group.

3. The fusion protein of claim 1 or 2, wherein the half-life-extension domain is an albumin binding domain.

4. The fusion protein of claim 2 or 3, wherein the albumin binding domain is an antibody or a fragment thereof that binds to a human serum albumin (HSA).

5. The fusion protein of claim 4, wherein the albumin binding domain is an anti-HSA single domain antibody.

6. The fusion protein of claim 4 or 5, wherein the albumin binding domain is an anti-HSA V_{H}H single domain antibody.

7. The fusion protein of any one of claims 1 to 6, comprising an IL-33 domain, an anti-HSA V_{H}H single domain antibody, and optionally a peptide linker; wherein the N-terminus of the IL-33 domain is connected to the C-terminus of the anti-HSA V_{H}H single domain antibody directly or via the peptide linker.

8. The fusion protein of any one of claims 1 to 6, comprising an ST2 domain, an anti-HSA V_{H}H single domain antibody, and a peptide linker; wherein the C-terminus of the ST2 domain is connected to the N-terminus of the anti-HSA V_{H}H single domain antibody directly or via the peptide linker.

9. The fusion protein of any one of claims 1 to 8, comprising an IL-33 domain, an ST2 domain, and an anti-HSA single domain antibody.

10. The fusion protein of any one of claims 1 to 9, comprising an IL-33 domain, an ST2 domain, an anti-HSA V_{H}H single domain antibody, and optionally first and second peptide linkers; wherein the C-terminus of the ST2 domain is connected to the N-terminus of the anti-HSA V_{H}H single domain antibody directly or via the first peptide linker, and the C-terminus of the anti-HSA V_{H}H single domain antibody is connected to the N-terminus of the IL-33 domain directly or via the second peptide linker.

11. The fusion protein of claim 10, wherein the first peptide linker is cleavable and the second peptide linker is non-cleavable.

12. The fusion protein of claim 10, wherein the first peptide linker is non-cleavable and the second peptide linker is cleavable.

13. The fusion protein of any one of claims 5 to 12, wherein the single domain antibody comprises (i) a CDR1 of SEQ ID NO: 1, a CDR2 of SEQ ID NO: 2, and a CDR3 of SEQ ID NO: 3; or (ii) a CDR1 of SEQ ID NO: 9, a CDR2 of SEQ ID NO: 10, and a CDR3 of SEQ ID NO: 11.

14. The fusion protein of any one of claims 5 to 13, wherein the single domain antibody comprises a CDR1 of SEQ ID NO: 1, a CDR2 of SEQ ID NO: 2, and a CDR3 of SEQ ID NO: 3.

15. The fusion protein of any one of claims 5 to 14, wherein the single domain antibody has an amino acid sequence of SEQ ID NO: 8.

16. The fusion protein of any one of claims 5 to 13, wherein the single domain antibody comprises a CDR1 of SEQ ID NO: 9, a CDR2 of SEQ ID NO: 10, and a CDR3 of SEQ ID NO: 11.

17. The fusion protein of any one of claims 5 to 13 and 16, wherein the single domain antibody has an amino acid sequence of SEQ ID NO: 15.

18. The fusion protein of claim 1 or 2, wherein the half-life-extension domain is a Fc domain.

19. The fusion protein of claim 1 or 18, comprising first and second IL-33 domains, an Fc domain comprising first and second peptide chains, and optionally first and second peptide linkers; wherein the N-terminus of the first IL-33 domain is connected to the C-terminus of the first peptide chain of the Fc domain directly or via the first peptide linker, and the N-terminus of the second IL-33 domain is connected to the C-terminus of the second peptide chain of the Fc domain directly or via the second peptide linker.

20. The fusion protein of claim 1 or 18, comprising first and second ST2 domains, an Fc domain comprising first and second peptide chains, and optionally first and second peptide linkers; wherein the N-terminus of the first ST2 domain is connected to the C-terminus of the first peptide chain of the Fc domain directly or via the first peptide linker, and the N-terminus of the second ST2 domain is connected to the C-terminus of the second peptide chain of the Fc domain directly or via the second peptide linker.

21. The fusion protein of claim 1 or 18, comprising first and second ST2 domains, an Fc domain comprising first and second peptide chains, and optionally first and second peptide linkers; wherein the C-terminus of the first ST2 domain is connected to the N-terminus of the first peptide chain of the Fc domain directly or via the first peptide linker, and the C-terminus of the second ST2 domain is connected to the N-terminus of the second peptide chain of the Fc domain directly or via the second peptide linker.

22. The fusion protein of claim 1 or 18, comprising an IL-33 domain, an ST2 domain, and an Fc domain.

23. The fusion protein of claim 1 or 22, comprising an IL-33 domain, an ST2 domain, an Fc domain comprising first and second peptide chains, and optionally first and second peptide linkers; wherein the N-terminus of the IL-33 domain is connected to the C-terminus of the ST2 domain directly or via the first peptide linker, and the N-terminus of the ST2 domain is connected to the C-terminus of the first peptide chain of the Fc domain directly or via the second peptide linker.

24. The fusion protein of claim 1 or 22, comprising an IL-33 domain, an ST2 domain, an Fc domain comprising first and second peptide chains, and optionally first and second peptide linkers; wherein the N-terminus of the IL-33 domain is connected to the C-terminus of the first peptide chain of the Fc domain directly or via the first peptide linker, and the N-terminus of the ST2 domain is connected to the C-terminus of the second peptide chain of the Fc domain directly or via the second peptide linker.

25. The fusion protein of claim 1 or 22, comprising first and second IL-33 domains, first and second ST2 domains, an Fc domain comprising first and second peptide chains, and optionally first, second, third, and fourth peptide linkers; wherein the N-terminus of the first IL-33 domain is connected to the C-terminus of the first ST2 domain directly or via the first peptide linker, and the N-terminus of the first ST2 domain is connected to the C-terminus of the first peptide chain of the Fc domain directly or via the second peptide linker; and wherein the N-terminus of the second IL-33 domain is connected to the C-terminus of the second ST2 domain directly or via the third peptide linker, and the N-terminus of the second ST2 domain is connected to the C-terminus of the second peptide chain of the Fc domain directly or via the fourth peptide linker.

26. The fusion protein of any one of claims 18 to 25, wherein the Fc domain is a hIgG1 Fc domain, a hIgG2 Fc domain, a hIgG4 Fc domain, or a mutein thereof.

27. The fusion protein of any one of claims 18 to 26, wherein the Fc domain is a hIgG1 Fc domain or a mutein thereof.

28. The fusion protein of any one of claims 18 to 26, wherein the Fc domain is a hIgG4 Fc domain or a mutein thereof.

29. The fusion protein of any one of claims 18 to 28, wherein the Fc domain comprises an amino acid sequence of SEQ ID NO: 55, 56, 57, 58, 59, 60, 61, 62, 63, 64, 65, or 66.

30. The fusion protein of any one of claims 18 to 29, wherein the Fc domain is in a knobs-in-holes configuration.

31. The fusion protein of claim 30, wherein the Fc domain comprises amino acid sequences of SEQ ID NO: 58 and 59, 60 and 61, 62 and 63, or 65 and 66 as a pair of peptide chains in a knobs-in-holes configuration.

32. The fusion protein of any one of claims 18 to 31, further comprising a programmed death-ligand 1 (PD-L1) binding domain.

33. The fusion protein of claim 32, wherein the PD-L1 binding domain is an anti-PD-L1 single domain antibody.

34. The fusion protein of claim 32 or 33, wherein the PD-L1 binding domain is an anti-PD-L1 V_{H}H single domain antibody.

35. The fusion protein of any one of claims 18 to 34, comprising an IL-33 domain, an ST2 domain, an anti-PD-L1 V_{H}H single domain antibody, and an Fc domain.

36. The fusion protein of any one of claims 18 to 35, comprising an IL-33 domain, an ST2 domain, first and second anti-PD-L1 V_{H}H single domain antibodies, an Fc domain comprising a first and second peptide chains, and optionally first and second linker; wherein the C-terminus of the first anti-PD-L1 V_{H}H single domain antibody is connected to the N-terminus of the first peptide chain of the Fc domain directly or via a peptide linker, the C-terminus of the first peptide chain of the Fc domain is connected to the N-terminus of the ST2 domain directly or via the first peptide linker, and the C-terminus of the ST2 domain is connected to the N-terminus of the IL-33 domain directly or via the second peptide linker; and wherein the C-terminus of the second anti-PD-L1 V_{H}H single domain antibody is connected to the N-terminus of the second peptide chain of the Fc domain directly or via a peptide linker.

37. The fusion protein of any one of claims 18 to 35, comprising an IL-33 domain, an ST2 domain, first and second anti-PD-L1 V_{H}H single domain antibodies, an Fc domain comprising a first and second peptide chains, and optionally first and second linker; wherein the C-terminus of the first anti-PD-L1 V_{H}H single domain is connected to the N-terminus of the first peptide chain of the Fc domain directly or via a peptide linker, and the C-terminus of the first peptide chain of the Fc domain is connected to the N-terminus of the IL-33 domain directly or via the first peptide linker; and wherein the C-terminus of the second anti-PD-L1 V_{H}H single domain antibody is connected to the N-terminus of the second peptide chain of the Fc domain directly or via a peptide linker, and the C-terminus of the second peptide chain of the Fc domain is connected to the N-terminus of the ST2 domain directly or via the second peptide linker.

38. The fusion protein of any one of claims 18 to 35, comprising first and second IL-33 domains, first and second ST2 domains, first and second anti-PD-L1 V_{H}H single domain antibodies, an Fc domain comprising a first and second peptide chains, and optionally first, second, third, and fourth peptide linkers; wherein the C-terminus of the first anti-PD-L1 V_{H}H single domain antibody is connected to the N-terminus of the first peptide chain of the Fc domain directly or via a peptide linker, the C-terminus of the first peptide chain of the Fc domain is connected to the N-terminus of the first ST2 domain directly or via the first peptide linker, and the C-terminus of the first ST2 domain is connected to the N-terminus of the first IL-33 domain directly or via the second peptide linker; and wherein the C-terminus of the second anti-PD-L1 V_{H}H single domain antibody is connected to the N-terminus of the second peptide chain of the Fc domain directly or via a peptide linker, the C-terminus of the second peptide chain of the Fc domain is connected to the N-terminus of the second ST2 domain directly or via the third peptide linker, and the C-terminus of the second ST2 domain is connected to the N-terminus of the second IL-33 domain directly or via the fourth peptide linker.

39. The fusion protein of any one of claims 32 to 38, wherein each anti-PD-L1 V_{H}H single domain antibody independently comprises a CDR1 of SEQ ID NO: 113, a CDR2 of SEQ ID NO: 114, and a CDR3 of SEQ ID NO: 115.

40. The fusion protein of any one of claims 32 to 39, wherein each anti-PD-L1 V_{H}H single domain antibody comprises the amino acid sequence of SEQ ID NO: 116.

41. The fusion protein of claim 32, wherein the anti-PD-L1 binding domain and the FC domain form parts of an intact anti-PD-L1 antibody comprising two light chains and two heavy chains.

42. The fusion protein of claim 32 or 40, comprising an IL-33 domain, an ST2 domain, and an intact anti-PD-L1 antibody comprising two light chains and two heavy chains.

43. The fusion protein of claim 41 or 42, comprising an IL-33 domain, an ST2 domain, an intact anti-PD-L1 antibody comprising first and second light chains and first and second heavy chains, and optionally first and second peptide linkers; wherein the N-terminus of the ST2 domain is connected to the C-terminus of the first heavy chain of the intact anti-PD-L1 antibody directly or via the first peptide linker; and the N-terminus of the IL-33 domain is connected to the C-terminus of the ST2 domain directly or via the second peptide linker.

44. The fusion protein of claim 41 or 42, comprising an IL-33 domain, an ST2 domain, an intact anti-PD-L1 antibody comprising first and second light chains and first and second heavy chains, and optionally first and second peptide linkers; wherein the N-terminus of the ST2 domain is connected to the C-terminus of the first heavy chain of the intact anti-PD-L1 antibody directly or via the first peptide linker; and the N-terminus of the IL-33 domain is connected to the C-terminus of the second heavy chain of the intact anti-PD-L1 antibody directly or via the second peptide linker.

45. The fusion protein of claim 41 or 42, comprising first and second IL-33 domains, first and second ST2 domains, an intact anti-PD-L1 antibody comprising first and second light chains and first and second heavy chains, and optionally first, second, third, and fourth peptide linkers; wherein the N-terminus of the first ST2 domain is connected to the C-terminus of the first heavy chain of the intact anti-PD-L1 antibody directly or via the first peptide linker, and the N-terminus of the first IL-33 domain is connected to the C-terminus of the first ST2 domain directly or via the second peptide linker; and wherein the N-terminus of the second ST2 domain is connected to the C-terminus of the second heavy chain of the intact anti-PD-L1 antibody directly or via the third peptide linker, and the N-terminus of the second IL-33 domain is connected to the C-terminus of the second ST2 domain directly or via the fourth peptide linker.

46. The fusion protein of any one of claims 41 to 45, wherein the intact PD-L1 antibody comprises:
(i) a CDRL1 of SEQ ID NO: 67, a CDRL2 of SEQ ID NO: 68, a CDRL3 of SEQ ID NO: 69, a CDRH1 of SEQ ID NO: 70, a CDRH2 of SEQ ID NO: 71, and a CDRH3 of SEQ ID NO: 72;
(ii) a CDRL1 of SEQ ID NO: 77, a CDRL2 of SEQ ID NO: 78, a CDRL3 of SEQ ID NO: 79, a CDRH1 of SEQ ID NO: 80, a CDRH2 of SEQ ID NO: 81, and a CDRH3 of SEQ ID NO: 82;
(iii) a CDRL1 of SEQ ID NO: 87, a CDRL2 of SEQ ID NO: 88, a CDRL3 of SEQ ID NO: 89, a CDRH1 of SEQ ID NO: 90, a CDRH2 of SEQ ID NO: 91, and a CDRH3 of SEQ ID NO: 92; or
(iv) a CDRL1 of SEQ ID NO: 97, a CDRL2 of SEQ ID NO: 98, a CDRL3 of SEQ ID NO: 99, a CDRH1 of SEQ ID NO: 100, a CDRH2 of SEQ ID NO: 101, and a CDRH3 of SEQ ID NO: 102.

47. The fusion protein of any one of claims 41 to 46, wherein the intact PD-L1 antibody comprises a CDRL1 of SEQ ID NO: 67, a CDRL2 of SEQ ID NO: 68, a CDRL3 of SEQ ID NO: 69, a CDRH1 of SEQ ID NO: 70, a CDRH2 of SEQ ID NO: 71, and a CDRH3 of SEQ ID NO: 72.

48. The fusion protein of any one of claims 41 to 47, wherein the intact PD-L1 antibody comprises a light chain variable region of SEQ ID NO: 73 and a heavy chain variable region of SEQ ID NO: 74.

49. The fusion protein of any one of claims 41 to 46, wherein the intact PD-L1 antibody comprises a CDRL1 of SEQ ID NO: 77, a CDRL2 of SEQ ID NO: 78, a CDRL3 of SEQ ID NO: 79, a CDRH1 of SEQ ID NO: 80, a CDRH2 of SEQ ID NO: 81, and a CDRH3 of SEQ ID NO: 82.

50. The fusion protein of any one of claims 41 to 46 and 49, wherein the intact PD-L1 antibody comprises a light chain variable region of SEQ ID NO: 83 and a heavy chain variable region of SEQ ID NO: 84.

51. The fusion protein of any one of claims 41 to 46, wherein the intact PD-L1 antibody comprises a CDRL1 of SEQ ID NO: 87, a CDRL2 of SEQ ID NO: 88, a CDRL3 of SEQ ID NO: 89, a CDRH1 of SEQ ID NO: 90, a CDRH2 of SEQ ID NO: 91, and a CDRH3 of SEQ ID NO: 92.

52. The fusion protein of any one of claims 41 to 46 and 51, wherein the intact PD-L1 antibody comprises a light chain variable region of SEQ ID NO: 93 and a heavy chain variable region of SEQ ID NO: 94.

53. The fusion protein of any one of claims 41 to 46, wherein the intact PD-L1 antibody comprises a CDRL1 of SEQ ID NO: 97, a CDRL2 of SEQ ID NO: 98, a CDRL3 of SEQ ID NO: 99, a CDRH1 of SEQ ID NO: 100, a CDRH2 of SEQ ID NO: 101, and a CDRH3 of SEQ ID NO: 102.

54. The fusion protein of any one of claims 41 to 46 and 53, wherein the intact PD-L1 antibody comprises a light chain variable region of SEQ ID NO: 103 and a heavy chain variable region of SEQ ID NO: 104.

55. The fusion protein of any one of claims 41 to 54, wherein the intact PD-L1 antibody comprises: a light chain of SEQ ID NO: 75 and a light chain of SEQ ID NO: 76; a light chain of SEQ ID NO: 85 and a light chain of SEQ ID NO: 86; a light chain of SEQ ID NO: 95 and a light chain of SEQ ID NO: 96; a light chain of SEQ ID NO: 105 and a light chain of SEQ ID NO: 106; a light chain of SEQ ID NO: 107 and a light chain of SEQ ID NO: 108; a light chain of SEQ ID NO: 109 and a light chain of SEQ ID NO: 110; or a light chain of SEQ ID NO: 111 and a light chain of SEQ ID NO: 112.

56. The fusion protein of any one of claims 18 to 31, further comprising a mesothelin (MSLN) binding domain.

57. The fusion protein of claim 56, wherein the MSLN binding domain is an anti-MSLN single domain antibody.

58. The fusion protein of claim 56 or 57, wherein the MSLN binding domain is an anti-MSLN V_{H}H single domain antibody.

59. The fusion protein of any one of claims 56 to 58, comprising an IL-33 domain, an ST2 domain, an anti-MSLN V_{H}H single domain antibody, and an Fc domain.

60. The fusion protein of any one of claims 56 to 59, comprising an IL-33 domain, an ST2 domain, first and second anti-MSLN V_{H}H single domain antibodies, an Fc domain comprising a first and second peptide chains, and optionally first and second peptide linkers; wherein the C-terminus of the first anti-MSLN V_{H}H single domain antibody is connected to the N-terminus of the first peptide chain of the Fc domain directly or via a peptide linker, the C-terminus of the first peptide chain of the Fc domain is connected to the N-terminus of the ST2 domain directly or via the first peptide linker, and the C-terminus of the ST2 domain is connected to the N-terminus of the IL-33 domain directly or via the second peptide linker; and wherein the C-terminus of the second anti-MSLN V_{H}H single domain antibody is connected to the N-terminus of the second peptide chain of the Fc domain directly or via a peptide linker.

61. The fusion protein of any one of claims 56 to 59, comprising an IL-33 domain, an ST2 domain, first and second anti-MSLN V_{H}H single domain antibodies, an Fc domain comprising a first and second peptide chains, and optionally first and second peptide linkers; wherein the C-terminus of the first anti-MSLN V_{H}H single domain is connected to the N-terminus of the first peptide chain of the Fc domain directly or via a peptide linker, and the C-terminus of the first peptide chain of the Fc domain is connected to the N-terminus of the IL-33 domain directly or via the first peptide linker; and wherein the C-terminus of the second anti-MSLN V_{H}H single domain antibody is connected to the N-terminus of the second peptide chain of the Fc domain directly or via a peptide linker, and the C-terminus of the second peptide chain of the Fc domain is connected to the N-terminus of the ST2 domain directly or via the second peptide linker.

62. The fusion protein of any one of claims 56 to 59, comprising first and second IL-33 domains, first and second ST2 domains, first and second anti-MSLN V_{H}H single domain antibodies, an Fc domain comprising a first and second peptide chains, and optionally first, second, third, and fourth peptide linkers; wherein the C-terminus of the first anti-MSLN V_{H}H single domain antibody is connected to the N-terminus of the first peptide chain of the Fc domain directly or via a peptide linker, the C-terminus of the first peptide chain of the Fc domain is connected to the N-terminus of the first ST2 domain directly or via the first peptide linker, and the C-terminus of the first ST2 domain is connected to the N-terminus of the first IL-33 domain directly or via the second peptide linker; and wherein the C-terminus of the second anti-MSLN V_{H}H single domain antibody is connected to the N-terminus of the second peptide chain of the Fc domain directly or via a peptide linker, the C-terminus of the second peptide chain of the Fc domain is connected to the N-terminus of the second ST2 domain directly or via the third peptide linker, and the C-terminus of the second ST2 domain is connected to the N-terminus of the second IL-33 domain directly or via the fourth peptide linker.

63. The fusion protein of any one of claims 56 to 62, wherein each anti-MSLN V_{H}H single domain antibody comprises a CDR1 of SEQ ID NO: 117, a CDR2 of SEQ ID NO: 118, and a CDR3 of SEQ ID NO: 119.

64. The fusion protein of any one of claims 56 to 63, wherein each anti-MSLN single domain antibody comprises the amino acid sequence of SEQ ID NO: 124.

65. The fusion protein of any one of claims 1 to 64, wherein the IL-33 domain comprises an amino acid sequence of SEQ ID NO: 125, 126, 127, 128, or 129; or a mutein or variant thereof.

66. The fusion protein of any one of claims 1 to 65, wherein the IL-33 domain comprises an amino acid sequence of SEQ ID NO: 125, or a mutein or variant thereof.

67. The fusion protein of any one of claims 1 to 65, wherein the IL-33 domain comprises an amino acid sequence of SEQ ID NO: 126, or a mutein or variant thereof.

68. The fusion protein of any one of claims 1 to 65, wherein the IL-33 domain comprises an amino acid sequence of SEQ ID NO: 127, or a mutein or variant thereof.

69. The fusion protein of any one of claims 1 to 65, wherein the IL-33 domain comprises an amino acid sequence of SEQ ID NO: 128, or a mutein or variant thereof.

70. The fusion protein of any one of claims 1 to 65, wherein the IL-33 domain comprises an amino acid sequence of SEQ ID NO: 129, or a mutein or variant thereof.

71. The fusion protein of any one of claims 1 to 70, wherein the ST2 domain comprises an amino acid sequence of SEQ ID NO: 130 or 131, or a mutein or variant thereof.

72. The fusion protein of any one of claims 1 to 71, wherein the ST2 domain comprises an amino acid sequence of SEQ ID NO: 130, or a mutein or variant thereof.

73. The fusion protein of any one of claims 1 to 71, wherein the ST2 domain comprises an amino acid sequence of SEQ ID NO: 131, or a mutein or variant thereof.

74. The fusion protein of any one of claims 1 to 73, wherein each peptide linker independently comprises an amino acid sequence selected from GSG and SEQ ID NOs: 16 to 54.

75. The fusion protein of any one of claims 1 to 74, wherein each peptide linker independently comprises an amino acid sequence of SEQ ID NO: 22, 25, or 54.

76. The fusion protein of claim 1, wherein the fusion protein is:
fusion protein A1: comprising an amino acid sequence of SEQ ID NO: 132;
fusion protein A2: comprising an amino acid sequence of SEQ ID NO: 133;
fusion protein A3: comprising an amino acid sequence of SEQ ID NO: 134;
fusion protein A4: comprising an amino acid sequence of SEQ ID NO: 135;
fusion protein A5: comprising an amino acid sequence of SEQ ID NO: 136;
fusion protein A6: comprising an amino acid sequence of SEQ ID NO: 137;
fusion protein A7: comprising an amino acid sequence of SEQ ID NO: 138;
fusion protein A8: comprising an amino acid sequence of SEQ ID NO: 139;
fusion protein A9: comprising an amino acid sequence of SEQ ID NO: 140;
fusion protein A10: comprising amino acid sequences of SEQ ID NOs: 141 and 143;
fusion protein A11: comprising amino acid sequences of SEQ ID NOs: 142 and 143;
fusion protein A12: comprising amino acid sequences of SEQ ID NOs: 144 and 145;
fusion protein A13: comprising amino acid sequences of SEQ ID NOs: 146 and 148;
fusion protein A14: comprising amino acid sequences of SEQ ID NOs: 147 and 148;
fusion protein A15: comprising an amino acid sequence of SEQ ID NO: 149;
fusion protein A16: comprising an amino acid sequence of SEQ ID NO: 150;
fusion protein A17: comprising amino acid sequences of SEQ ID NOs: 151, 153, and 159;
fusion protein A18: comprising amino acid sequences of SEQ ID NOs: 152, 153, and 159;
fusion protein A19: comprising amino acid sequences of SEQ ID NOs: 154, 156, and
fusion protein A20: comprising amino acid sequences of SEQ ID NOs: 155, 156, and 159;
fusion protein A21: comprising amino acid sequences of SEQ ID NOs: 157 and 159;
fusion protein A22: comprising amino acid sequences of SEQ ID NOs: 158 and 159;
fusion protein A23: comprising amino acid sequences of SEQ ID NOs: 160 and 162;
fusion protein A24: comprising amino acid sequences of SEQ ID NOs: 161 and 162;
fusion protein A25: comprising amino acid sequences of SEQ ID NOs: 163 and 164;
fusion protein A26: comprising amino acid sequences of SEQ ID NOs: 165 and 166; or
fusion protein A27: comprising an amino acid sequence of SEQ ID NO: 167.

77. A fusion protein complex comprising a fusion protein of any one of claims 1 to 76, and a free IL-33 or ST2; wherein an IL-33 domain of the fusion protein is complexed with a free ST2, or an ST2 domain of the fusion protein is complexed with a free IL-33.

78. The fusion protein complex of claim 77, comprising an IL-33 fusion protein and an ST2; wherein the IL-33 fusion protein comprises an IL-33 domain and an anti-HSA V_{H}H single domain antibody; wherein the C-terminus of the anti-HSA V_{H}H single domain antibody is connected to the N-terminus of the IL-33 domain directly or via a peptide linker; and wherein each IL-33 domain in the IL-33 fusion protein is complexed with one ST2.

79. The fusion protein complex of claim 77, comprising an IL-33 fusion protein and two ST2; wherein the IL-33 fusion protein comprises first and second IL-33 domains and an Fc domain comprising first and second peptide chains; wherein the C-terminus of the first peptide chain of the Fc domain is connected to the N-terminus of the first IL-33 domain directly or via a peptide linker, and the C-terminus of the second peptide chain of the Fc domain is connected to the N-terminus of the second IL-33 domain directly or via a peptide linker; and wherein each IL-33 domain in the IL-33 fusion protein is complexed with one ST2.

80. The fusion protein complex of claim 77, comprising an ST2 fusion protein and an IL-33; wherein the ST2 fusion protein comprises an ST2 domain and an anti-HSA V_{H}H single domain antibody; wherein the C-terminus of the ST2 domain is connected to the N-terminus of the anti-HSA V_{H}H single domain antibody directly or via a peptide linker; and wherein each ST2 domain in the ST2 fusion protein is complexed with one IL-33.

81. The fusion protein complex of claim 77, comprising an ST2 fusion protein and two IL-33; wherein the ST2 fusion protein comprises first and second ST2 domains and an Fc domain comprising first and second peptide chains; wherein the C-terminus of the first ST2 domain is connected to the N-terminus of the first peptide chain of the Fc domain directly or via a peptide linker; and the C-terminus of the second ST2 domain is connected to the N-terminus of the second peptide chain of the Fc domain directly or via a peptide linker; and wherein each ST2 domain in the ST2 fusion protein is complexed with one IL-33.

82. The fusion protein complex of claim 77, comprising an ST2 fusion protein and two IL-33; wherein the ST2 fusion protein comprises first and second ST2 domains and an Fc domain comprising first and second peptide chains; wherein the C-terminus of the first peptide chain of the Fc domain is connected to the N-terminus of the first ST2 domain directly or via a peptide linker; and the C-terminus of the second peptide chain of the Fc domain is connected to the N-terminus of the second ST2 domain directly or via a peptide linker; and wherein each ST2 domain in the ST2 fusion protein is complexed with one IL-33.

83. The fusion protein complex of claim 77, wherein the fusion protein complex is:
fusion protein complex C1: comprising (i) a fusion protein comprising an amino acid sequence of SEQ ID NO: 132 and (ii) a free ST2 of SEQ ID NO: 130;
fusion protein complex C2: comprising (i) a fusion protein comprising an amino acid sequence of SEQ ID NO: 132 and (ii) a free ST2 of SEQ ID NO: 131;
fusion protein complex C3: comprising (i) a fusion protein comprising an amino acid sequence of SEQ ID NO: 133 and (ii) a free ST2 of SEQ ID NO: 130;
fusion protein complex C4: comprising (i) a fusion protein comprising an amino acid sequence of SEQ ID NO: 133 and (ii) a free ST2 of SEQ ID NO: 131;
fusion protein complex C5: comprising an amino acid sequence of SEQ ID NO: 138; and (ii) a free ST2 of SEQ ID NO: 130;
fusion protein complex C6: comprising an amino acid sequence of SEQ ID NO: 138; and (ii) a free ST2 of SEQ ID NO: 131;
fusion protein complex C7: comprising an amino acid sequence of SEQ ID NO: 139; and (ii) a free ST2 of SEQ ID NO: 130;
fusion protein complex C8: comprising an amino acid sequence of SEQ ID NO: 139; and (ii) a free ST2 of SEQ ID NO: 131;
fusion protein complex C9: comprising an amino acid sequence of SEQ ID NO: 140; and (ii) a free IL-33 of SEQ ID NO: 126;
fusion protein complex C10: comprising an amino acid sequence of SEQ ID NO: 140; and (ii) a free IL-33 mutein of SEQ ID NO: 127;
fusion protein complex C11: comprising an amino acid sequence of SEQ ID NO: 140; and (ii) a free IL-33 of SEQ ID NO: 128; or
fusion protein complex C12: comprising an amino acid sequence of SEQ ID NO: 140; and (ii) a free IL-33 of SEQ ID NO: 129.

84. A pharmaceutical composition comprising a fusion protein of any one of claims 1 to 76 or a fusion protein complex of any one of claims 77 to 83, and a pharmaceutically acceptable excipient.

85. The pharmaceutical composition of claim 84, wherein the pharmaceutical composition is in single dosage form.

86. The pharmaceutical composition of claim 84 or 85, wherein the pharmaceutical composition is a solid.

87. The pharmaceutical composition of claim 84 or 85, wherein the pharmaceutical composition is in a parenteral dosage form.

88. The pharmaceutical composition of claim 87, the pharmaceutical composition is in an intravenous dosage form.

89. The pharmaceutical composition of any one of claims 84, 85, 87, and 88, wherein the pharmaceutical composition is a solution.

90. A method of treating one or more symptoms of treating, preventing, or ameliorating one or more symptoms of an IL-33-mediated disorder, disease, or condition in a subject, comprising administering to the subject in need thereof a therapeutically effective amount of a fusion protein of any one of claims 1 to 76, a fusion protein complex of any one of claims 77 to 83, or a pharmaceutical composition of any one of claims 84 to 89.

91. The method of claim 90, wherein the IL-33-mediated disorder, disease, or condition is a proliferative disease.

92. The method of claim 91, wherein the proliferative disease is cancer.

93. The method of claim 92, wherein the cancer is melanoma.

94. The method of claim 92 or 93, wherein the cancer is relapsed or refractory.

95. The method of any one of claims 92 to 94, wherein the cancer is metastatic.

96. The method of any one of claims 92 to 95, wherein the cancer is drug-resistant.

97. The method of any one of claims 90 to 96, wherein the subject is a human.

98. A method of inhibiting the growth of a cell, comprising contacting the cell with an effective amount of a fusion protein of any one of claims 1 to 76, a fusion protein complex of any one of claims 77 to 83, or a pharmaceutical composition of any one of claims 84 to 89.

99. The method of claim 98, wherein the cell is a cancerous cell.
